(19) United States
(12) Patent Application Publication
(10) Pub. No.: US 2025/0376728 A1
(43) Pub. Date: Dec. 11, 2025

(54) PLATFORM FOR MONITORING TRANSPLANT REJECTION

(71) Applicant: The Regents of the University of Michigan, Ann Arbor, MI (US)

(72) Inventors: Russell Urie, Ann Arbor, MI (US); Aaron Morris, Ann Arbor, MI (US); Daniel Goldstein, Ann Arbor, MI (US); Lonnie Shea, Ann Arbor, MI (US)

(21) Appl. No.: 18/837,653

(22) PCT Filed: Feb. 14, 2023
(86) PCT No.: PCT/US23/12997

§ 371 Date: Mar. 6, 2025

Related U.S. Application Data
(60) Provisional application No. 63/310,201, filed on Feb. 15, 2022.

(51) Int. Cl. C12Q 1/6883; C12Q 1/6809

(52) U.S. Cl. CPC C12Q 1/6883; C12Q 1/6809; C12Q 2600/106

(57) ABSTRACT

The present disclosure relates to materials and methods for evaluation of transplant rejection in a subject. In particular. provided herein are synthetic scaffolds and methods of use thereof for early diagnosis of transplant rejection in a subject.

### STATEMENT REGARDING RELATED APPLICATIONS

This application claims priority to U.S. Provisional Patent Application No. 63/310,201, filed Feb. 15, 2022, the entire contents of which are incorporated herein by reference for all purposes.

### FIELD

The present disclosure relates to materials and methods for evaluation of transplant rejection in a subject. In particular, provided herein are synthetic scaffolds and methods of use thereof for early diagnosis of transplant rejection in a subject.

### BACKGROUND

Over 36,000 solid organ transplants are conducted annually in the US. Immunosuppressive drugs protect these donor grafts from acute rejection but increase the risk of opportunistic infections and cancer, especially in pediatric transplant recipients, who require immune suppression for decades. As there is no method for determining which grafts will be rejected, immunosuppression is aggressively applied in a one-size-fits-all approach. Furthermore, even with immunosuppression, rejection events can occur. If caught early, interventions may be applied to minimize harm to the subject and the transplanted organ. Accordingly, what is needed are methods for evaluating risk of transplant rejection prior to rejection onset that allow for personalized immunosuppression regimes.

### SUMMARY

In some aspects, provided herein are methods involving evaluating a microenvironment of a synthetic scaffold implanted in a subject that has received a transplant. The methods provided herein find use in evaluating transplant rejection status in the subject. For example, the methods provided herein can be used to determine whether a subject that has received a transplant is experiencing rejection or at risk of experiencing rejection, before clinical rejection symptoms are present in the subject. Accordingly, the synthetic scaffolds and methods of use thereof described herein provide an innovative strategy for evaluating transplant rejection in a subject that negates the need for an invasive biopsy, and that provides sufficiently early (e.g. pre-symptomatic) detection of rejection in the subject and subsequent determination of what treatment steps should be taken in order to potentially save the graft.

In some embodiments, provided herein is a method comprising obtaining at least one sample from a microenvironment of a synthetic scaffold implanted in a subject that has received a transplant, and measuring an expression level of at least one RNA, at least one gene, at least one cell type, and/or at least one protein in the at least one sample. In some embodiments the transplant is an allogeneic transplant. In some embodiments, the transplant is a xenogeneic transplant. In some embodiments, the transplant is a heart transplant. In some embodiments, the transplant is a skin transplant. In some embodiments, the subject has received at least one anti-rejection therapy. In some embodiments, the least one anti-rejection therapy is selected from an immunosuppressive agent and an antibody.

In some embodiments, the at least one sample comprises at least 50% dendritic cells relative to the total amount of CD45+ cells in the sample. In some embodiments, the at least one sample comprises at least 60% dendritic cells relative to the total amount of CD45+ cells in the sample.

In some embodiments, the at least one sample is obtained no more than 7 days after the subject has received the transplant. For example, in some embodiments the at least one sample is obtained 7 days, 6 days, 5 days, 4 days, 3 days, 2 days, 24 hours, 12 hours, or less than 12 hours after the subject has received the transplant. In some embodiments, the at least one sample is obtained no more than 5 days after the subject has received the transplant. For example, in some embodiments the at least one sample is obtained 5 days, 4 days, 3 days, 2 days, 24 hours, 12 hours, or less than 12 hours after the subject has received the transplant.

In some embodiments, the methods provided herein involve obtaining more than one sample from the microenvironment of the synthetic scaffold. In some embodiments, the at least one sample comprises a first sample obtained at a first time point after the subject has received the transplant and a second sample obtained at a second time point after the subject has received the transplant. In some embodiments, the first sample is obtained within 7 days after the subject has received the transplant, and the second sample is obtained at least 24 hours after the first sample. In some embodiments, the first sample is obtained within 7 days after the subject has received the transplant, and the second sample is obtained 1-14 days after the first sample. In some embodiments, the first sample is obtained within 7 days after the subject has received the transplant, and the second sample is obtained 1-7 days after the first sample. In some embodiments, the first sample is obtained within 5 days after the subject has received the transplant, and the second sample is obtained at least 24 hours after the first sample. In some embodiments, the first sample is obtained within 5 days after the subject has received the transplant, and the second sample is obtained 1-14 days after the first sample. In some embodiments, the first sample is obtained within 5 days after the subject has received the transplant, and the second sample is obtained 1-7 days after the first sample.

In some embodiments, the method comprises measuring an expression level of a panel of genes in the at least one sample. In some embodiments, the panel of genes comprises at least 3 genes. In some embodiments, the panel of genes comprises 3-50 genes. In some embodiments, the panel of genes comprises 6-25 genes.

In some aspects, provided herein are methods of monitoring transplant rejection in a subject. In some embodiments, methods of monitoring transplant rejection comprises obtaining at least one sample from a microenvironment of a synthetic scaffold implanted in a subject that has received a transplant, measuring an expression level or amount of at least one RNA, at least one gene, at least one cell type, and/or at least one protein in the at least one sample, and determining transplant rejection status in the subject based upon the expression level or amount of the at least one RNA, at least one gene, at least one cell type, and/or at least one protein in the at least one sample. In some embodiments, the at least one sample is obtained no more than 7days after the subject has received the transplant. For example, in some embodiments the at least one sample is obtained 7 days, 6 days, 5 days, 4 days, 3 days, 2 days, 24 hours, 12 hours, or less than 12 hours after the subject has received the transplant. In some embodiments, the at least one sample is obtained no more than 5 days after the subject has received the transplant. For example, in some embodiments the at least one sample is obtained 5 days, 4 days, 3 days, 2 days, 24 hours, 12 hours, or less than 12 hours after the subject has received the transplant.

In some embodiments, the at least one sample comprises at least 50% dendritic cells relative to the total amount of CD45+ cells in the sample. In some embodiments, the at least one sample comprises at least 60% dendritic cells relative to the total amount of CD45+ cells in the sample.

In some embodiments, determining transplant rejection status in the subject comprises determining that the patient is at risk of or currently experiencing transplant rejection when the expression level or amount of the at least one RNA, at least one gene, at least one cell type, and/or at least one protein in the sample is increased or decreased compared to a reference level for the at least one RNA, at least one gene, at least one cell type, and/or at least one protein. In some embodiments, determining transplant rejection status comprises measuring an expression level of a panel of genes in the at least one sample. In some embodiments, the panel of genes comprises at least 3 genes. In some embodiments, the panel of genes comprises 3-50 genes. In some embodiments, the panel of genes comprises 6-25 genes. In some embodiments, the method comprises determining that the patient is at risk of or currently experiencing transplant rejection when the expression level or amount of one or more genes in the panel of genes is increased or decreased compared to a reference level. In some embodiments, the method further comprises providing an anti-rejection therapy to the subject determined to be at risk of or currently experiencing transplant rejection.

In some embodiments, the method comprises obtaining more than one sample from the microenvironment of the synthetic scaffold. In some embodiments, the at least one sample comprises a first sample obtained at a first time point after the subject has received the transplant and a second sample obtained at a second time point after the subject has received the transplant. In some embodiments, the first sample is obtained within 7 days after the subject has received the transplant, and the second sample is obtained at least 24 hours after the first sample. In some embodiments, the first sample is obtained within 7 days after the subject has received the transplant, and the second sample is obtained 1-14 days after the first sample. In some embodiments, the first sample is obtained within 7 days after the subject has received the transplant, and the second sample is obtained 1-7 days after the first sample. In some embodiments, the first sample is obtained within 5 days after the subject has received the transplant, and the second sample is obtained at least 24 hours after the first sample. In some embodiments, the first sample is obtained within 5 days after the subject has received the transplant, and the second sample is obtained 1-14 days after the first sample. In some embodiments, the first sample is obtained within 5 days after the subject has received the transplant, and the second sample is obtained 1-7 days after the first sample.

In some embodiments, determining transplant rejection status in the subject comprises determining that the patient is at risk of or currently experiencing transplant rejection when the expression level or amount of the at least one RNA, at least one gene, at least one cell type, and/or at least one protein in the second sample is increased or decreased compared to the expression level or amount in the first sample. In some embodiments, determining transplant rejection status comprises measuring an expression level of a panel of genes in the first sample and in the second sample. In some embodiments, the panel of genes comprises at least 3 genes.

In some embodiments, the panel of genes comprises 3-50 genes. In some embodiments, the panel of genes comprises 6-25 genes. In some embodiments, the method comprises determining that the patient is at risk of or currently experiencing transplant rejection when the expression level or amount of one or more genes in the panel of genes is increased or decreased in the second sample compared to the first sample. In some embodiments, the method further comprises providing an anti-rejection therapy to the subject determined to be at risk of or currently experiencing transplant rejection.

In some embodiments the transplant is an allogeneic transplant. In some embodiments, the transplant is a xenogeneic transplant. In some embodiments, the transplant is a heart transplant. In some embodiments, the transplant is a skin transplant. In some embodiments, the subject has received at least one anti-rejection therapy. In some embodiments, the least one anti-rejection therapy is selected from an immunosuppressive agent and an antibody.

### DESCRIPTION OF THE DRAWINGS

FIG. 1 is a schematic showing an exemplary protocol for a heart transplant and subsequent monitoring using the materials and methods described herein. In this embodiment, heart transplant occurs at day −24. Scaffolds are implanted at day −14. Adoptive transfer of T cells starts from at day 0. Scaffolds are biopsied at Days 0, 5, 9, and 15 for analysis by RNAseq and histology. Source of T cells is from the spleen of sex and age matched C57B16 mice.

FIGS. 2A-2B show measurements of transplanted heart function. FIG. 2A is an image at day 0 and day 15 following transplant. FIG. 2B shows heart transplant size (top graph) and score based upon strength of heartbeat and swelling (bottom graph).

FIGS. 3A-3B show a comparison of syngeneic and allogeneic heart transplants. FIG. 3A shows histology (H&E staining) from a syngeneic heart graft at day 15 post adoptive T cell transfer. The transplant received a rejection score of 0, as there was no swelling observed and a strong heartbeat. FIG. 3B shows a histology (H&E staining) from an allogeneic heart graft at day 15 post adoptive T cell transfer. The transplant received a rejection score of 3, as it was very swollen and there was no heartbeat. Images are representative from n=3 animals per group, with all microscope settings identical.

FIGS. 4A-4B show a comparison of scaffolds obtained from animals with syngeneic and allogeneic heart transplants. FIG. 4A shows histology (H&E staining) from a scaffold from a syngeneic heart graft at day 9. The transplant received a rejection score of 0, as there was no swelling observed and a strong heartbeat. FIG. 4B shows a histology (H&E staining) from a scaffold from an allogeneic heart graft at day 9. The transplant received a rejection score of 3, as it was very swollen and there was no heartbeat. Images are representative from n=3 animals per group, with all microscope settings identical.

FIGS. 5A-5B show a comparison of scaffolds obtained from animals with syngeneic and allogeneic heart transplants. FIG. 5A shows histology (H&E staining) from a scaffold from a syngeneic heart graft at day 15. The transplant received a rejection score of 0, as there was no swelling observed and a strong heartbeat. FIG. 5B shows a histology (H&E staining) from a scaffold from an allogeneic heart graft at day 15. The transplant received a rejection score of 3, as it was very swollen and there was no heartbeat. Images are representative from n=3 animals per group, with all microscope settings identical.

FIG. 6 is a schematic showing an exemplary protocol for using the materials disclosed herein for monitoring skin graft rejection. From day −28 to day 0 skin transplants are performed and scaffolds are implanted. At day 0, T cells are transferred. Scaffolds are biopsied at day 0, day 7, and day 13. T cells are from the spleen of sex and age matched C57B16 mice.

FIG. 7 shows images of the skin graft site in training and validation cohorts at various time points. Images are representative images of n=3 animals per group.

FIG. 8 shows analysis of RNAseq data to obtain a skin rejection scaffold signature of rejection associated transcripts (RATs). RNAseq data was analyzed for the multiple samples. Raw counts were normalized by DESeq2. Each gene underwent hypothesis testing comparing rejecting transplants to healthy transplants to identify a subset of differentially expressed genes. This subset of genes was then analyzed for greatest positive or negative fold change and false discovery rate to determine a panel of fewer than 30 genes that distinguish between scaffolds from mice with rejecting and non-rejecting transplants. A volcano plot of 14 genes identified is shown in FIG. 8A. A heatmap of z-scores for the 14 genes is shown in FIG. 8B.

FIGS. 9A-9B show a two metric scoring system based upon differential gene expression. FIG. 9A shows singular value decomposition SVD (supervised) and Random Forest (unsupervised). The gene expression can then be converted to two numbers that reflect a position in the graph of FIG. 9B. Scaffolds from healthy or non-rejecting mice are in blue, and generally had a low score by both SVD and random forest. Scaffolds from early or late rejecting transplanted mice had a high score in both scoring systems.

FIGS. 10A-10B demonstrate that scaffolds do not affect rejection following a skin graft. FIG. 10A shows images from allogeneic skin grafts with and without scaffolds, and from syngeneic skin grafts with scaffolds. FIG. 10B quantifies rejection score for all groups. Syngeneic graft do not reject, regardless of the presence of scaffolds.

FIGS. 11A-11I show the synthetic immunological niche during skin graft rejection. FIG. 11A is a schematic representation of immunological niche scaffolds as a bridge between the local immune responses at the allograft and the systemic immune responses present in the blood and hematopoietic tissues. FIG. 11B is a schematic representation of scaffold implants applied to monitor acute cellular alloreactivity of murine skin transplants in an adoptive transfer acute cellular allograft rejection model. An exemplary timeline is shown. FIG. 11C shows representative images of skin grafts on dorsum of C57B1/6 recipients. Top row: BALB/c tail skin grafts on C57BL/6 RAG2^{-/-} recipients (allogeneic). Arrow indicates early symptoms of rejection at graft on day 9 following adoptive T cell transfer. Bottom row: C57BL/6 tail skin grafts on C57BL/6 RAG2^{-/-} recipients (syngeneic). FIG. 11D shows skin grafts scored following adoptive T cell transfer by severity of rejection, n=6 graft recipients. Plot of allograft recipients with scaffold implants colored according to stage of allograft rejection. FIG. 11E shows cross-sectional images of 5 mm diameter scaffold explant, stained by hematoxylin and eosin. Images representative of n=3 scaffolds from independent mice. FIG. 11F shows insert at 20× magnification, scale bar=250 μm. FIG. 11G shows CD45+ cells as a fraction of all live cells found in the scaffold implant (left), the skin graft (center), and the blood (right) at various timepoints after adoptive T cell transfer, n=4 graft recipients. Plot of allograft recipients colored according to stage of allograft rejection. FIGS. 11H-11J show proportions of immune cell types in allogeneic and syngeneic skin transplant recipients in the (FIG. 11H) scaffold implants, (FIG. 111) the skin graft, and (FIG. 11J) the blood, n=4 graft recipients.

FIGS. 12A-12F show gene expression at the synthetic immunological niche during acute cellular skin allograft rejection. FIG. 12A is a representation of an exemplary gene expression sequencing analysis method. FIG. 12B shows most highly enriched immune pathways of presymptomatic ACAR (day 7), compared to late ACAR (day 13) at the scaffold. FIG. 12C shows clustered heatmap of an 18-gene panel. FIG. 12D shows principal component clustering of scaffolds based on the 18-gene panel. FIG. 12E shows clustered heatmap of a 6-gene panel. FIG. 12F shows principal component clustering of scaffolds based on the 6-gene panel. FIG. 12E, FIG. 12G show individual scaffold samples. All ellipses=70% CI.

FIGS. 13A-13H show gene expression at the synthetic immunological niche during acute cellular heart allograft rejection. FIG. 13A is a schematic representation of scaffold implants applied to monitor acute cellular alloreactivity of murine heart transplants in an adoptive transfer acute cellular allograft rejection model. FIG. 13B shows heart grafts scored following adoptive T cell transfer by severity of rejection, n=10 graft recipients. Plot of allograft recipients with scaffold implants colored according to stage of allograft rejection. (FIG. 13C,FIG. 13D show cross-sectional images of (FIG. 13C) syngeneic and (FIG. 13D) allogeneic heart grafts 14 days after adoptive T cell transfer, stained by hematoxylin and eosin. Images representative of n=3 heart grafts from independent graft recipients. Scale bar=250 μm. FIG. 13E shows a clustered heatmap of Elastic Net derived 17-gene panel for distinguishing ACAR. (FIG. 13F, FIG. 13G shows unsupervised SVD and supervised RF scoring of samples based on (FIG. 13F) 17-gene panel derived at the scaffold and (FIG. 13G) Allomap test murine orthologs at the scaffold. All ellipses=70% CI. FIG. 13H shows kidney biopsy canonical markers of graft rejection expressed at the scaffold during stages of HTx ACAR. * p<0.05, ** p<0.01, #pooled ACAR samples p<0.05, ##pooled ACAR samples p<0.01.

FIGS. 14A-14E show that scaffolds identify conserved biomarkers of ACAR across allograft types, skin grafts and heart grafts. FIG. 14A shows 25 pathways identified by Elastic Net regression of GSVA converted gene expression which sparsely distinguish between stages of ACAR. FIG. 14B shows principal component clustering of scaffolds based on the 25-pathway set. FIG. 14C shows logz fold change of gene expression at the scaffold comparing ACAR to healthy recipients in STx and HTx where genes with a false discovery rate (FDR) above 0.1 were excluded. FIG. 14D shows a clustered heatmap of Elastic Net derived 13-gene panel for distinguishing ACAR in both transplant types. FIG. 14E shows principal component clustering of scaffolds based on a 13-gene panel.

FIGS. 15A-15E show early and specific detection of alloreactivity in skin graft rejection via the scaffold. FIG. 15A shows graft survival of RAG2″-STx recipients following high or low T cell adoptive transfer to initiate ACAR. Non-survival events defined as the first day at which graft wounding is present. n=6. FIG. 15B shows weight loss as a measure of infection severity for mice inoculated with influenza virus. n=5. FIG. 15C shows principal component clustering of scaffolds based on a previously derived 13-gene panel for distinguishing pre-symptomatic ACAR from influenza infection. n=3 or 4. FIG. 15D shows graft survival of C57B1/6 STx recipients following high or low T cell depleting antibodies to delay ACAR. Non-survival events are defined as the first day at which graft wounding is present. n=7. FIG. 15E shows receiver-operator characteristic curve for sensitivity and specificity of identifying pre-symptomatic ACAR using the scaffold-derived 13-gene panel. n=3 or 4.

FIG. 16 shows longitudinal STx images for scoring grafts.

FIG. 17 shows immune cell ratios during various stages of ACAR in STx at the scaffold implant (top row), skin graft middle row), and in the blood (bottom row).

FIGS. 18A, 18B, 18C, 18D, 18E, 18F, and 18H show gene expression networks of the top differentially-enriched pathways in STx ACAR.

FIGS. 19A, 19B, and 19C show elastic net-derived GSVA scored pathways which distinguish ACAR from healthy STx recipients.

FIGS. 20A, 20B, and 20C show significant genes according to non-parametric t-test scores between healthy STx recipients and ACAR, with the principal component loadings of each gene in the sparse 18-gene panel.

FIGS. 21A-21B show histology of the HTx graft before adoptive T cell transfer, and swelling of the allografts as ACAR progresses.

FIGS. 22A, 22B, 22C, and 2l D show Principal component loadings and analysis of HTx ACAR panel.

FIGS. 23A-23B show principal component loadings and analysis of combined ACAR pathways.

FIGS. 24A, 24B, 24C, and 24D shows differential pathway enrichment between stages of ACAR.

FIGS. 25A-25B show principal component loadings and analysis of combined ACAR panel.

FIGS. 26A, 26B, and 26C show selection of housekeeping genes from combined ACAR for PCR controls.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “rejection” or “transplant rejection” is used herein in the broadest sense and refers to any stage or severity of rejection in the subject. Various immunologic mechanisms of rejection may be experienced by the subject depending on the source of the transplant, the duration of time that has passed since transplant, the type of anti-rejection therapy provided to the subject, and the like. In some embodiments, the term “rejection” refers to acute rejection. The term “acute rejection” refers to a stage of rejection that develops with the formation of cellular immunity. Acute rejection may occur as early as one week after transplant with the highest risk being in the first three months. It is believed that acute rejection is mediated by mononuclear macrophages and T-lymphocytes. For example, acute rejection may be mediated by T cells in which the transplant recipient's T cells become alloreactive, recognizing major histocompatibility complex (MHC) antigens on the donated organ, and promoting local immune and inflammatory responses. In some embodiments, the term “rejection” refers to chronic rejection. Chronic rejection refers to a long-term loss of function in the transplant, such as due to fibrosis of the blood vessels surrounding or within the transplanted tissue. In some embodiments, acute rejection leads to chronic rejection. In some embodiments, diagnosis of acute rejection may lead to appropriate therapies that treat the acute rejection and prevent chronic rejection from occurring.

The term “transplant” is used in the broadest sense and refers to any cell, tissue, organ, or portion thereof. The term “graft” may also be used to describe a portion of a tissue or organ used as a transplant. In some embodiments, a transplant is a cell, tissue, organ, or portion thereof obtained from a donor and transplanted into a subject. The donor and the subject may be from the same or different species. A transplant from a donor of one species to a subject of another species is referred to as a “xenogeneic” transplant. In some embodiments, the transplant is an allogeneic transplant. The term “allogeneic” refers to a transplant obtained from a donor and transplanted into the subject, wherein the donor and the subject are genetically different. In some embodiments, the transplant is a syngeneic transplant. The term “syngeneic” refers to a transplant wherein the donor and the subject are genetically identical (i.e. identical twins, identical triplets, etc.)

As used herein, the terms “treat,” “treatment,” and “treating” refer to reducing the amount or severity of a particular condition, disease state (e.g., transplant rejection), or symptoms thereof, in a subject presently experiencing or afflicted with the condition or disease state. The terms do not necessarily indicate complete treatment (e.g., total elimination of the condition, disease, or symptoms thereof).

As used herein, the terms “prevent,” “prevention,” and preventing” refer to reducing the likelihood of a particular condition or disease state (e.g., transplant rejection) from occurring in a subject not presently experiencing or afflicted with the condition or disease state. The terms do not necessarily indicate complete or absolute prevention. For example “preventing transplant rejection” refers to reducing the likelihood of transplant rejection occurring in a subject not presently experiencing transplant rejection. In order to “prevent transplant rejection” a composition or method need only reduce the likelihood of transplant rejection, not completely block any possibility thereof. In some embodiments, “preventing transplant rejection” comprises preventing chronic transplant rejection in a subject currently experiencing or afflicted with acute transplant rejection. In some embodiments, “preventing transplant rejection” comprises preventing acute transplant rejection from occurring in the subject.

The terms “subject” and “patient” are used interchangeably herein and refer to any animal. In some embodiments, the subject is a mammal, including, but not limited to, mammals of the order Rodentia, such as mice and hamsters, and mammals of the order Logomorpha, such as rabbits, mammals from the order Carnivora, including Felines (cats) and Canines (dogs). mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human In some aspects, the human is an adult aged 18 years or older. In some aspects, the human is a child aged 17 years or less. In exemplary aspects, the subject bas received a transplant.

### DETAILED DESCRIPTION

In solid organ transplantation, strategies for monitoring graft rejection are limited. As there is no assay to predict the risk of solid organ transplant rejection, clinicians rely on graft biopsies and aggressive immunosuppression. Immunosuppression protects grafts from rejection but increases systemic toxicities. Invasive graft biopsies suffer from variability and are a lagging indicator of rejection. Accordingly, a minimally-invasive surveillance method is urgently needed to quantify early risk of rejection and personalize immune suppression to reduce toxicities and prevent graft injury. The synthetic scaffolds and methods described herein address this need. In some aspects, provided herein are microporous scaffold implants that accumulate immune cells, producing biomarkers of acute cellular allograft rejection (ACAR) as an engineered immunological niche. These implanted scaffold are shown herein to identify a novel gene biomarker panel that distinguishes transplant rejection vs. healthy grafts. The implantable scaffold enables remote evaluation of the early risk of rejection to reduce the frequency of routine graft biopsy and personalize immunosuppression that could prolong transplant life while minimizing patient risk.

In some aspects, provided herein are methods. In some embodiments, the methods comprise obtaining a sample from a microenvironment of a synthetic scaffold implanted in a subject that has received a transplant, and measuring an expression level of at least one RNA, at least one gene, at least one cell type, and/or at least one protein in the sample.

In some aspects, provided herein are methods for monitoring a subject. In some embodiments, provided herein are methods for monitoring transplant rejection in a subject. The term “monitoring” when used in reference to transplant rejection in the subject is used in the broadest sense and refers to any means of monitoring the patient for signs of transplant rejection, including acute and chronic transplant rejection. In some embodiments, “monitoring” refers to determining transplant rejection status in the subject. “Determining transplant rejection status” is used in the broadest sense and refers to determining whether a subject is experiencing or at risk of experiencing transplant rejection, including determining the stage of transplant rejection. In some embodiments, “monitoring transplant rejection” or “determining transplant rejection status” in the subject refers to determining/predicting whether the subject is at risk of transplant rejection. In some embodiments, “monitoring transplant rejection” or “determining transplant rejection status” refers to determining/identifying that the subject is currently experiencing transplant rejection. For example, “monitoring transplant rejection” or “determining transplant rejection status” in the subject may comprise diagnosing the subject as currently afflicted with early stages of acute transplant rejection. In some embodiments, “monitoring transplant rejection” or “determining transplant rejection status” refers to providing an early diagnosis that the subject is at risk of or is currently experiencing transplant rejection, prior to the onset of more severe transplant rejection symptoms. Accordingly, in some embodiments the methods described herein may be useful for diagnosing a subject as having or at risk of having transplant rejection, such that early medical intervention may be provided to the subject. In some embodiments, “monitoring transplant rejection” or “determining transplant rejection status” refers to determining the stage of transplant rejection a subject is experiencing. For example, “determining transplant rejection status” may involve determining that the subject is in the early stages of transplant rejection (also referred to herein as pre-symptomatic), the mid-stage of transplant rejection (also referred to herein as mid-symptomatic rejection), or the late stage of transplant rejection. In some embodiments, the methods described herein facilitate early diagnosis of transplant rejection in a subject, even when the subject has received one or more anti-rejection therapies (e.g. immunosuppressants and/or antibodies). In some embodiments, the methods described herein may be useful for monitoring an innate, systemic rejection response.

For example, in some embodiments the methods comprise detection biomarkers that are indicative of an innate, systemic rejection response rather than a local T-cell response.

In some embodiments, the subject has received a transplant and has not yet exhibited signs or symptoms of acute and/or chronic transplant rejection. In some embodiments, the subject has received a transplant and has received one or more anti-rejection therapies. In some embodiments, the subject has received a transplant and has not yet received anti-rejection therapy. In some embodiments, “monitoring transplant rejection” comprises monitoring the subject who has not yet received a given anti-rejection therapy to determine whether the anti-rejection therapy is required in the subject. In some embodiments, the subject has received a given anti-rejection therapy and “monitoring transplant rejection” comprises monitoring the subject to determine whether the anti-rejection therapy has been effective. For example, “monitoring transplant rejection” may comprise determining whether the anti-rejection therapy has successfully treated or prevented transplant rejection in the subject. For example, in some embodiments a subject that has already received a first anti-rejection therapy is determined to have or be at risk of transplant rejection, in which case the subject may require a higher dose of the anti-rejection therapy and/or another type of anti-rejection therapy. As another example, in some embodiments a subject that has already received a first anti-rejection therapy may be determined as not experiencing transplant rejection, in which case the first anti-rejection therapy is determined to have been effective at treating and/or preventing transplant rejection. In some embodiments, the first anti-rejection therapy may be maintained, the dose may be reduced, or the dose may be ceased.

Suitable anti-rejection therapies include, for example, immunosuppressive agents. Suitable immunosuppressive agents include, for example, corticosteroids (e.g. prednisolone, hydrocortisone), calcineurin inhibitors (e.g. Ciclosporin, Tacrolimus), anti-proliferative agents (e.g. Azathioprine, Mycophenolic acid), mTOR inhibitors (e g. Sirolimus, Everolimus), and the like. Additional suitable anti-rejection therapies include antibody-based treatments, including monoclonal antibodies such as anti-IL-2Rα antibodies (e.g. Basiliximab, Daclizumab), anti-IL-6R antibodies (Tocilizumab), anti-CD20 antibodies (Rituximab), and polyclonal antibodies such as polyclonal anti-T-cell antibodies (e.g. anti-thymocyte globulin, anti-lymphocyte globulin), and the like.

The subject may have received any type of transplant. In some embodiments, the gene panels described herein can be used to determine transplant rejection status in the subject, regardless of the type of transplant the subject has received. In some embodiments, the transplant comprises an organ transplant (e.g a transplant of an organ including kidney, liver, heart, lungs, skin, pancreas, trachea, intestines, or a portion thereof). In some embodiments, the transplant comprises a tissue transplant (e.g. bones, tendons, ligaments, valves, blood vessels, corneas, vascular tissues, etc.). In some embodiments, the transplant comprises a nerve tissue transplant (e g. a nerve allograft). For example, in some embodiments the transplant comprises a sensory nerve transplant, a motor nerve transplant, or mixed nerve transplant comprising both sensory and motor nerve fibers. In some embodiments, the transplant comprises a spinal cord transplant In some embodiment, the transplant comprises a transplant for central or peripheral nervous system injury, including transplantation of Schwann cells, neural stem cells, neural progenitor cells, oligodendrocyte precursor cells, mesenchymal stem cells, and the like In some embodiments, the transplant comprises a vascular composite allograft (VCA). A VCA is a transplantation of multiple composite tissues including skin, muscle, bone, and nerves.

In some embodiments, the methods comprise measuring expression level of nucleic acid (e.g. DNA, RNA) and/or protein in a sample. In some embodiments, the methods comprise measuring expression level of one or more genes in a sample. For example, the methods may comprise measuring a level of RNA (e.g. mRNA) encoding a gene. In some embodiments, the methods comprise measuring expression level of one or more proteins in a sample. In some embodiments, the methods comprise an analyzing cells from the sample. For example, analyzing cells may comprise assessing cell types (e.g. cell sub-populations) present within the sample.

For example, in some embodiments the methods comprise determining an amount of one or more cell types in the sample.

In some embodiments, the sample is obtained from a scaffold implanted in the subject. In some embodiments, the scaffold is implanted in the subject prior to a transplant in the subject. In some embodiments, the scaffold is implanted in the subject at the time of a transplant in the subject. In some embodiments, the scaffold is implanted in the subject following a transplant in in the subject.

The terms “scaffold”, “biomaterial scaffold, and “synthetic scaffold” are used interchangeably herein and refer to any scaffold which is implanted in the subject prior to, during, or after transplantation and subsequently used to collect a sample from the subject. Suitable scaffolds are described in U.S. Patent Publication No. 2020/0323893A1 and U.S. Patent Publication No. 2021/0382050, the entire contents of which are incorporated herein by reference.

In some embodiments, the scaffold is porous and/or permeable. In some embodiments, the scaffold comprises a polymeric matrix. In some embodiments, the scaffold acts as a substrate permissible for inflammation due to, for example, transplant rejection. In some embodiments, the scaffold provides an environment for attachment, incorporation, adhesion, encapsulation, etc. of agents (e.g., DNA, lentivirus, protein, cells, etc.) that create a capture site within the scaffold. In some embodiments, agents are released (e.g., controlled or sustained release) to attract circulating cells or molecules indicative of transplant rejection.

With regard to agents (e.g., therapeutic agents) and sustained release, for long term therapy (e.g., days, weeks or months) and/or to maintain the highest possible drug concentration at a particular location in the body, in some embodiments a sustained release depot formulation with the following non-limiting characteristics may be employed: (1) the process used to prepare the matrix does not chemically or physically damage the agent; (2) the matrix maintains the stability of the agent against denaturation or other metabolic conversion by protection within the matrix until release, which is important for very long sustained release; (3) the entrapped agent is released from the hydrogel composition at a substantially uniform rate, following a kinetic profile, and furthermore, a particular agent can be prepared with two or more kinetic profiles, for example, to provide in certain embodiments, a loading dose and then a sustained release dose; (4) the desired release profile can be selected by varying the components and the process by which the matrix is prepared; and (5) the matrix is nontoxic and degradable. PEG scaffolds as disclosed herein are also contemplated to function as a scaffold that achieves sustained release of a therapeutically active agent. Accordingly, in some embodiments an agent is configured for specific release rates. In further embodiments, multiple different agents are configured for different release rates. For example, a first agent may release over a period of hours while a second agent releases over a longer period of time (e.g., days, weeks, months, etc.). In some embodiments, and as described above, the scaffold or a portion thereof is configured for sustained release of agents. In some embodiments, the sustained release provides release of biologically active amounts of the agent over a period of at least 30 days (e.g., 40 days, 50 days, 60 days, 70 days, 80 days, 90 days, 100 days, 180 days, etc.).

In some embodiments, the scaffold is partially or exclusively composed of a micro-porous poly (lactide-co-glycolide) (PLG) biomaterial. In some embodiments, the scaffold is partially or exclusively composed of a micro-porous poly (e-caprolactone) (PCL), forming a PCL scaffold. Such PCL scaffolds may have a greater stability than the micro-porous poly (lactide-co-glycolide) (PLG) biomaterial scaffolds. In exemplary embodiments, the scaffold comprises PCL and/or PEG and/or alginate and/or PLG. In some embodiments, the scaffold is formed partially or exclusively of hydrogel. For example, the scaffold may be formed partially or exclusively of hydrogel, e.g., a poly (ethylene glycol) (PEG) hydrogel, to form a PEG scaffold. In some embodiments, the scaffold is a controlled release PEG scaffold. Any PEG is contemplated for use in the compositions and methods of the disclosure. In general, the PEG has an average molecular weight of at least about 5,000 daltons. In some embodiments, the PEG has an average molecular weight of at least 10,000 daltons. In some embodiments, the PEG has an average molecular weight of at least 15,000 daltons. IN some embodiments, the PEG has an average molecular weight between 5,000 and 20,000 daltons, or between 15,000 and 20,000 daltons. In some embodiments, the PEG has an average molecular weight of 5,000, of 6,000, of 7,000, of 8,000, of 9,000, of 10,000, of 11,000, of 12,000 of 13,000, of 14,000, of 15,000, of 16,000, or 17,000, or 18,000, or 19,000, of 20,000, of 21,000, of 22,000, of 23,000, or 24,000, or of 25,000 daltons. In some embodiments, the PEG is a four-arm PEG. In some embodiments, each arm of the four-arm PEG is terminated in an acrylate, a vinyl sulfone, or a maleimide. It is contemplated that use of vinyl sulfone or maleimide in the PEG scaffold renders the scaffold resistant to degradation. It is further contemplated that use of acrylate in the PEG scaffold renders the scaffold susceptible to degradation.

In some embodiments, one or more agents are associated with a scaffold. For example, agents may be associated with the scaffold to establish a hospitable environment for markers of transplant rejection (e.g. inflammation). As another example, one or more agents may be associated with a scaffold to provide a therapeutic benefit to a subject. Agents may be associated with the scaffold by covalent or non-covalent interactions, adhesion, encapsulation, etc. In some embodiments, a scaffold comprises one or more agents adhered to, adsorbed on, encapsulated within, and/or contained throughout the scaffold. The present invention is not limited by the nature of the agents. Such agents include, but are not limited to, peptides, proteins, nucleic acid molecules, small molecule drugs, lipids, carbohydrates, cells, cell components, and the like. In various embodiments, the agent is a therapeutic agent. In some embodiments, two or more (e.g., 3, 4, 5, 6, 7, 8, 9, 10 . . . 20 . . . 30 . . . 40 . . . , 50, amounts therein, or more) different agents are included on or within the scaffold. In other embodiments, no agents are provided with the scaffold. In some aspects, the scaffold is modified to deliver proteins, peptides, small molecules, gene therapies, biologics, etc. to enhance the signal to noise ratio.

In some embodiments, the scaffold comprises a polymeric matrix. In some embodiments, the matrix is prepared by a gas foaming/particulate leaching procedure, and includes a wet granulation step prior to gas foaming that allows for a homogeneous mixture of porogen and polymer and for sculpting the scaffold into the desired shape. In some embodiments, scaffolds may be formed of a biodegradable polymer, e.g., PCL, that is fabricated by emulsifying and homogenizing a solution of polymer to create microspheres. Other methods of microsphere production are known in the art and are contemplated by the present disclosure. See, e.g., U.S. Patent Application Publication Numbers 2015/0190485 and 2015/0283218, each of which is incorporated herein in its entirety. The microspheres are then collected and mixed with a porogen (e.g., salt particles), and the mixture is then pressed under pressure. The resulting discs are heated, optionally followed by gas foaming. Finally, the salt particles are removed. The fabrication provides a mechanically stable scaffold which does not compress or collapse after in vivo implantation, thus providing proper conditions for cell growth. In some aspects, the scaffolds are formed of a substantially non-degradable polymer, e.g., PEG. Degradable hydrogels encapsulating gelatin microspheres may be formed based on a previously described Michael-Type addition PEG hydrogel system with modifications [Shepard et al., Biotechnol Bioeng. 109 (3): 830-9 (2012)]. Briefly, four-arm polyethylene glycol) vinyl sulfone (PEG-VS) (20 kDa) is dissolved in 0.3 M triethanolamine (TEA) pH 8.0 at a concentration of 0.5 mg/L to yield a final PEG concentration of 10%. The plasmin-degradable trifunctional (3 cysteine groups) peptide crosslinker (Ac-GCYKN CGYKN CG) is dissolved in 0.3 M TEA pH 10.0 to maintain reduction of the free thiols at a concentration that maintain a stoichiometrically balanced molar ratio of VS: SH. Prior to gelation, gelatin microspheres are hydrated with 10 μl sterile Millipore or lentivirus solution. Subsequently, the PEG and peptide crosslinking solutions are mixed well and immediately added to the hydrated gelatin microspheres for encapsulation. In some embodiments, and as described above, salt is used as the porogen instead of gelatin microspheres. In this case, the PEG solution is made in a saturated salt solution, so that the porogen does not significantly dissolve.

In some embodiments, UV crosslinking is used instead of peptide crosslinking. Ultraviolet crosslinking is contemplated for use with PEG-maleimide, PEG-VS, and PEG-acrylate.

Scaffolds of the present disclosure may comprise any of a large variety of structures including, but not limited to, particles, beads, polymers, surfaces, implants, matrices, etc. Scaffolds may be of any suitable shape, for example, spherical, generally spherical (e.g., all dimensions within 25% of spherical), ellipsoidal, rod-shaped, globular, polyhedral, etc. The scaffold may also be of an irregular or branched shape.

In some embodiments, a scaffold comprises nanoparticles or microparticles (e.g., compressed or otherwise fashioned into a scaffold). In various embodiments, the largest cross-sectional diameters of a particle within a scaffold is less than about 1,000 μm, 500 μm, 200 μm, 100 μm, 50 μm, 20 μm, 10 μm, 5 μm, 2 μm, 1 μm, 500 nm, 400 nm, 300 nm, 200 nm or 100 nm. In some embodiments, a population of particles has an average diameter of: 200-1000 nm, 300-900 nm, 400-800 nm, 500-700 nm, etc. In some embodiments, the overall weights of the particles are less than about 10,000 kDa, less than about 5,000 kDa, or less than about 1,000 kDa, 500 kDa, 400 kDa, 300 kDa, 200 kDa, 100 kDa, 50 kDa, 20 kDa, 10 kDa.

In some embodiments, a scaffold comprises PCL. In further embodiments, a scaffold comprises PEG. In certain embodiments, PCL and/or PEG polymers and/or alginate polymers are terminated by a functional group of chemical moiety (e.g., ester-terminated, acid-terminated, etc.).

In some embodiments, the charge of a matrix material (e.g., positive, negative, neutral) is selected to impart application-specific benefits (e.g., physiological compatibility, beneficial interactions with chemical and/or biological agents, etc.). In certain embodiments scaffolds are capable of being conjugated, either directly or indirectly, to a chemical or biological agent). In some instances, a carrier has multiple binding sites (e.g., 2, 3, 4, 5, 6, 7, 8, 9, 10 . . . 20 . . . 50 . . . 100, 200, 500, 1000, 2000, 5000, 10,000, or more).

In some embodiments, the lifetimes of the scaffolds are well within the timeframe of clinical significance are demonstrated. For example, stability lifetimes of greater than 90 days are contemplated, with percent degradation profiles of less than about 50%, 45%, 40%, 35%, 30%, 25%, 20%, 15%, 10%, 5%, and 1% respectively, where the percent degradation refers to the scaffolds' ability to maintain its structure for sufficient cell capture as a comparison of its maximum capture ability. Such ability is measured, for example, as the change in porous scaffold volume over time, the change in scaffold mass over time, and/or the change in scaffold polymer molecular weight over time. These long lifetimes mean that scaffolds can now be applied in patient-friendly conditions that allow subjects to wear the scaffold under normal daily living conditions, inside and outside the clinical environment.

In some embodiments, the scaffold or a portion thereof is configured to be sufficiently porous to permit cells and molecules of interest into the pores. The size of the pores may be selected for particular cell types of interest and/or for the amount of ingrowth desired and are, for example without limitation, at least about 20 μm, 30 μm, 40 μm, 50 μm, 100 μm, 200 μm, 500 μm, 700 μm, or 1000 μm. In some embodiments, the PEG gel is not porous but is instead characterized by a mesh size that is, e.g., 10 nanometers (nm), 15 nm, 20 nm, 25 nm, 30 nm, 40 nm, or 50 nm.

The scaffold may be implanted at any suitable location in the body of the subject. For example, the scaffold may be implanted subcutaneously. As another example, the scaffold may be implanted in a fat pad. In some embodiments, the scaffold is implanted proximal to the site of the transplant. In some embodiments, the scaffold is implanted at a separate site, away from the site of the transplant. In some embodiments, more than one scaffold is implanted in the subject. For example, at least 1, at least 2, at least 3, at least 4, at least 5, at least 6, at least 7, at least 8, at least 9, or at least 10 scaffolds may be implanted in a subject. In some embodiments, samples from each of the scaffolds implanted in the subject are obtained and expression profiles of each sample are measured.

Implantation of synthetic scaffolds in a subject (e.g. in the subcutaneous space or fat pad of a subject), trigger in vivo events (e.g., a foreign body response, an immune response against the scaffold) that result in the creation of a niche in the subject at the implantation site. In some embodiments, the sample is obtained from the niche. As used herein, the term “niche” refers to the area of cells and molecules located at or near the site at which a synthetic scaffold is or was implanted in the subject (e.g. at the scaffold implantation site). In some embodiments, the niche reflects the in vivo events caused by the implantation of the scaffold. In some aspects, the niche is physically attached to the scaffold. In some embodiments, the niche comprises cells and other molecules or factors involved in an immune response against the synthetic scaffold implanted in the subject at the implantation site. In some embodiments, the niche may be representative of the patient's health status. For example, the niche may be representative of the patient's status with regard to the transplant (e.g. whether the patient is experiencing transplant rejection). In some embodiments, the content of the niche changes as the disease (e.g. transplant rejection) changes (progresses, regresses). In some embodiments, the sample is obtained from the niche. For example, in some embodiments the niche is biopsied, and the analysis of the biopsied sample allows for the disease diagnosis and/or prognosis, in addition to treatment monitoring.

In some embodiments, the methods described herein involve measuring expression of a gene, RNA, or protein in a sample. In some embodiments, the methods comprise an analyzing cells from the sample. For example, in some embodiments the methods involve assessing cell types (e.g. cell sub-populations) present within the sample. In some embodiments, analyzing cells comprises determining an amount of at least one cell type present within the sample. In some embodiments, analyzing cells comprises determining an amount of multiple cell types present within the sample. In some embodiments, analyzing cells comprises determining a ratio of one cell type relative to another. Analyzing cells may be performed as an alternative to or in addition to measuring expression of a gene, RNA, or protein in the sample.

For the methods described herein, the sample is obtained from the scaffold microenvironment. The term “microenvironment” when used in reference to the scaffold (e.g. “scaffold microenvironment”, “microenvironment of the scaffold”, etc.) is used herein to include the scaffold, a portion of the scaffold, and the niche. In some embodiments, the sample is obtained from the scaffold or a portion thereof. In some embodiments, the sample is obtained from the niche. The sample obtained from the scaffold microenvironment may be isolated from the subject prior to use for analysis of expression of a gene, RNA, or protein in the sample.

In some embodiments, the scaffold or a portion thereof is retrieved from the subject to provide the sample from which expression is measured. For example, the scaffold or a portion thereof may be biopsied, and used to obtain the sample from which DNA/RNA/protein expression is measured and/or cells are analyzed (e.g. an amount of at least one cell type is determined) In some embodiments, the methods involve measuring a level of expression of a gene, an RNA, e.g., a messenger RNA (mRNA), or a protein, in a sample obtained from a niche at the implantation site of a synthetic scaffold. In some embodiments, the methods involve analyzing cells in a sample (e.g. determining populations of cell types present within a sample) obtained from a niche at the implantation site of a synthetic scaffold. For example, at a time point of interest (e.g. after a given period of time following transplant) a biopsy may be performed to remove the scaffold or a portion thereof. For example, a biopsy may be performed to remove the entire scaffold. Alternatively, a core-needle biopsy may be performed to remove a portion of the scaffold microenvironment (e.g. a portion of the scaffold or a portion of the niche).

With the scaffold or a portion thereof removed, or a sample of the niche of the scaffold removed, the contents may be processed for molecular content determination. In some examples, such processing includes isolating RNA and/or protein (e.g., by homogenization in Trizol reagent or detergent, respectively). In some embodiments, the contents of the sample are processed to analyze cells contained within the sample. For example, cell populations may be assessed by techniques including fluorescent-assisted cell sorting (FACS), magnetic-assisted cell sorting, and/or histological techniques including immunohistochemistry or fluorescent imaging technologies. Optionally, in some examples, cell populations within the scaffold may be fractionated. For example, cell populations may be fractionated for cell type specific analysis. Suitable fractionation techniques include methods that separate cell populations based on fluorescent-assisted cell sorting or magnetic-assisted cell sorting.

In some embodiments, RNA and/or protein is isolated from the scaffold or portion thereof or from the niche, and used for analysis of gene or protein expression. Analysis of gene or protein expression may be achieved, in some examples, using either qRT-PCR or RNAseq, for gene expression, and either ELISA or Luminex (bead-based multiplex assays), for protein expression. In some embodiments, the methods comprise measuring a combination of at least two of an expression level of a gene, an RNA, and a protein. In some embodiments, the methods comprise measuring the expression level of at least one gene, at least one RNA, and at least one protein. In some embodiments, the methods comprise measuring the expression level of a plurality of different genes, a plurality of RNA, and/or a plurality of proteins. In some embodiments, the methods comprise measuring the expression level of at least 2, 3, 4, 5 or more genes, at least 2, 3, 4, 5 or more RNA, and/or at least 2, 3, 4, 5 or more proteins in the sample. In some embodiments, the methods comprise measuring the expression level of at least 10, 15, 20 or more genes, at least 10, 15, 20 or more RNA, and/or at least 10, 15, 20 or more proteins in the sample. In some embodiments, the methods comprise measuring the expression level of at least 50,100, 200 or more genes, at least 50, 100, 200 or more RNA, and/or at least 50, 100, 200 or more proteins in the sample.

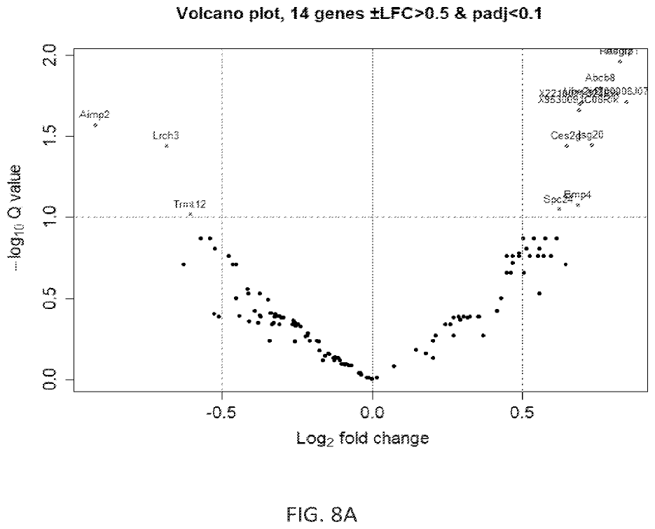

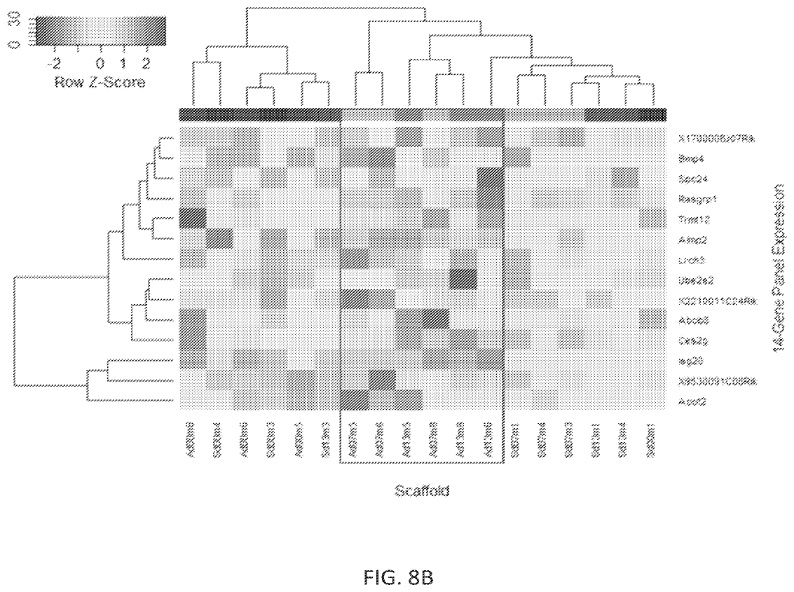

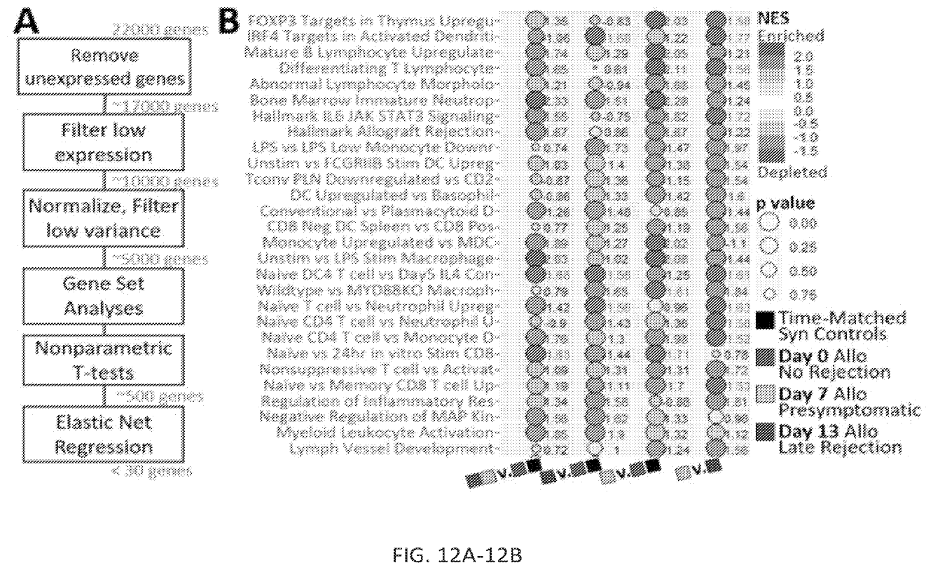

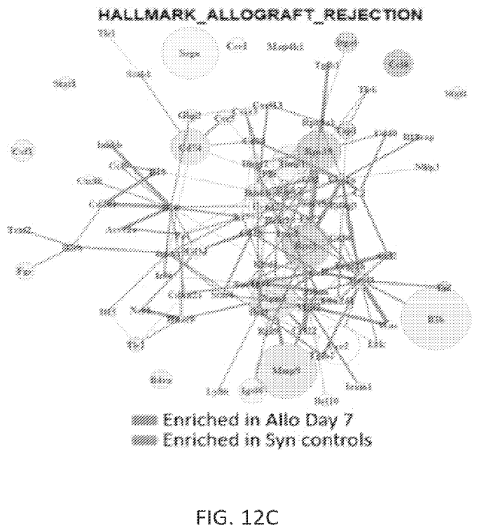

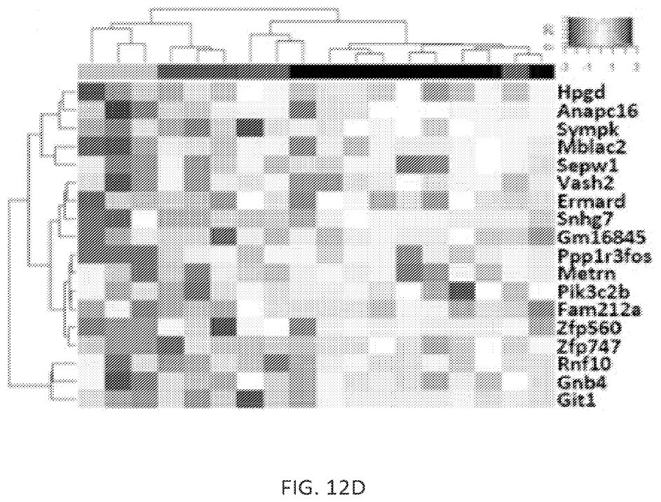

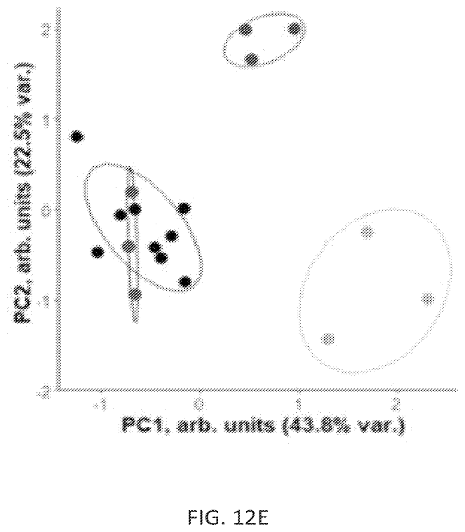

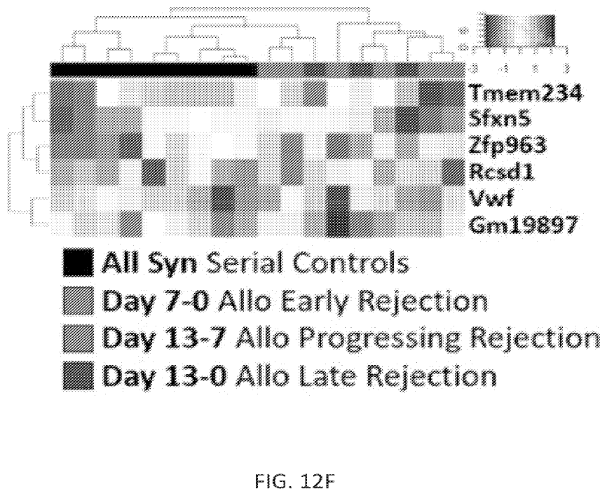

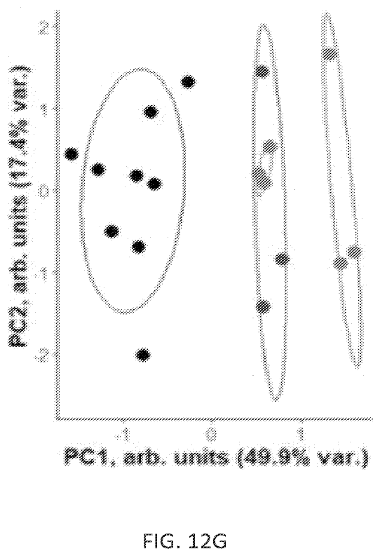

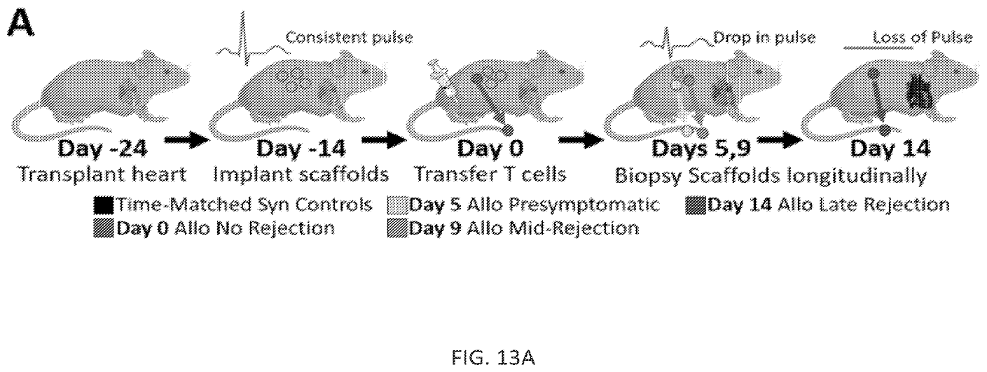

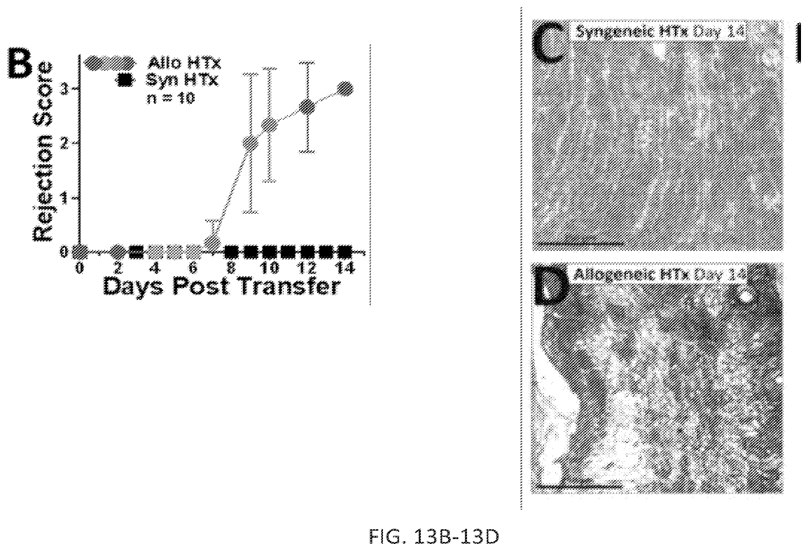

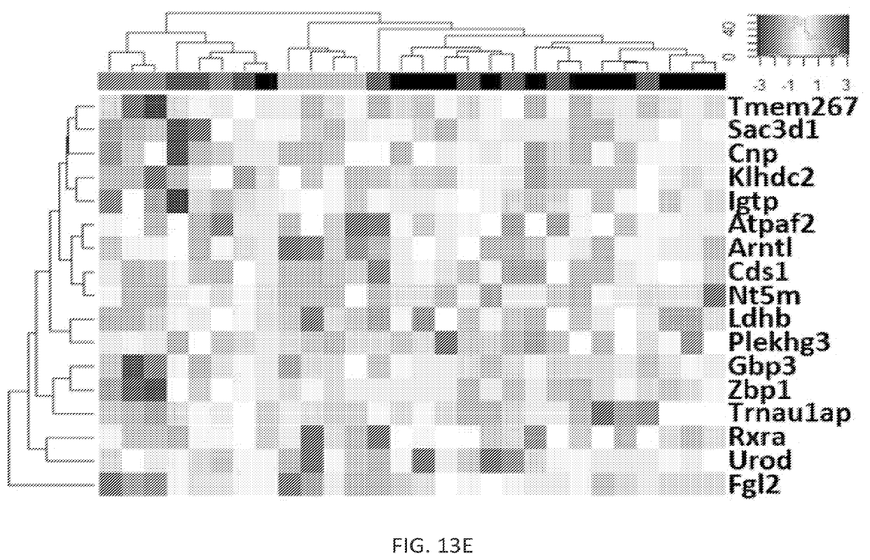

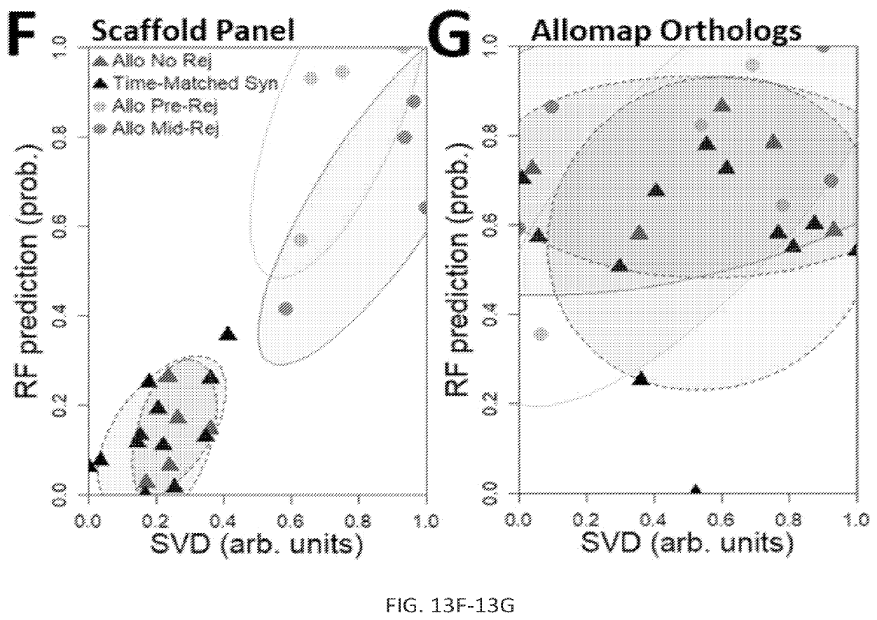

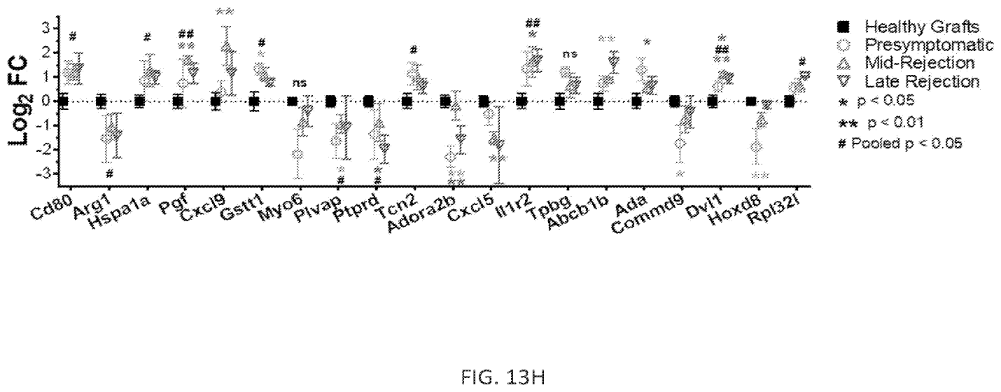

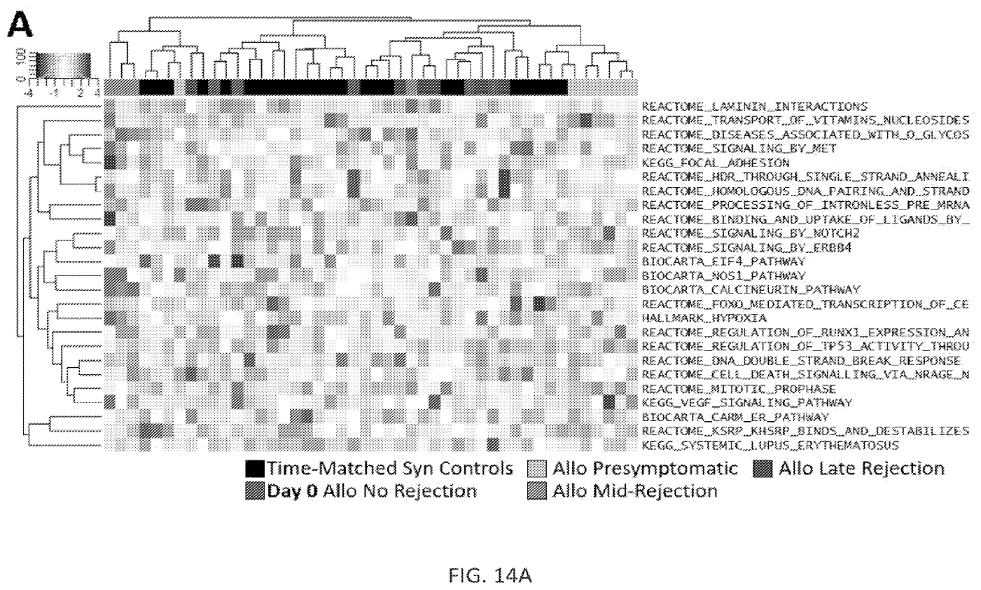

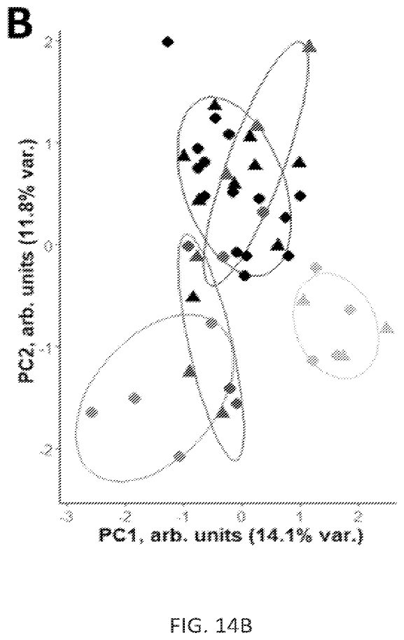

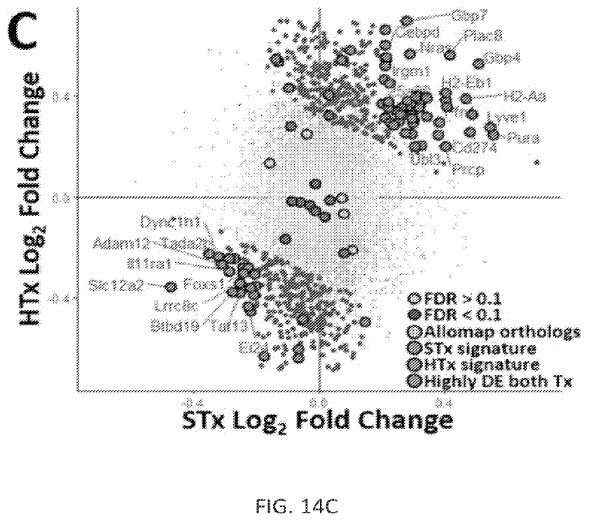

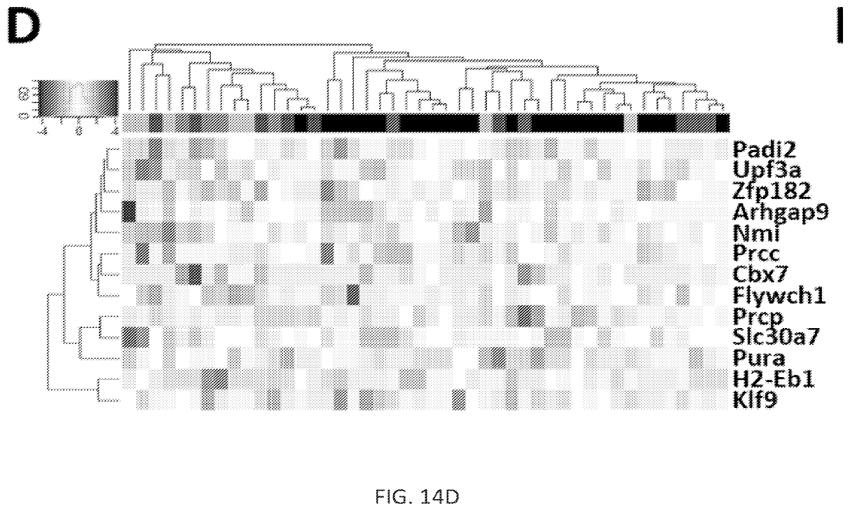

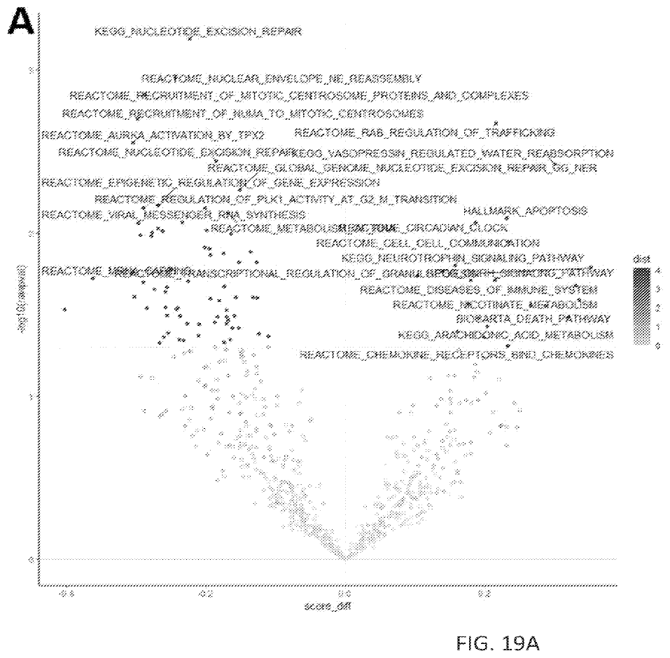

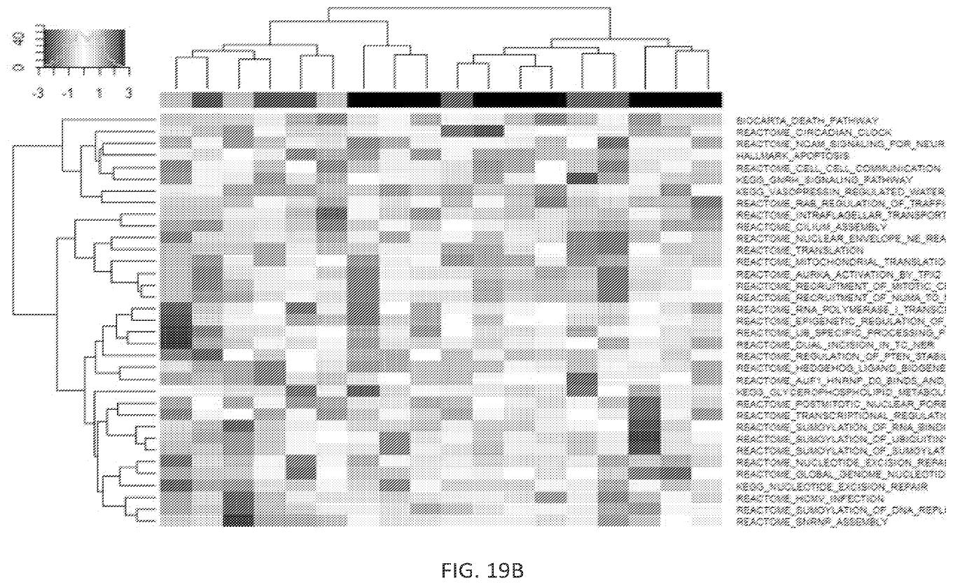

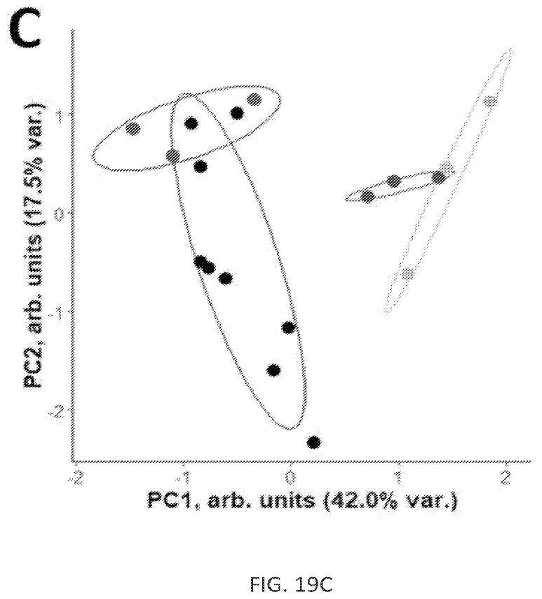

In some embodiments, the methods comprise measuring the expression level of a panel of genes. In some embodiments, the panel comprises at least three genes. In some embodiments, the panel comprises 3-50 genes. In some embodiments, the panel comprises 6-25 genes. In some embodiments, the methods comprise measuring the expression level of a panel of genes shown in FIG. 8B, FIG. 12D, FIG. 12F, FIG. 13E, FIG. 14D, or FIG. 19B. In some embodiments, the methods comprise measuring the expression level of a panel of genes shown in FIG. 12D, FIG. 12F. FIG. 13E, or FIG. 14D. In some embodiments, the methods comprise determining transplant rejection status in the subject based upon the expression level of the panel of genes, such as based upon whether the expression level or one or more genes in the panel is increased or decreased compared to a reference value In some embodiments, the methods comprise determining transplant rejection status in the subject based upon the expression level of the panel of genes in accordance with the data presented in FIG. 8B, FIG. 12D, FIG. 12F, FIG. 13E, FIG. 14D, or FIG. 19B. In some embodiments, the methods comprise measuring the expression level of more than 10 different genes, more than 100 different genes, more than 1000 different genes, or more than 5000-10,000 different genes, and the expression levels of the different genes constitute a gene signature.

In some embodiments, the methods comprise measuring the expression level of more than 10 different RNA, more than 100 different RNA, more than 1000 different RNA, more than S000-10,000 different RNA, and the expression levels of the different RNA constitute an RNA signature, or a transcriptome.

In some embodiments, the method comprises measuring an expression level of one or more genes selected from X1700008J07Rik, bone morphogenic protein 4 (Bmp4), SPC24 Component Of NDC80 Kinetochore Complex (Spc24), RAS Guanyl Releasing Protein 1 (Rasgrp1), TRNA Methyltransferase 12 Homolog (Trmt12), Aminoacyl TRNA Synthetase Complex Interacting Multifunctional Protein 2 (Aimp2), Leucine Rich Repeats And Calponin Homology Domain Containing 3 (Lrch3), Ubiquitin Conjugating Enzyme E2 E2 (Ube2e2), X2210011C24Rik, ATP Binding Cassette Subfamily B Member 8 (Abcb8), carboxylesterase 2G (Ces2g), Interferon Stimulated Exonuclease Gene 20 (Isg20), X950091C08Rik, and Acyl-CoA Thioesterase 2 (Acot2).

In some embodiments, the method comprises measuring an expression level of on or more genes identified in Table 1. In some embodiments, the method comprises measuring an expression level of one or more genes identified in Table 2. In some embodiments, the method comprises measuring an expression level of one or more genes identified in Table 3. In some embodiments, the expression level is used to monitor transplant rejection in the subject. In some embodiments, the expression level of at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, or more than 20 genes in Table 1, Table 2, or Table 3 is measured.

In some embodiments, the method comprises measuring an expression level of one or more genes selected from 15-Hydroxyprostaglandin Dehydrogenase (Hpgd), Anaphase Promoting Complex Subunit 16 (Anapc16), Symplekin Scaffold Protein (Sympk), Metallo-Beta-Lactamase Domain Containing 2 (Mblac2), Selenoprotein W (Sepw1), Vasohibin 2 (Vash2), ER Membrane Associated RNA Degradation (Ermard), Small Nucleolar RNA Host Gene 7 (Snhg7), Gm16845, protein phosphatase 1, regulatory subunit 3F, opposite strand (Ppp1r3fos), Meteorin, Glial Cell Differentiation Regulator (Metrn), Phosphatidylinositol-4-Phosphate 3-Kinase Catalytic Subunit Type 2 Beta (Pik3c2b), Family with Sequence Similarity 212, Member A (Fam212a), zinc finger protein 560 (Zfp560), zinc finger protein 747 (Zfp747), Ring Finger Protein 10 (Rnf10), G Protein Subunit Beta 4 (Gnb4), and GIT ArfGAP 1 (Git1). In some embodiments, the transplant is a skin transplant, and the method comprises measuring the expression level of one or more genes selected from Hpgd, Anapc16, Sympk, Mblac2, Sepw1, Vash2, Ermard, Snhg7, Gm16845, Ppplr3fos, Metrn, Pik3c2b, Fam212a, Zfp560, Zfp747, Rnf10, Gnb4, and Git1.

In some embodiments, the method comprises measuring an expression level of one or more genes selected from Transmembrane Protein 234 (Tmem234), sideroflexin 5 (Sfxn5), zinc finger protein 963 (Zfp963), RCSD Domain Containing 1 (Rcsd1), Von Willebrand Factor (Vwf), and Gm19897. In some embodiments, the transplant is a skin transplant, and the method comprises measuring an expression level of one or more genes selected from Tmem234, Sfxn5, Zfp963, Rcsd1, Vwf, and Gm19897.

In some embodiments, the method comprises measuring an expression level of one or more genes selected from Transmembrane Protein 267 (Tmem267), Stearoyl-CoA desaturase 1

(Sacd1), 2′,3′-Cyclic Nucleotide 3′ Phosphodiesterase (Cnp), Kelch Domain Containing 2 (Klhdc2), interferon gamma induced GTPase (Igtp), ATP Synthase Mitochondrial F1 Complex Assembly Factor 2 (Atpaf2), Basic Helix-Loop-Helix ARNT Like 1 (Arntl), CDP-Diacylglycerol Synthase 1 (Cds1), 5′,3′-Nucleotidase, Mitochondrial (Nt5m), Lactate Dehydrogenase B (Ldhb), Pleckstrin Homology And RhoGEF Domain Containing G3 (Plekhg3), Guanylate Binding Protein 3 (Gbp3), Z-DNA Binding Protein 1 (Zbp1), TRNA Selenocysteine 1 Associated Protein 1 (Trnaulap), Retinoid X Receptor Alpha (Rxra), uroporphyrinogen decarboxylase (Urod), and Fibrinogen-Like Protein 2 (Fgl2). In some embodiments, the transplant is a heart transplant, and the method comprises measuring an expression level of one or more genes selected from Tmem267, Sacd1, Cnp, Klhdc2, Igtp, Atpaf2, Arntl, Cds1, Nt5m, Ldhb, Plekhg3, Gbp3, Zbp1, Trnaulap, Rxra, Urod, and Fgl2.

In some embodiments, the method comprises measuring an expression level of one or more genes selected from Peptidyl Arginine Deiminase 2 (Padi2), Ufp3a, zinc finger protein 182 (Zfp182), Rho GTPase Activating Protein 9 (Arhgap9), N-Myc And STAT Interactor (Nmi), Proline Rich Mitotic Checkpoint Control Factor (Prcc), Chromobox 7 (Cbx7), FLYWCH-Type Zinc Finger 1 (Flywch1), Prolylcarboxypeptidase (Prcp), Solute Carrier Family 30 Member 7 (Slc30a7), Purine Rich Element Binding Protein A (Pura), histocompatibility 2, class II antigen E beta (H2-Eb1), and KLF Transcription Factor 9 (Klf9). In some embodiments, the method comprises measuring an expression level of one or more genes selected from Padi2, Ufp3a, Zfp182, Arhgap0, Nmi, Prcc, Cbx7, Flywch1, Prcp, Slc30a7, Pura, H2-Eb1, and Klf9 regardless of the type of transplant (e.g. skin, heart, lung, etc.) received by the subject.

In some embodiments, the methods of monitoring transplant status in the subject described herein comprise comparing the expression level (e.g. of the gene, RNA, of protein) in the sample to a reference level. The term “reference level” is used in the broadest sense and includes a “control level”, a “threshold” level, a “baseline level”, a “cutoff” level, and other similar terms. A control level refers to an expression level or amount determined based upon results collected from control subjects that have not received a transplant. For example, the expression level in the sample may be compared to a control level. In some aspects, the control level is that of a control subject which may be a matched control (e.g. a control of the same species, gender, ethnicity, age group, smoking status, BMI, current therapeutic regimen status, medical history, or a combination thereof as the subject), but differs from the subject being diagnosed in that the control has not received a transplant. In some embodiments, the control level is that of a control subject that has received a syngeneic transplant, whereas the subject has received an allogeneic transplant. In some embodiments, the control the control level is an expression level obtained from the subject prior to transplant. Such a control level obtained from the subject prior to transplant is also referred to as a “baseline” level. In some embodiments, the “baseline level” is measured in a sample obtained from the scaffold microenvironment of the subject prior to the subject receiving the transplant. In some embodiments, the expression level in the sample is compared to a cutoff level. In some embodiments, the cutoff level is indicative of transplant rejection. For example, if levels of expression in the sample are higher than a cutoff level that is indicative of transplant rejection, the subject may be indicated to be at risk of or experiencing transplant rejection.

In some embodiments, expression levels or amounts in the sample may be increased (e.g. relative to a control level or a cutoff level). As used herein, the term “increased” with respect to level (e.g., expression level, biological activity level) refers to any % increase above a control level. The increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

In some embodiments, expression levels or amounts in the sample may be decreased (e.g. relative to a control level or a cutoff level). As used herein, the term “decreased” with respect to level (e.g., expression level, biological activity level) refers to any % decrease below a control level. The decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

In some embodiments, an increased expression level or amount of one or more genes, RNAs, cell types, or proteins in the sample is indicative that the subject is at risk of or currently experiencing transplant rejection. In some embodiments, decreased expression level or amount of one or more genes, RNAs, cell types, or proteins in the sample is indicative that the subject is at risk of or currently experiencing transplant rejection. The change relative to a control, threshold, or cutoff level and its significance for transplant rejection may vary depending on the specific gene, protein, or RNA measured.

In some embodiments, the method comprises measuring the expression level or amount of a gene, RNA, cell type, or protein at a first time point and at a second time point, and the measured level of the first time point serves as a control level or establishes a baseline. In some embodiments, the first time point is prior to transplant and the second time point is after transplant.

In some embodiments, the levels of expression are measured and the measured levels are normalized or calibrated to a level of a housekeeping gene. Any suitable housekeeping gene may be used. In some embodiments, the housekeeping gene is one or more of GAPDH, Hmbs, Tbp, Ubc, Ywhaz, Polr2a. Suitable housekeeping genes include, for example, beta-actin (ACTB), aldolase A, fructose-bisphosphate (ALDOA), glyceraldehyde-3-phosphate dehydrogenase (GAPDH), phosphoglycerate kinase 1 (PGK1), RNA polymerase II subunit A (POLR2A), lactate dehydrogenase A (LDHA), ribosomal protein S27a (RPS27A), ribosomal protein L19 (RPL19), ribosomal protein L11 (RPL11), non-POU domain containing, octamer-binding (NONO), Rho GDP dissociation inhibitor alpha (ARHGDIA), ribosomal protein L32(RPL32), ubiquitin C (UBC), HMBS, RBP, and Ywhaz.

In some embodiments, expression levels may be measured at multiple time points. For example, a sample may be obtained from the subject (e.g. from the scaffold or a portion thereof, or from the niche) at a first time point, a second time point, a third time point, etc. In some embodiments, at least two time points are following transplant. Accordingly, the methods described herein may be used to monitor a subject across time for risk of or occurrence of transplant rejection.

In some embodiments, the expression levels of the genes, RNA, and/or proteins are processed through an algorithm to obtain a single metric or single score of gene expression, RNA expression, or protein expression In some embodiments, the expression levels are normalized to housekeeping gene expression levels. In some embodiments, the expression levels are processed through singular value decomposition, dynamic mode decomposition, principle component analysis, fisher linear discriminant, or linear combination. In some embodiments, the expression levels of the genes, RNA, and/or proteins (optionally normalized to housekeeping gene expression levels) or the single metric or single score is processed through a machine learning algorithm to obtain a score indicative of transplant status. For example, the score may be indicative of a % chance of transplant rejection. In some embodiments, the metric of gene expression, RNA expression, or protein expression is combined with the prediction score to obtain a graphical or numerical output, which may be used as a control (or a panel of controls) against which the measured levels are compared.

In some embodiments, more than one sample is obtained from the scaffold, portion thereof, or niche, and each sample is obtained at a different point in time. For example, in some embodiments, 2, 3, 4, 5, 6, 7, 8, 9, 10, or more samples are obtained, each sample obtained at a different point in time. In some embodiments, a sample is obtained once a day, 2× per day, 3× per day, 4× per day or more frequently. In some embodiments, a sample is obtained every 2, 3, 4, 5, or 6 days In some embodiments, a sample is obtained once a week. In some embodiments, a sample is obtained once every 2 weeks, 3 weeks, 4 weeks, 5 weeks, 6 weeks, 7 weeks, or 8 weeks or less frequently In some embodiments, a sample is obtained on a regular basis based on the analysis of a first sample. In some embodiments, a sample is obtained on a regular basis until a pre-determined goal is met. In some embodiments, the pre-determined goal is the determination of the subject as exhibiting a complete therapeutic response to a treatment (e.g. a complete therapeutic response to a therapy for transplant rejection).

In some embodiments, at least one sample is obtained from the subject (e.g from the scaffold, from a portion of the scaffold, or from the niche) one day or more following transplant in the subject. For example, in some embodiments at least one sample is obtained about 24 hours, about 36 hours, about 48 hours, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, or more than 3 weeks following transplant (e g. about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks, etc.).

In some embodiments, at least one sample is obtained at a first time point, and at least one sample is obtained at a second time point that is after the first time point. In some embodiments, the methods comprise determining that the subject is at risk of or is currently experiencing transplant rejection if the expression level or amount of one or more genes, RNA, cell types, or protein is changed from the first time point to the second time point (e.g. increases or decreases). For example, the subject may be at risk of or experiencing transplant rejection if the expression level or amount of one or more genes, RNA, cell types, or proteins is increased by at least a threshold amount from the first time point to the second time point. As another example, the subject may be at risk of or experiencing transplant rejection if the expression level or amount or one or more genes, RNA, cell types, or proteins is decreased by at least a threshold amount from the first time point to the second time point. In some embodiments, a first sample is collected within 24 hours to 7 days after transplant, and a second sample is obtained from the subject within 7-14 days after transplant As another example, in some embodiments a first sample is collected within 24 hours after transplant, and a second sample is collected within 48 hours—14 days after transplant. In some embodiments, a first sample is obtained within 7 days (e.g. about 12 hours, about 24 hours, about 2 days, about 3 days, about 4 days, about 5 days, about 6 days, or about 7 days) after transplant and the second sample is obtained at least 24 hours after the first sample. In some embodiments, the first sample is obtained within 7 days after transplant, and the second sample is obtained 1-14 days (e.g about 1 day, about 2 days, about 3 days, about 4 days, about 5 days, about 6 days, about 7 days, about 8 days, about 9 days, about 10 days, about 11 days, about 12 days, about 13 days, about 14 days) after the first sample. In some embodiments, the first sample is obtained within 7 days after transplant, and the second sample is obtained 1-7 days after the first sample. In some embodiments, the first sample is obtained within S days (e g. about 12 hours, about 24 hours, about 2 days, about 3 days, about 4 days, about 5 days) after transplant, and the second sample is obtained at least 24 hours after the first sample. In some embodiments, the first sample is obtained within 5 days after transplant, and the second sample is obtained 1-14 days after the first sample. In some embodiments, the first sample is obtained within 5 days after transplant, and the second sample is obtained 1-7 days after the first sample. In some embodiments, the method comprises determining that the subject is at risk of or is currently experiencing rejection if the expression level or amount of one or more genes, RNA, cell types, and/or proteins are increased or decreased from the first time point to the second time point.

In some embodiments, the method comprises measuring expression level of one or more genes selected from X1700008J07Rik, Bmp4, Spc24, Rasgrp1, Trmt12, Aimp2, Lrch3, Ube2e2, X2210011C24Rik, Abeb8, Ces2g, Isg20, X950091C08Rik, and Acot2. In some embodiments, the method comprises measuring expression level of at least 2, at least 3, at least 4, at least 5, at least 6, at least 7, at least 8, at least 9, at least 10, at least 11, at least 12, at least 13, or at least 14 genes selected from X1700008J07Rik, Bmp4, Spc24, Rasgrp1, Trmt12, Aimp2, Lrch3, Ube2e2, X2210011C24Rik, Abcb8, Ces2g, Isg20, X950091C08Rik, Acot2 In some embodiments, the method further comprises monitoring transplant rejection in the subject based upon the levels of expression of the one or more genes. For example, the method may comprise determining that the subject is at risk of or is currently experiencing transplant rejection if expression level of one or more genes in the sample is increased relative to a reference level (e.g. control level or cutoff level). For example, in some embodiments the method comprises determining that the subject is at risk of or is currently experiencing transplant rejection if the expression level of X1700008J07Rik, Bmp4, Spc24, Rasgrp1, Ube2e2, X2210011C24Rik, Abcb8, Ces2g, Isg20, X950091C08Rik, and/or Acot2 is increased relative to a control level. The control level may be a level measured in a control subject that has not received a transplant. The control level may be measured in a control subject that received a syngeneic transplant.

In some embodiments, the method comprises determining that the subject is at risk of or is currently experiencing transplant rejection if the expression level of one or more genes in the sample is decreased relative to a reference level (e.g. control level or cutoff level). For example, the method may comprise determining that the subject is at risk of or is currently experiencing transplant rejection if the expression level of Aimp2, Lrch3, and/or Trmt12 is decreased relative to a control level. The control level may be a level measured in a control subject that bas not received a transplant. The control level may be measured in a control subject that received a syngeneic transplant.

In some embodiments, the method comprises determining that the subject is at risk of or is currently experiencing transplant rejection if the expression level of one or more genes selected from X1700008J07Rik, Bmp4, Spc24, Rasgrp1, Ube2e2, X2210011C24Rik, Abcb8, Ces2g, Isg20, X950091C08Rik, and Acot2 is increased relative to a reference level (e.g. control level or cutoff level) and the expression level of one or more genes selected from Aimp2, Lrch3, and Trmt12 is decreased relative to a reference level (e.g. control level or a cutoff level).

In some embodiments, the method comprises measuring expression level of one or more genes selected from X1700008J07Rik, Bmp4, Spc24, Rasgrp1, Trmt12, Aimp2, Lrch3, Ube2e2, X2210011C24Rik, Abcb8, Ces2g, Isg20, X950091C08Rik, Acot2 at a first time point following transplant, and measuring expression level of the one or more genes at a second time point after the first time point. In some embodiments, the subject is determined to be at risk of or currently experiencing transplant rejection if the expression level of at least one of the one or more genes is increased at the second time point compared to the first time point. For example, the subject may be determined to be at risk of or currently experiencing transplant rejection if the expression level of X1700008J07Rik, Bmp4, Spc24, Rasgrp1, Ube2e2, X2210011C24Rik, Abcb8, Ces2g, Isg20, X950091C08Rik, and/or Acot2 is increased (e.g. increased by at least a threshold amount or percentage) at the second time point compared to the first time point. In some embodiments, the subject is determined to be at risk of or currently experiencing transplant rejection if the expression level of the one or more genes is decreased at the second time point compared to the first time point. For example, the subject may be determined to be at risk of or currently experiencing transplant rejection if the expression level of Aimp2, Lrch3, and/or Trmt12 is decreased (e.g. decreased by at least a threshold amount or percentage) at the second time point compared to the first time point In some embodiments, the subject is determined to be at risk of or currently experiencing transplant rejection if the expression level of X1700008J07Rik, Bmp4, Spc24, Rasgrp1, Ube2e2, X2210011C24Rik, Abcb8, Ces2g, Isg20, X950091C08Rik, and/or Acot2 is increased and the expression level of Aimp2, Lrch3, and/or Trmt 12 is decreased at the second time point compared to the first time point.

In some embodiments, the method comprises measuring an expression level of one or more genes selected Hpgd, Anapc16, Sympk, Mblac2, Sepw1, Vash2, Ermard, Snhg7, Gm16845, Ppp1r3fos, Metrn, Pik3c2b, Fam212a, Zfp560, Zfp747, Rnf10, Gnb4, and Git1. In some embodiments, the method comprises measuring an expression level of at least 2, at least 3, at least 4, at least 5, at least 6, at least 7, at least 8, at least 9, at least 10, at least 11, at least 12, at least 13, at least 14, at least 15, at least 16, at least 17, or each of Hpgd, Anapc 16, Sympk, Mblac2, Sepw1, Vash2, Ermard, Snhg7, Gm16845, Ppplr3fos, Metrn, Pik3c2b, Fam212a, Zfp560, Zfp747, Rnf10, Gnb4, and Git1. In some embodiments, the method comprises determining that the subject is at risk of or currently experiencing transplant rejection when the expression level of one or more of Hpgd, Anapc16, Sympk, Mblac2, Sepw1, Vash2, Ermard, Snhg7, Gm16845, Ppp1r3fos, Metrn, Pik3c2b, Fam212a, Zfp560, Zfp747, Rnf10, Gnb4, and Git1 is increased compared to a reference level (e.g. a control level or a cutoff level). In some embodiments, the method comprises determining that the subject is at risk of or currently experiencing transplant rejection when the expression level of one or more of Hpgd, Anapc16, Sympk, Mblac2, Sepw1, Vash2, Ermard, Snhg7, Gm16845, Ppplr3fos, Metrn, Pik3c2b, Fam212a, Zfp560, Zfp747, Rnf10, Gnb4, and Git1 is decreased compared to a reference level (e.g. a control level or a cutoff level).

In some embodiments, the method comprises measuring an expression level of one or more genes selected from Tmem234, Sfxn5, Zfp963, Rcsd1, Vwf, and Gm19897. In some embodiments, the method comprises measuring an expression level of at least 2, at least 3, at least 4, at least 5, or each of Tmem234, Sfxn5, Zfp963, Rcsd1, Vwf, and Gm19897. In some embodiments, the method comprises determining that the subject is at risk of or currently experiencing transplant rejection when the expression level of one or more of Tmem234, Sfxn5, Zfp963, Rcsd1, Vwf, and Gm 19897 is increased compared to a reference level (e.g. a control level or a cutoff level). In some embodiments, the method comprises determining that the subject is at risk of or currently experiencing transplant rejection when the expression level of one or more of Tmem234, Sfxn5, Zfp963, Rcsd1, Vwf, and Gm 19897 is decreased compared to a reference level (e.g. a control level or a cutoff level). In some embodiments, the method comprises determining that the subject is at risk of or currently experiencing transplant rejection when the expression level of one or more of Tmem234, Sfxn5, Zfp963, and Rcsdl is increased compared to a reference level. In some embodiments, the method comprises determining that the subject is at risk of or currently experiencing transplant rejection when the expression level of one or more of Vwf and Gm 19897 is decreased compared to a reference level. In some embodiments, the method comprises determining that the subject is at risk of or currently experiencing transplant rejection when the expression level of one or more of Tmem234, Sfxn5, Zfp963, and Rcsdl is increased compared to a reference level and the expression level of one or more of Vwf and Gm 19897 is decreased compared to a reference level. In some embodiments, the method comprises determining that the subject is at risk of or currently experiencing transplant rejection when the expression level of Tmem234, Sfxn5, Zfp963, and Rcsdl is increased compared to a reference level and the expression level of Vwf and Gm19897 is decreased compared to a reference level. In some embodiments, the transplant is a skin transplant. In some embodiments, the expression level of the one or more genes is measured within 14 days of the subject receiving the skin transplant.

In some embodiments, the method comprises measuring an expression level of one or more genes selected Tmem267, Sacd1, Cnp, Klhdc2, Igtp, Atpaf2, Arntl, Cds1, Nt5m, Ldhb, Plekhg3, Gbp3, Zbp1, Trnaulap, Rxra, Urod, and Fgl2. In some embodiments, the method comprises measuring an expression level of at least 2, at least 3, at least 4, at least 5, at least 6, at least 7, at least 8, at least 9, at least 10, at least 11, at least 12, at least 13, at least 14, at least 15, at least 16, or each of Tmem267, Sacd1, Cnp, Klhdc2, Igtp, Atpaf2, Arntl, Cds1, Nt5m, Ldhb, Plekhg3, Gbp3, Zbp1, Trnaulap, Rxra, Urod, and Fgl2. In some embodiments, the method comprises determining that the subject is at risk of or currently experiencing transplant rejection when the expression level of one or more of Tmem267, Sacd1, Cnp, Klhdc2, Igtp, Atpaf2, Arntl, Cds1, Nt5m, Ldhb, Plekhg3, Gbp3, Zbp1, Trnaulap, Rxra, Urod, and Fgl2 is increased compared to a reference level (e.g. a control level or a cutoff level). In some embodiments, the method comprises determining that the subject is at risk of or currently experiencing transplant rejection when the expression level of one or more of Tmem267, Sacd1, Cnp, Klhdc2, Igtp, Atpaf2, Arntl, Cds1, Nt5m, Ldhb, Plekhg3, Gbp3, Zbp1, Traulap, Rxra, Urod, and Fg12 is decreased compared to a reference level (e.g. a control level or a cutoff level). In some embodiments, the method comprises determining that the subject is experiencing or at risk of experiencing transplant rejection when the expression level of one or more of Cds1, Ntrm, Ldhb, Rxra, Urod, and Plekhg3 are decreased relative to a threshold value. In some embodiments, the method comprises determining that the subject is experiencing or at risk of experiencing transplant rejection when the expression level of each of Cds1, Nt5m, Ldhb, Rxra, Urod, and Plekhg3 is decreased relative to a threshold value. In some embodiments, the method comprises determining that the subject is in the pre-symptomatic stage of transplant rejection when the expression level of one or more of Rxra, Urod, and Plekhg3 is decreased relative to a threshold value. In some embodiments, the method comprises determining that the subject is experiencing or at risk of experiencing transplant rejection when the expression level of Atpaf2 and Arntl are increased relative to a threshold value. In some embodiments, the method comprises determining that the subject is in the pre-symptomatic stage of transplant rejection when the expression level of Atpaf2 and Arntl are increased relative to a threshold value. In some embodiments, the method comprises determining that the subject is experiencing or at risk of experiencing transplant rejection when the expression level of one or more of Cds1, Ntrm, Ldhb, Rxra, Urod, and Plekhg3 are decreased relative to a threshold value and the expression level of Atpaf2 and Arntl are increased relative to a threshold value. In some embodiments, the method comprises determining that the subject is in mid-symptomatic transplant rejection when the expression level of one or more of Tmem267, Sacd1, Cnp, Klhdc2, Igtp, Gbp3, Zbp1, Trnaulap, and Fgl2 is increased relative to a threshold value.

In some embodiments, the method comprises measuring an expression level of one or more genes selected from Padi2, Ufp3a, Zfp182, Arhgap0, Nmi, Prcc, Cbx7, Flywch1, Prcp, Slc30a7, Pura, H2-Eb1, and Klf9. In some embodiments, the method comprises measuring an expression level of at least 2, at least 3. at least 4, at least 5, at least 6, at least 7, at least 8, at least 9, at least 10, at least 11, at least 12, each of Padi2, Ufp3a, Zfp182, Arhgap0, Nmi, Prcc, Cbx7, Flywch1, Prcp, Slc30a7, Pura, H2-Eb1, and Klf9. In some embodiments, the method comprises determining that the subject is at risk of or currently experiencing transplant rejection when the expression level of one or more of Padi2, Ufp3a, Zfp182, Arhgap0, Nmi, Prcc, Cbx7, Flywch1, Prcp, Slc30a7, Pura, H2-Eb1, and Klf9 is increased compared to a reference level (e.g. a control level or a cutoff level). In some embodiments, the method comprises determining that the subject is at risk of or currently experiencing transplant rejection when the expression level of one or more of Padi2, Ufp3a, Zfp182, Arhgap0, Nmi, Prcc, Cbx7, Flywch1, Prcp, Slc30a7, Pura, H2-Eb1, and Klf9 is decreased compared to a reference level (e.g. a control level or a cutoff level).

Any suitable method may be used to determine expression levels. Suitable methods of determining expression levels of nucleic acids (e.g., mRNA) are known in the art and include amplification based techniques, such as quantitative polymerase chain reaction (qPCR), quantitative real-time PCR (qRT-PCR), and isothermal amplification methods (e.g. nicking endonuclease amplification reaction, transcription mediated amplification, loop-mediated isothermal amplification, helicase-dependent amplification, strand displacement amplification). Additional suitable methods for determining expression levels of nucleic acids include CRISPR-based detection methods, sequencing methods (e.g. Sanger sequencing, RNA sequencing, pyrosequencing, ion torrent sequencing, sequencing-by-synthesis, droplet-digital sequencing, sequencing-by-synthesis, etc.), Northern blotting and Southern blotting. Suitable sequencing technologies are reviewed in, for example, Zhong et al., Ann Lab Med. 2021 January; 41(1): 25-43, and Slatko et al., Curr Protoc Mol Biol. 2018 April; 122(1): e59., the entire contents of each of which are incorporated herein by reference.

Techniques for measuring gene expression include, for example, gene expression assays with or without the use of gene chips, which are described in Onken et al., J Molec Diag 12(4): 461-468 (2010); and Kirby et al., Adv Clin Chem 44:247-292 (2007). Affymetrix gene chips and RNA chips and gene expression assay kits (e.g., Applied Biosystems™ TaqMan® Gene Expression Assays) are also commercially available from companies, such as ThermoFisher Scientific (Waltham, Mass.).

Suitable methods of determining expression levels of proteins are known in the art and include immunoassays (e.g., Western blotting, an enzyme-linked immunosorbent assay (ELISA), a radioimmunoassay (RIA), and immunohistochemical assay) or bead-based multiplex assays, e.g., those described in Djoba Siawaya J F, Roberts T, Babb C, Black G, Golakai H J, Stanley K, et al. (2008) An Evaluation of Commercial Fluorescent Bead-Based Luminex Cytokine Assays. PLOS ONE 3(7): e2535. Additional exemplary methods for determining expression levels of protein include proteomic analysis, or the systematic identification and quantification of proteins of a particular biological system. Mass spectrometry, protein chips, and protein microarrays may be used for proteomic analysis.

In some embodiments, transplant status is monitored in the subject by analyzing cells obtained from the sample. For example, cell type analysis may be performed on the sample obtained from the subject following transplant, and compared to cell types present in a control sample (e.g. a sample obtained from the subject prior to transplant, or a sample obtained from a control subject). In some embodiments, the cell types present in the sample obtained following transplant may be assessed to determine whether the subject is currently experiencing or at risk of transplant rejection. For example, enhanced amounts of cell types that secrete pro-inflammatory cytokines and/or chemokines, antigen-presenting cell types, costimulatory cell types, and the like (e.g. B-cells, T-cells, (e.g. CD4+ cells, CD8+ cells) macrophages, granulocytes (e.g. neutrophils, eosinophils, basophils), natural killer cells, dendritic cells, etc.) may indicate that the subject is at risk of or currently experiencing transplant rejection.

In some embodiments, transplant status is monitored in the subject by analyzing cells obtained from the sample at the first time point and analyzing cells obtained from the sample at the second time point For example, cell type analysis may be performed at the first and second time points, and the subpopulations of cells present at the second time point compared to the first time point may be used to determine whether the subject is currently experiencing or at risk of transplant rejection For example, enhanced amounts of cell types that secrete pro-inflammatory cytokines and/or chemokines, antigen-presenting cell types, costimulatory cell types, and the like (e.g B-cells, T-cells, (e.g CD4+ cells, CD8+ cells) macrophages, granulocytes (e.g. neutrophils, eosinophils, basophils), natural killer cells, dendritic cells, etc.) at the second time point compared to the first time point may indicate that the subject is at risk of or currently experiencing transplant rejection.

Various methods for analyzing cells in the sample may be used. In some embodiments, methods for analyzing cells may comprise separating cells into cell type specific sub-populations. In some embodiments, the sample containing the cells of interest may be contacted with one or more labels (e.g. antibodies, each antibody comprising a fluorescent molecule) and the label can be used to sort and/or identify cell types of interest. For example, cells may be separated into cell types by known techniques, including, for example, flow cytometry, fluorescent-assisted cell sorting (FACS), magnetic-assisted cell sorting, histological techniques, e.g., fluorescent immunohistochemistry, or multiplexed fluorescent imaging technologies. In exemplary aspects, the methods comprise monitoring the cell populations over time. In exemplary aspects, the methods comprise measuring or quantifying the different cell populations in the sample, in addition to or instead of measuring a level of expression of a gene, an RNA or a protein, in the sample.

In some embodiments, the methods further comprise providing a treatment to the subject. For example, in some embodiments the methods comprise treating the subject with a suitable treatment regimen based upon the transplant status in the subject For example, the method may comprise monitoring the subject if the subject is not determined to be at risk of or currently experiencing transplant rejection If the subject has already received a first anti-rejection therapy, the method may comprise reducing the dose of the first anti-rejection therapy or ceasing treatment with the first anti-rejection therapy if the subject is not determined to be at risk of or currently experiencing transplant rejection. As another example, the method may comprise providing an anti-rejection therapy to the subject if the subject is determined to be at risk of or currently experiencing transplant rejection. If the subject has already received a first anti-rejection therapy, the method may comprise increasing the dose of the first anti-rejection therapy and/or providing a different anti-rejection therapy to the subject if the subject is determined to be at risk of or currently experiencing transplant rejection.

Suitable anti-rejection therapies include, for example, immunosuppressive agents Suitable immunosuppressive agents include, for example, corticosteroids (e.g. prednisolone, hydrocortisone), calcineurin inhibitors (e.g. Ciclosporin, Tacrolimus), anti-proliferative agents (e.g. Azathioprine, Mycophenolic acid), mTOR inhibitors (e.g. Sirolimus, Everolimus), and the like. Additional suitable anti-rejection therapies include antibody-based treatments, including monoclonal antibodies such as anti-IL-2Rα antibodies (e.g. Basiliximab, Daclizumab), anti-IL-6R antibodies (Tocilizumab), anti-CD20 antibodies (Rituximab), and polyclonal antibodies such as polyclonal anti-T-cell antibodies (e.g anti-thymocyte globulin, anti-lymphocyte globulin), and the like. Any one or more anti-rejection therapies may be provided to the subject. In some embodiments, the subject has already received an immunosuppressive agent, and the methods described herein may be used to monitor transplant rejection in the subject to determine whether the immunosuppressive agent is effective at treating and/or preventing transplant rejection in the subject.

In some embodiments, the methods described herein may be used to monitor the efficacy of anti-rejection treatment(s) provided to the subject. For example, the methods may be performed to monitor treatment status in the subject. Monitoring treatment status may comprise monitoring whether the treatment provided to the subject (e.g immunosuppressive therapy, antibody-based therapy) effectively treats transplant rejection in the subject. Successful treatment of transplant rejection may be determined if the expression levels of the one or more genes, RNA, and/or proteins in the subject are substantially returned to a control level (e.g. a baseline level), or cutoff level in the subject. For example, expression levels of one or more genes, RNA, and/or protein may be increased in the subject following transplant, thus indicating that the subject is at risk of or currently experiencing transplant rejection. As another example, successful treatment of transplant rejection may be determined if subtypes of cells present in a sample, and/or relative amounts of various cell types in a sample obtained following transplant are substantially similar to that of a control sample. One or more suitable anti-rejection therapies may be provided to the subject (e.g. immunosuppressive therapy, antibody-based therapy, or a combination thereof), and the expression levels of the one or more genes, RNA, and/or protein may be measured following therapy. A substantial return of the expression level to a desired level (e.g. to a control level, or to a baseline level obtained from the subject prior to transplant) indicates successful treatment of transplant rejection in the subject.

In some aspects, provided herein are kits. In some embodiments, provided herein is a kit comprising a synthetic scaffold described herein. The kit may comprise additional components, such as additional materials required for implanting the scaffold in the subject, collecting the scaffold or a portion thereof from the subject (e.g. tweezers, microneedles, tubes, etc.), and analyzing expression of one or more genes, nucleic acids, or proteins in the sample. For example, the kit may additionally comprise components necessary for analysis of gene expression or analysis of RNA expression and/or analysis of protein expression in the sample (e.g primers, probes, antibodies, buffers, inhibitors, stabilizers, salts, denaturants, and other suitable reagents). In some embodiments, the kit comprises instructions for use.

### Example 1

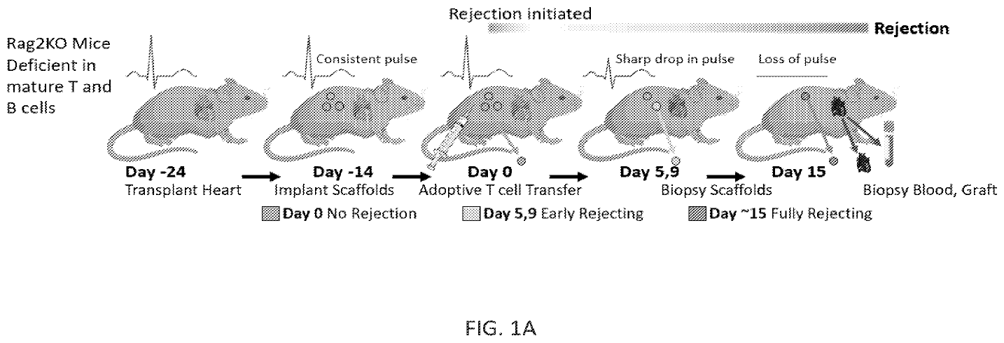

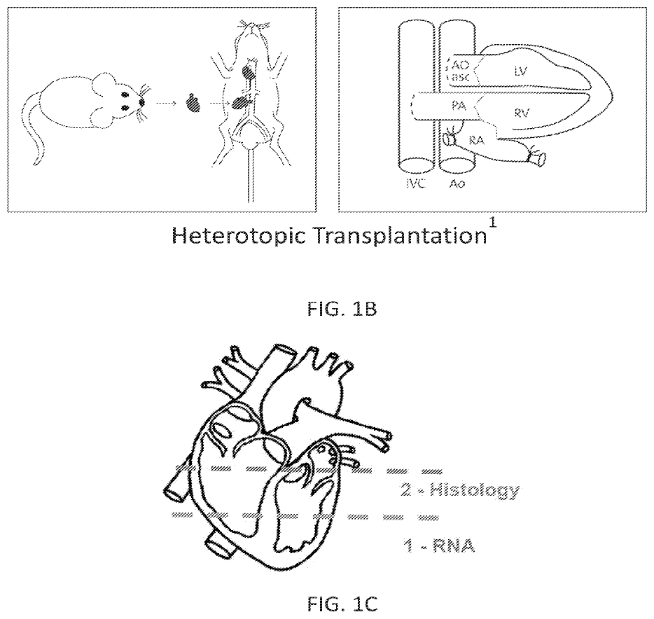

Rag2 knockout (RAG2KO) mice deficient in mature T and B cells were used to measure gene expression in an animal model of heart transplant rejection. FIG. 1A is a schematic showing an exemplary protocol for a heart transplant and subsequent monitoring using the materials and methods described herein. Heterotopic heart transplant was performed at day −24. Synthetic scaffolds were implanted at day −14. Adoptive transfer of T cells began at day 0. T cells were obtained from the spleen of sex and age matched C57B16 mice.

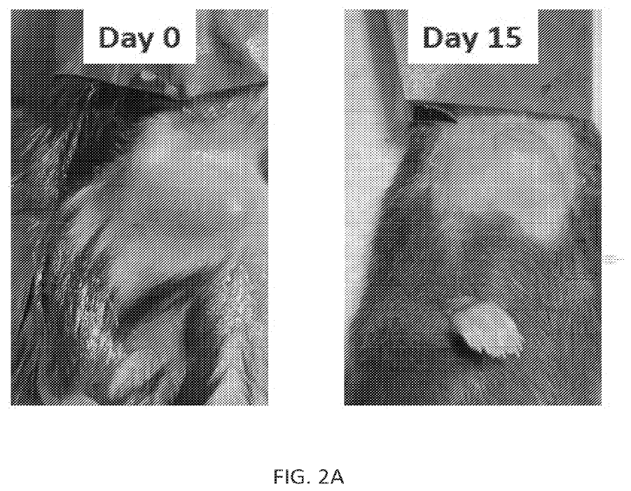

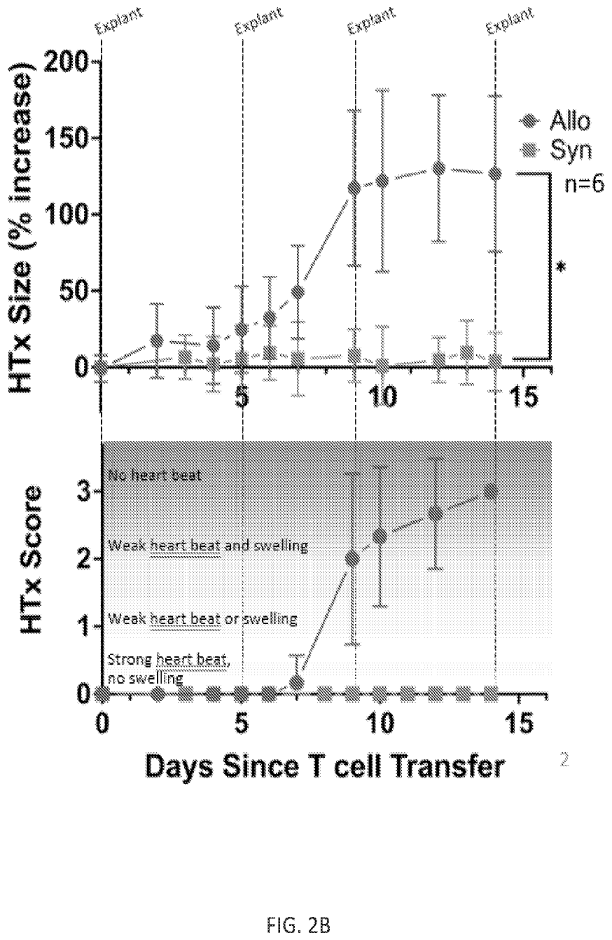

The synthetic scaffolds were biopsied at Days 0, 5, 9, and 15 for analysis by RNAseq and histology. FIGS. 2A-2B show measurements of transplanted heart function. FIG. 2A is an image at day 0 and day 15 following transplant. FIG. 2B shows heart transplant size (top graph) and score based upon strength of heartbeat and swelling (bottom graph).

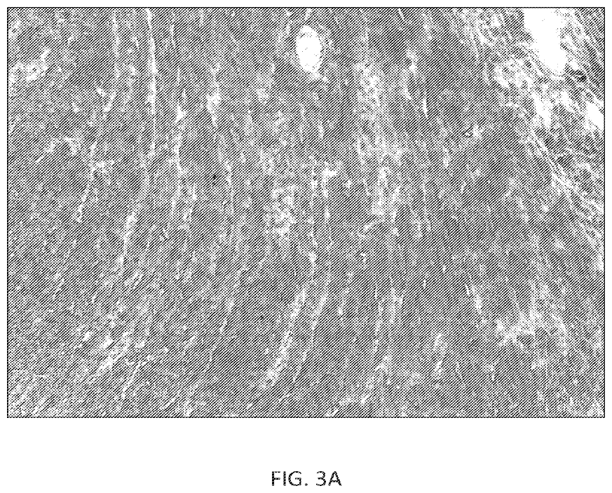

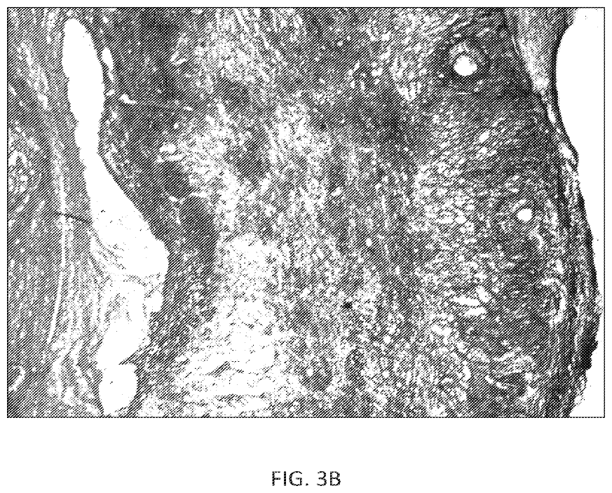

FIGS. 3A-3B show a comparison of syngeneic and allogeneic heart transplants. FIG. 3A shows histology (H&E staining) from a syngeneic heart graft at day 15 post adoptive T cell transfer. The transplant received a rejection score of 0, as there was no swelling observed and a strong heartbeat. FIG. 3B shows a histology (H&E staining) from an allogeneic heart graft at day 15 post adoptive T cell transfer. The transplant received a rejection score of 3, as it was very swollen and there was no heartbeat. Images are representative from n=3 animals per group, with all microscope settings identical.

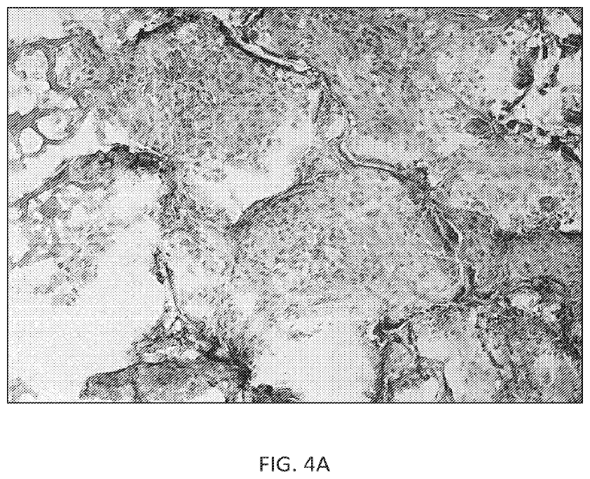

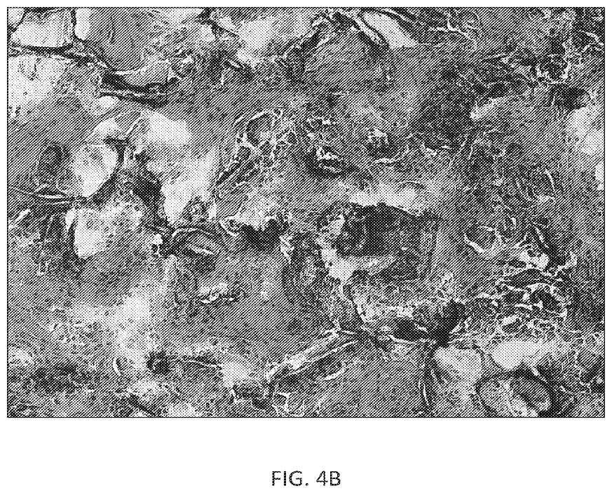

FIGS. 4A-4B show a comparison of scaffolds obtained from animals with syngeneic and allogeneic heart transplants. FIG. 4A shows histology (H&E staining) from a scaffold from a syngeneic heart graft at day 9. The transplant received a rejection score of 0, as there was no swelling observed and a strong heartbeat. FIG. 4B shows a histology (H&E staining) from a scaffold from an allogeneic heart graft at day 9. The transplant received a rejection score of 3, as it was very swollen and there was no heartbeat. Images are representative from n=3 animals per group, with all microscope settings identical.

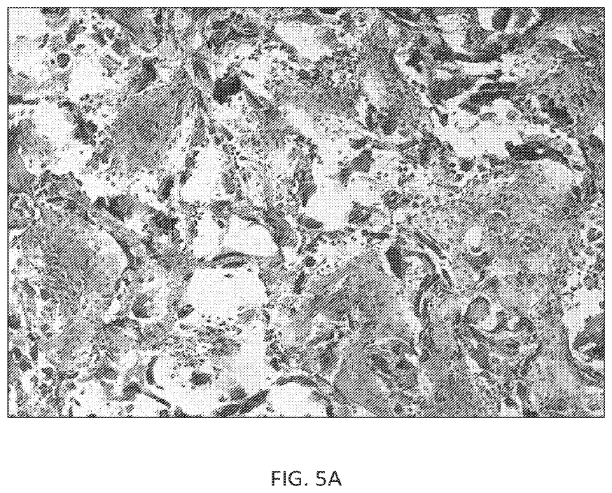

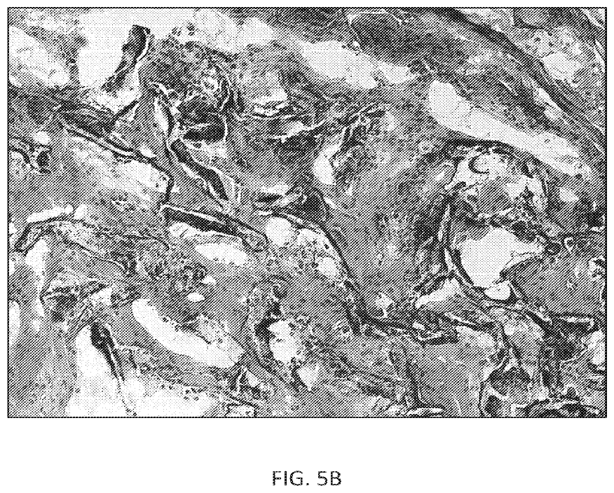

FIGS. 5A-5B show a comparison of scaffolds obtained from animals with syngeneic and allogeneic heart transplants. FIG. 5A shows histology (H&E staining) from a scaffold from a syngeneic heart graft at day 15. The transplant received a rejection score of 0, as there was no swelling observed and a strong heartbeat. FIG. 5B shows a histology (H&E staining) from a scaffold from an allogeneic heart graft at day 15. The transplant received a rejection score of 3, as it was very swollen and there was no heartbeat. Images are representative from n=3 animals per group, with all microscope settings identical.

Taken together, these results demonstrate that the implanted scaffolds were able to provide tissue indicative of transplant rejection status.

### Example 2

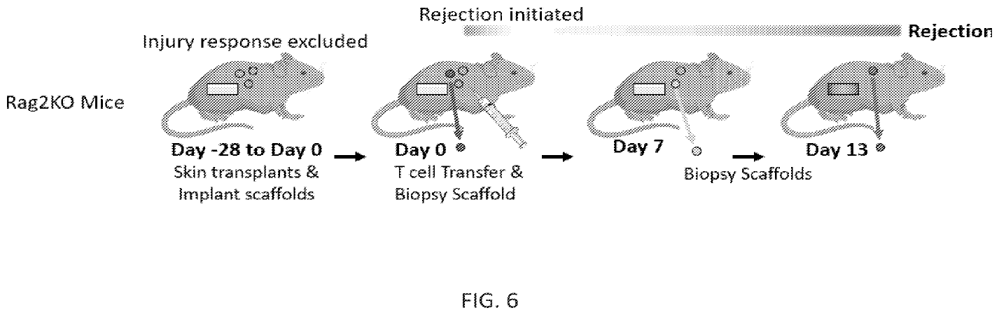

Rag2 knockout (RAG2KO) mice deficient in mature T and B cells were used to measure gene expression in an animal model of skin transplant rejection. FIG. 6 is a schematic showing an exemplary protocol for using the materials disclosed herein for monitoring skin graft rejection. From day −28 to day 0 skin transplants were performed and scaffolds were implanted. At day 0, T cells were transferred. T cells were obtained from the spleen of sex and age matched C57B16 mice. Scaffolds were biopsied at day 0, day 7, and day 13.

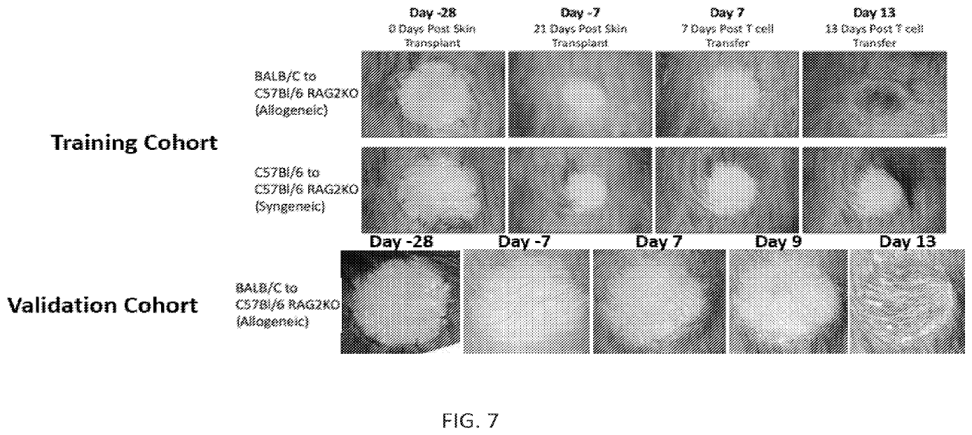

FIG. 7 shows images of the skin graft site in training and validation cohorts at various time points. Images are representative images of n=3 animals per group.

FIG. 8 shows analysis of RNAseq data to obtain a skin rejection scaffold signature of rejection associated transcripts (RATs). RNAseq data was analyzed for the multiple samples. Raw counts were normalized by DESeq2. Each gene underwent hypothesis testing comparing rejecting transplants to healthy transplants to identify a subset of differentially expressed genes. This subset of genes was then analyzed for greatest positive or negative fold change and false discovery rate to determine a panel of fewer than 30 genes that distinguish between scaffolds from mice with rejecting and non-rejecting transplants. A volcano plot of 14 genes identified is shown in FIG. 8A. A heatmap of z-scores for the 14 genes is shown in FIG. 8B.

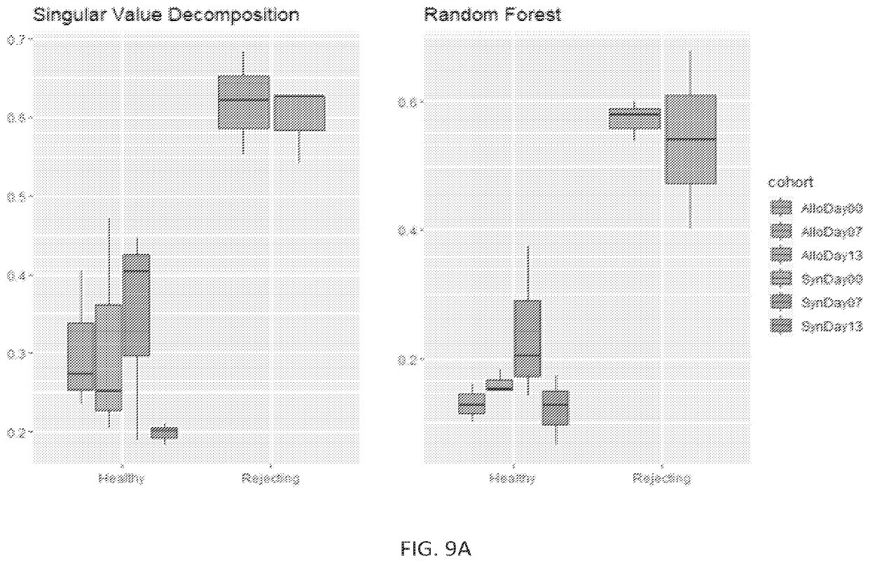

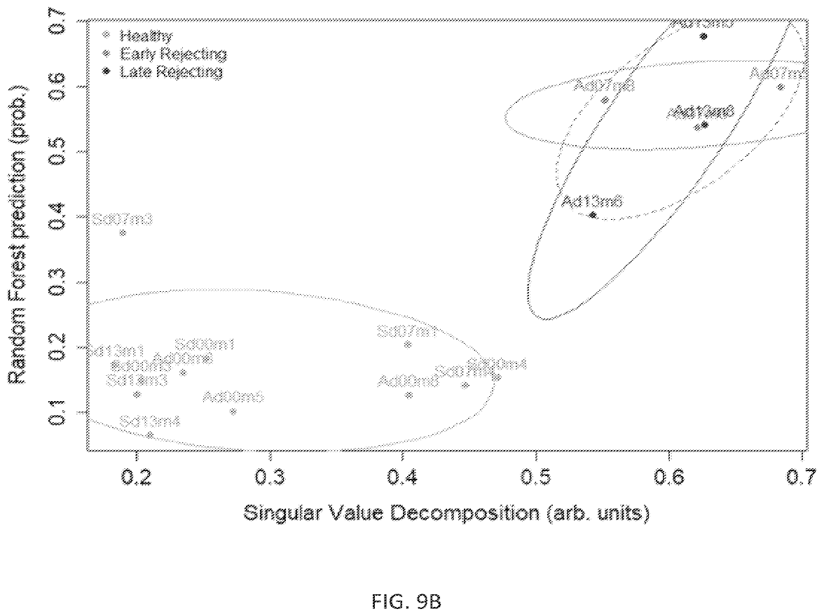

FIG. 9A-9B show a two metric scoring system based upon differential gene expression. FIG. 9A shows singular value decomposition SVD (supervised) and Random Forest (unsupervised). The gene expression can then be converted to two numbers that reflect a position in the graph of FIG. 9B. Scaffolds from healthy or non-rejecting mice are in blue, and generally had a low score by both SVD and random forest. Scaffolds from early or late rejecting transplanted mice had a high score in both scoring systems.

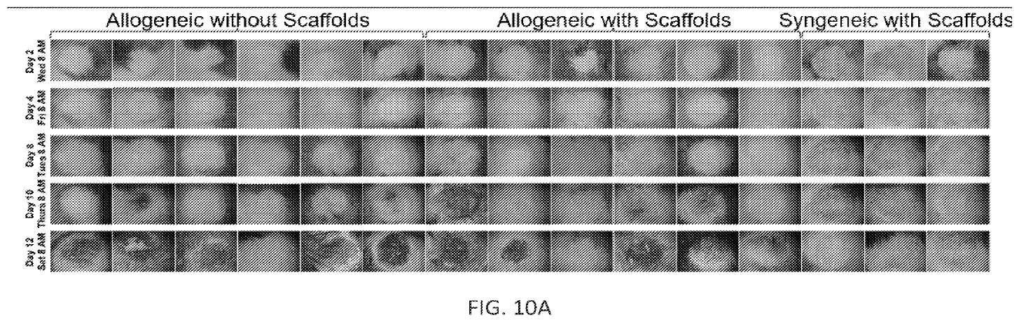

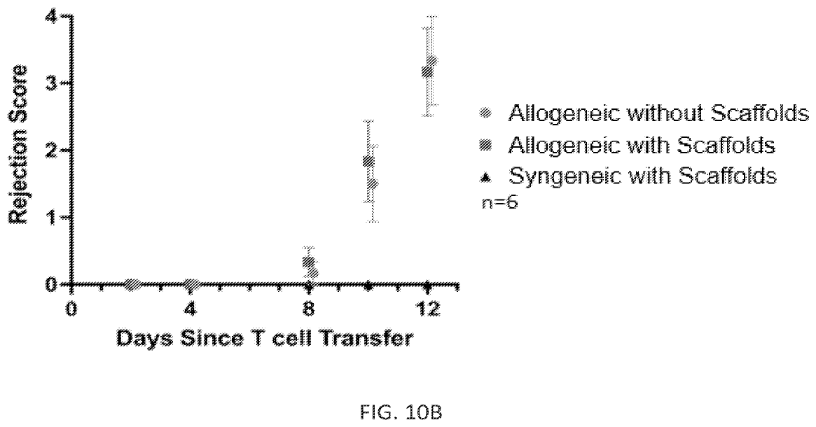

FIG. 10A-10B demonstrate that scaffolds do not affect rejection following a skin graft. FIG. 10A shows images from allogeneic skin grafts with and without scaffolds, and from syngeneic skin grafts with scaffolds. FIG. 10B quantifies rejection score for all groups. Syngeneic graft do not reject, regardless of the presence of scaffolds.

Taken together, the results demonstrate that samples obtained from the synthetic scaffolds implanted in the mice can be used and gene expression can be measured to determine which animals are experiencing rejection. In this example, upregulation of X1700008J07Rik, Bmp4, Spc24, Rasgrp1, Trmt12, Aimp2, Lrch3, Ube2e2, X2210011C24Rik, Abeb8, Ces2g. Isg20, X950091008Rik, and Acot2 was observed and downregulation of Aimp2, Lrch3, and Trmt 12 was observed in samples obtained from animals experiencing rejection.

### Example 3

In this example, microporous scaffold implants are demonstrated to accumulate immune cells, producing biomarkers of acute cellular allograft rejection (ACAR) as an engineered immunological niche. Poly-caprolactone scaffolds were subcutaneously implanted in murine heart or skin transplant recipients to remotely identify ACAR before evidence of graft injury occurred. RAG2″-mice received full MHC mismatch allografts, and ACAR was initiated as a tunable event by the adoptive transfer of syngeneic C57BL/6 T cells. Scaffolds were explanted at various stages of rejection and sequenced for differential gene expression and pathway enrichment. Novel gene biomarker panels that distinguish mice with rejecting grafts from those with healthy grafts in a tissue-specific or tissue-independent context were identified. This gene signature identifies ACAR distinct from respiratory infection, without invasive graft biopsy before symptom onset. This implantable scaffold enables remote evaluation of the early risk of rejection to reduce the frequency of routine graft biopsy and personalize immunosuppression that could prolong transplant life while minimizing patient risk.

### RESULTS

### Scaffold Implants do not Accelerate Acute Rejection Tempo

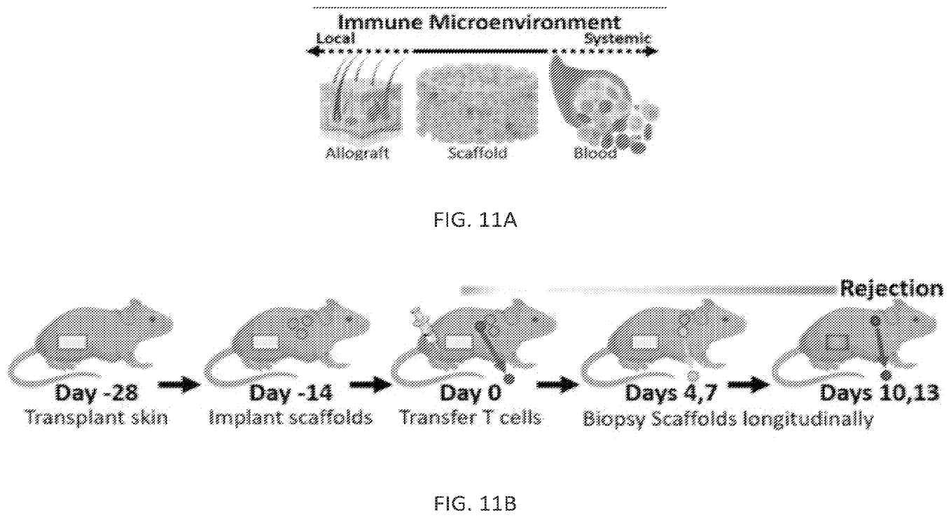

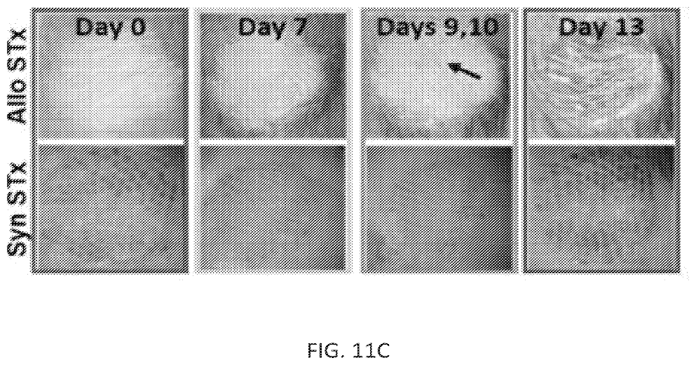

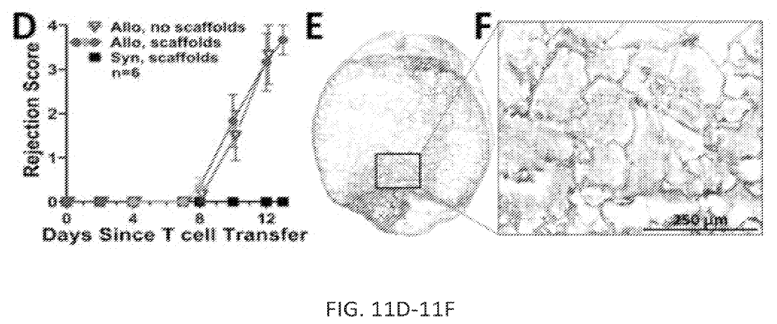

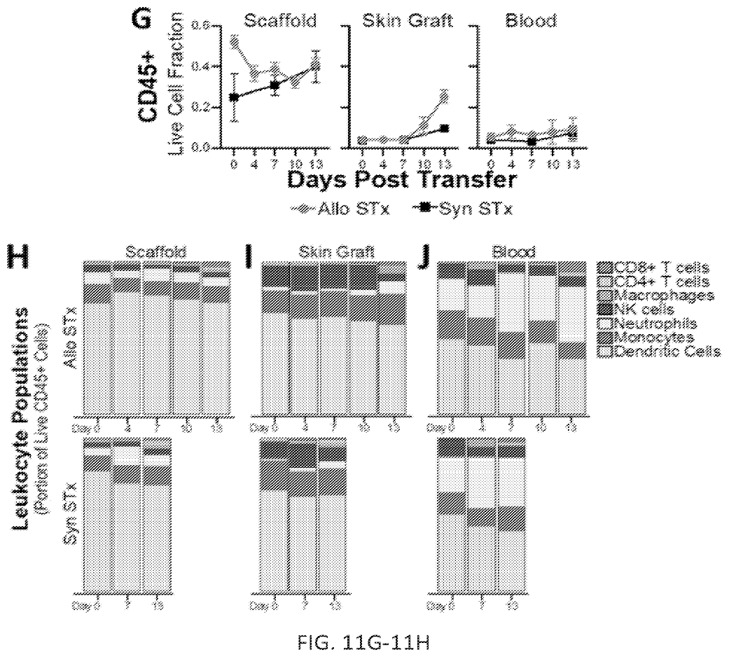

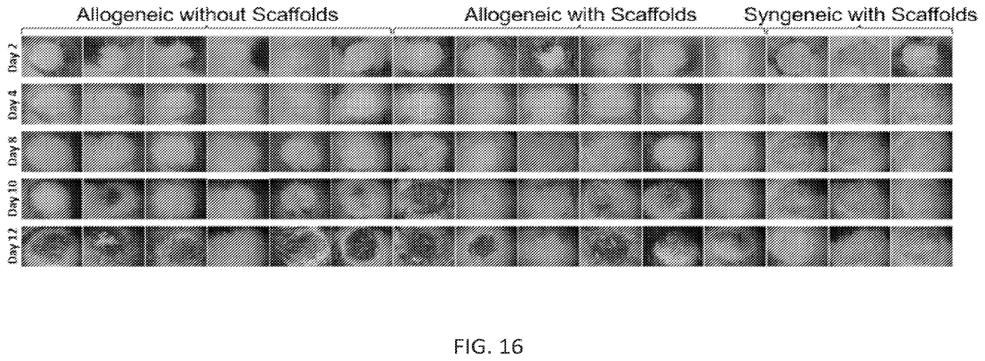

When implanted subcutaneously, poly-caprolactone (PCL) scaffolds become vascularized and promote immune cell infiltration to continuously sample both innate and adaptive responses. These immune dynamics occur with a tissue-like phenotype compared to the blood, bridging the local and systemic immune responses of ACAR (FIG. 11A). Cell capture scaffolds were generated to collect predictive biomarkers in a murine skin graft model of tunable ACAR. Full major histo-compatibility mismatch skin transplants (STx) were performed (FIG. 11B) by grafting tail skin onto the dorsum of RAG2^{-/-} immunodeficient mice in allografts (B/c onto RAG2^{-/-}) or syngeneic grafts (B6 onto RAG2^{-/-}). Grafts healed over 24 days, and scaffolds were implanted subcutaneously 14 days prior to initiating ACAR. Allorecognition was initiated by adoptively transferring 10⁷ T cells from naïve B6 donors to stratify graft recipients into those rejecting allografts or those with healthy syngeneic grafts (FIGS. 11C, D; FIG. 16). ACAR then developed over approximately 13 days, with symptoms (i.e., wound formation) visible beginning 9 or 10 days following T cell transfer and complete graft wounding on day 13 (FIGS. 11C, D). As such, day 7 was considered a pre-symptomatic timepoint for ACAR. Implanted subcutaneous scaffolds and their longitudinal explants after adoptive T cell transfer did not alter the tempo of ACAR (FIG. 11D). When syngeneic T cells were adoptively transferred on day 0, two weeks after scaffold implant, scaffolds were well vascularized and contained widespread cell intravasation (FIGS. 11E, F) and were thus suitable for explant and subsequent sequencing and analyses.

### Implanted Scaffolds Capture Predominantly Innate Immune Cells

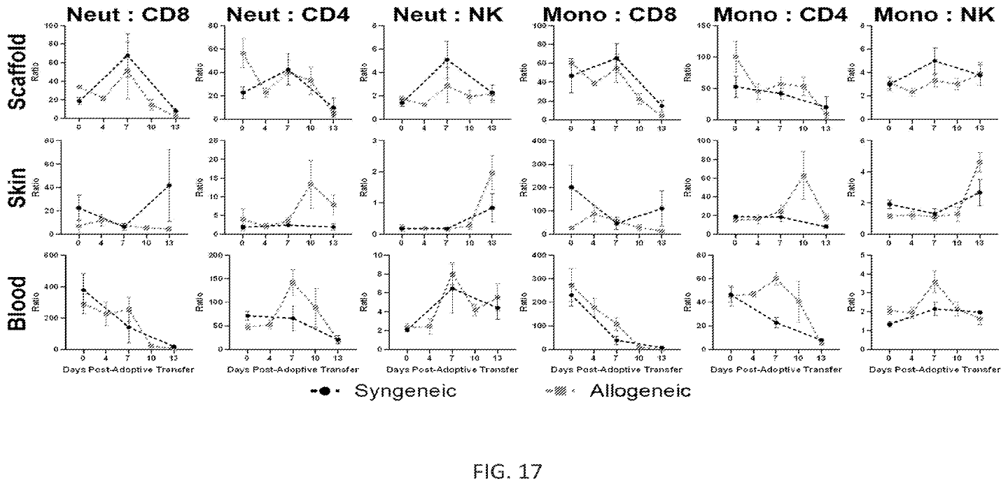

At day 0 prior to T cell transfer and at stages of pre-symptomatic and symptomatic ACAR, single cell suspensions from scaffold explants, skin grafts, and cardiac blood draws were generated and assessed for leukocyte populations via flow cytometry (FIGS. 11G-J; FIG. 17). Scaffolds captured an approximately three-fold greater proportion and number of leukocytes than identified in circulation or in the graft itself before symptoms of graft injury (FIG. 11G). Also, allogeneic skin grafts show an influx of CD45″ cells corresponding with ACAR symptom onset (FIG. 11G) which is absent in syngeneic grafts that do not undergo ACAR. This further identifies day 7 and earlier as pre-symptomatic timepoints of ACAR at which rejection would be poorly identified through histological analysis, assays of T cell proliferation, or graft injury.

Scaffolds captured predominantly innate immune cells (FIG. 11H), with a greater proportion of DCs than found at the skin graft or in the blood (FIGS. 11I,J). As allograft rejection is largely mediated by antigen presenting cells, the high proportion of DCs may be favorable for identifying early allorecognition through the scaffold. Neutrophil proportion, by contrast, is diminished at the scaffold (FIG. 11H) in comparison to the blood (FIG. 11J). Leukocyte proportions changed little, however, within each tissue during ACAR progression or compared to syngeneic graft recipients (FIGS. 11H-J), until the graft was infiltrated by CD4⁺ and CD8⁺ T cells on day 13, corresponding to late ACAR and wounding (FIG. 11I). This T cell expansion was also found in circulation and at the scaffold (FIGS. 11H-J). These conserved leukocyte proportions and ratios (FIG. 17) prior to symptomatic ACAR emphasize the need to assess the specific phenotype and gene expression profile of these immune cells. This also highlights the limitations of graft biopsy and gene expression profiling which assess the T cell proliferation dynamics of symptomatic ACAR.

### Pathways Enriched in the Scaffold Recapitulate ACAR Dynamics

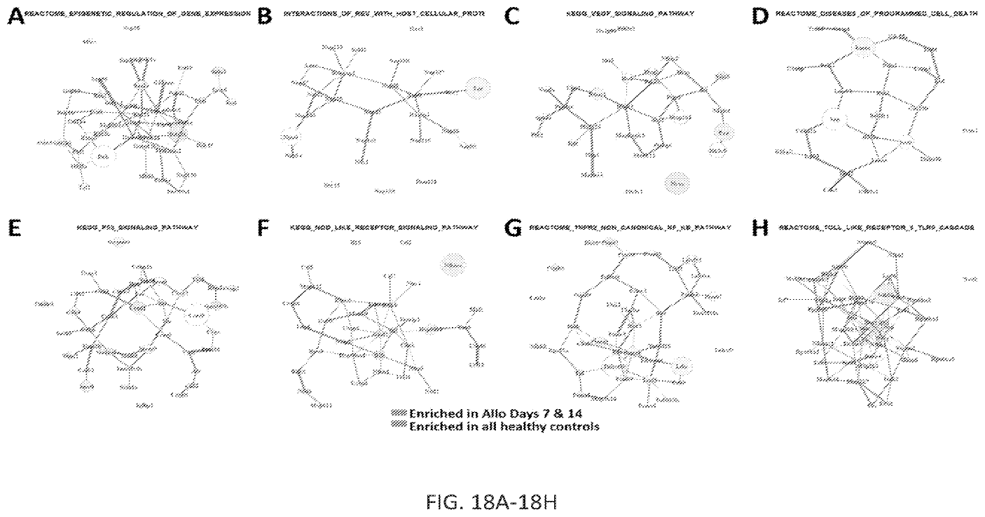

To assess gene expression at the scaffold and identify predictive biomarkers of ACAR, RNA was extracted from longitudinal scaffold explants in an additional STx cohort and sequenced (RNA-seq). Gene expression was then filtered to remove unexpressed, lowly expressed, and uniformly expressed genes and analyzed for differential enrichment and expression between healthy graft recipients and recipients undergoing pre-symptomatic or symptomatic ACAR at both the pathway and gene levels (FIG. 12A). Gene set enrichment analysis (GSEA) identified differentially-enriched pathways at the scaffold with a low false discovery rate between healthy recipients and either pre-symptomatic, late symptomatic, or both ACAR groups (FIG. 12B; Table 1). Overall, pathway enrichment at the scaffold in pre-symptomatic ACAR is more dissimilar to healthy recipients than those in late symptomatic ACAR (FIG. 12B second column), suggesting that at the late stage of ACAR in which the graft has complete wounding the immunological rejection is resolving. Numerous differentially regulated T cell pathways associated with activation and differentiation were readily identified in pre-symptomatic ACAR (FIG. 12B third column), despite very few T cells present at the scaffold at Day 7 (FIG. 11H). Despite the enrichment of these T cell processes, the scaffold recapitulates predominantly myeloid cell-related activity during pre-symptomatic ACAR, while being further enriched for T cell-related pathways during late ACAR (FIG. 12B fourth column) reflective of systemic and graft-localized T cell dynamics. Notably, while the myeloid cell proportions remain largely unchanged as ACAR progresses and are indistinguishable from syngeneic graft recipients (FIGS. 11H-J), processes enriched and depleted at the scaffold during ACAR demonstrate the differential expression and activity of these myeloid cells. The differential regulation of these pathways was mapped as expression networks (FIG. 18). Notably, the Hallmark Database pathway of Allograft Rejection is enriched at the scaffold during pre-symptomatic ACAR (FIG. 12C), with upregulation of Ccl4, CD74, and Tnf, downregulation of Mmp9 and Rps9, and depleted relationships between Cdld1-1116, Cd28-Stat4, and CD80-Cxcr3-among others. Gene set variation analysis (GSVA) was applied to the RNA-seq data and then an Elastic Net regression performed to specify the fewest pathways needed to distinguish ACAR from healthy grafts (FIG. 19; Table 1). These differentiating pathways include processes associated with epigenetic regulation of cell death, apoptosis, and cytomegalovirus infection.

TABLE 1
Pathways and genes differentially represented at the scaffold during ACAR in STx.

| | | | STx Serial |
|---|---|---|---|
| STx Differentially Enriched Pathways | STx DEGs | STx Panel | Panel |
| FOXP3 Targets in Thymus Upregulated | Igfbp2 | Hpgd | Tmem234 |
| IRF4 Targets in Activated Dendritic Cell | Dnajb2 | Anapc16 | Sfxn5 |
| Mature B Lymphocyte Upregulated | Slc4a3 | Sympk | Zfp963 |
| Differentiating T Lymphocyte | Per2 | Mblac2 | Rcsd1 |
| Abnormal Lymphocyte Morphology | Ptpn4 | Sepw1 | Vwf |
| Bone Marrow Immature Neutrophil | Mfsd4 | Vash2 | Gm19897 |
| Hallmark IL6 JAK STAT3 Signaling | Aspm | Ermard | |
| Hallmark Allograft Rejection | Brinp3 | Snhg7 | |
| LPS vs LPS Low Monocyte Downregulated | Pcp4l1 | Gm16845 | |
| Unstim vs FCGRIIB Stim DC Upregulated | Ptprk | Ppp1r3fos | |
| Tconv PLN Downregulated vs CD24Hi Tconv Thymus | Fig4 | Metrn | |
| DC Upregulated vs Basophil | Cirbp | Pik3c2b | |
| Conventional vs Plasmacytoid DC Spleen Upregulated | Eid3 | Fam212a | |
| CD8 Neg DC Spleen vs CD8 Pos DC Spleen | Rpl41 | Zfp560 | |
| Upregulated | | | |
| Monocyte Upregulated vs MDC | Sap30l | Zfp747 | |
| Unstim vs LPS Stim Macrophage Down | Dhrs7b | Rnf10 | |
| Naive DC4 T cell vs Day 5 IL4 Conv Treg Down | Camkk1 | Gnb4 | |
| Wildtype vs MYD88KO Macrophage Down | Ccl2 | Git1 | |
| Naïve T cell vs Neutrophil Upregulated | Ska2 | | |
| Naïve CD4 T cell vs Neutrophil Upregulated | Hoxb5 | | |
| Naïve CD4 T cell vs Monocyte Down | Snord104 | | |
| Naïve vs 24 hr in vitro Stim CD8 T cell Down | Cep131 | | |
| Nonsuppressive T cell vs Activated Treg Upregulated | Acot2 | | |
| Naïve vs Memory CD8 T cell Upregulated | Ifi27l2a | | |
| Regulation of Inflammatory Response to Antigenic | Trmt6la | | |
| Stimulus | | | |
| Negative Regulation of MAP Kinase Activity | Gng4 | | |
| Myeloid Leukocyte Activation | Hist1h1e | | |
| Lymph Vessel Development | Gm16907 | | |
| Leukocyte Proliferation | Ttc37 | | |
| Granulocyte Activation | Nt5dc2 | | |
| Cell Activation involved in Immune Response | Bmp4 | | |
| Alpha Beta T cell Proliferation | Dlgap5 | | |
| Duodenal Immune Cells | Abhd4 | | |
| | Esco2 | | |
| | Cldn10 | | |
| | Ropn1l | | |
| | Tnfrsf11b | | |
| | Trmt12 | | |
| | Fam203a | | |
| | Tef | | |
| | Creld2 | | |
| | Kif21a | | |
| | Pus7l | | |
| | Kansl2 | | |
| | Itgb7 | | |
| | Mettl22 | | |
| | Lrch3 | | |
| | Zbtb20 | | |
| | Bace2 | | |
| | U2af1 | | |
| | Gabbr1 | | |
| | H2.M2 | | |
| | Ptk7 | | |
| | Dus3l | | |
| | Fam98a | | |
| | Arap3 | | |
| | Vps37c | | |
| | Slc25a28 | | |
| | Gata3 | | |
| | Thnsl1 | | |
| | Tubb4b | | |
| | Snapc4 | | |
| | Hspa5 | | |
| | Tnfaip6 | | |
| | Dusp19 | | |
| | Aplnr | | |
| | Foxs1 | | |
| | Gm14420 | | |
| | Slco4a1 | | |
| | Zbtb46 | | |
| | Med12l | | |
| | Mnd1 | | |
| | Cd5l | | |
| | Txnip | | |
| | Cd101 | | |
| | Alpk1 | | |
| | Gipc2 | | |
| | Bach2os | | |
| | Pnrc1 | | |
| | Nans | | |
| | Alad | | |
| | Pdzk1ip1 | | |
| | Btbd19 | | |
| | AU022252 | | |
| | Ppcs | | |
| | Pla2g2d | | |
| | Rps19.ps3 | | |
| | Abcb8 | | |
| | Fndc4 | | |
| | Tada2b | | |
| | Uchl1 | | |
| | Brdt | | |
| | Zfp605 | | |
| | Vsig10 | | |
| | Ephb4 | | |
| | Aimp2 | | |
| | Mtus2 | | |
| | Peg10 | | |
| | Ptn | | |
| | Zfp862.ps | | |
| | St3gal5 | | |
| | Mcm2 | | |
| | Gdf3 | | |
| | Gsg1 | | |
| | Klhl42 | | |
| | Hspbp1 | | |
| | Rps19 | | |
| | Tgfb1 | | |
| | Sertad3 | | |
| | Gm19897 | | |
| | Lin37 | | |
| | Cep89 | | |
| | Fah | | |
| | Coa4 | | |
| | Spon1 | | |
| | Rabep2 | | |
| | Map2k7 | | |
| | Efnb2 | | |
| | Snx25 | | |
| | Tll1 | | |
| | Npy1r | | |
| | Zfp961 | | |
| | Ces2g | | |
| | Tango6 | | |
| | Arhgap42 | | |
| | Slc36a4 | | |
| | Ppan | | |
| | Rnf26 | | |
| | Ttc12 | | |
| | Pih1d2 | | |
| | Rasgrf1 | | |
| | Rpgr | | |
| | Usp11 | | |
| | Zfp449 | | |
| | Prrg3 | | |
| | Tceanc | | |

### Scaffolds Identify a Biomarker Panel of Early ACAR in Skin Transplants

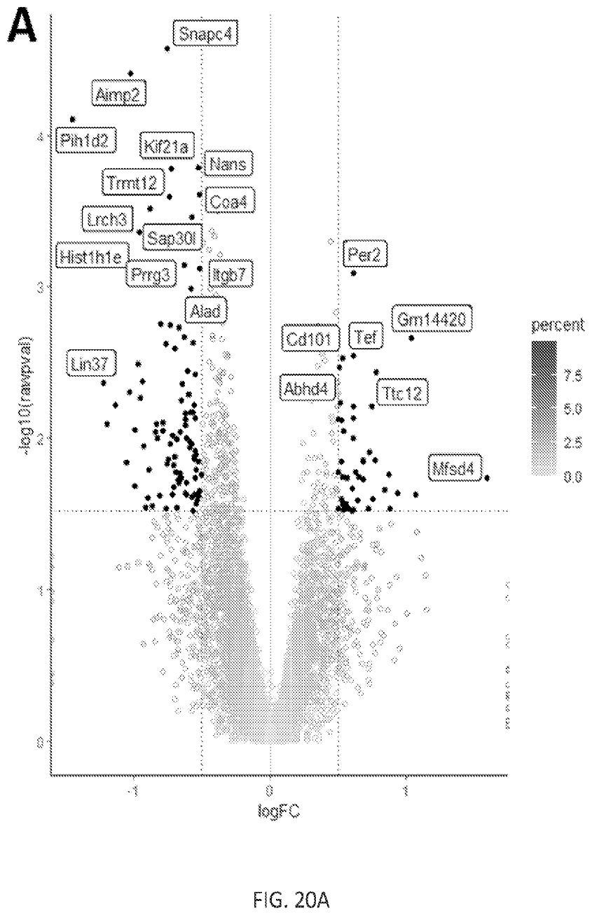

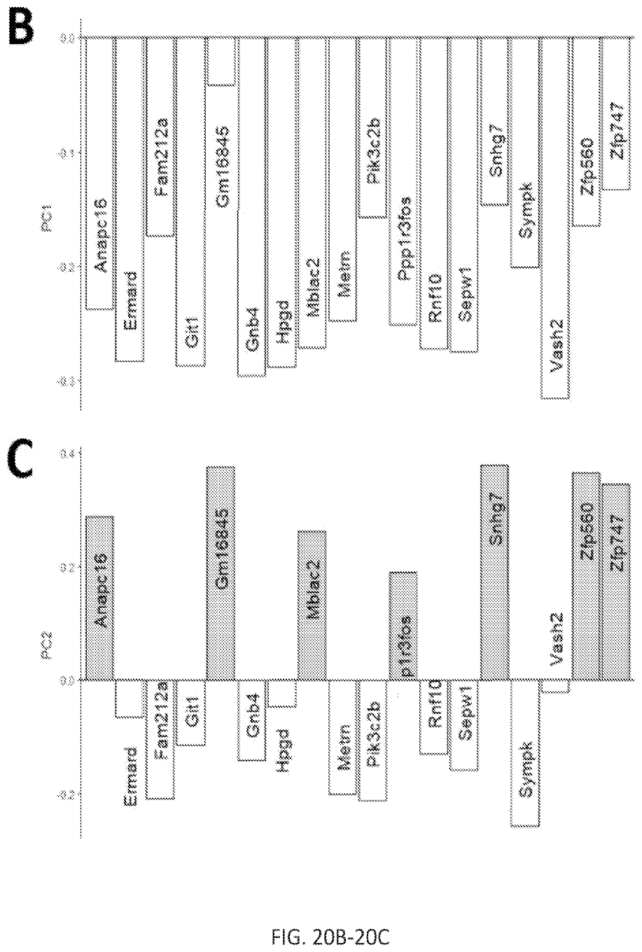

136 highly differentially expressed genes derived at the scaffold during ACAR were identified (FIG. 20). To create a sparse diagnostic biomarker panel of ACAR in STx with the fewest necessary indicators, a pathway-agnostic Elastic Net regression was developed, which identified 18 differentially expressed biomarkers (FIG. 12D; Table 1). 11 of these 18 genes are downregulated at the scaffold during pre-symptomatic ACAR and late ACAR compared to healthy graft recipient scaffolds, while 7 genes are downregulated in pre-symptomatic ACAR but upregulated in late ACAR. Unsupervised clustering of these 18 genes distinguished mice with healthy grafts (syngeneic or allogeneic before adoptive T cell transfer), pre-symptomatic ACAR (allogeneic day 7), and late ACAR (allogeneic day 13) (FIG. 12E). Principal component loadings of these 18 genes in unsupervised clustering identified Gnb4, Sepw1, Gm16845, Rnf10, and Git1 as the top differentiators (FIGS. 20B, C).

The scaffolds are shown herein to provide a novel individualized measure of ACAR. As the scaffold can be remotely biopsied or explanted, longitudinal gene expression can be individualized by monitoring the change in expression over time for a given recipient. With this individualized gene expression change, ACAR-both pre-symptomatic and late symptomatic-can be distinguished from healthy syngeneic graft recipients with an Elastic Net-derived 6 gene panel (FIGS. 12F,G; Table 1). Changes in Tmem234, Sfxn5, Zfp963, and Rcsd1 were upregulated in ACAR compared to healthy recipients; whereas Vwf and Gm 19897 became more downregulated. Notably, this six-gene panel differentiates the progression from pre-symptomatic to late symptomatic ACAR as strongly as it differentiates these two stages from the time-matched syngeneic recipients. The scaffold can both identify ACAR and distinguish between stages of ACAR.

### Scaffolds Predict Early Risk of ACAR in Heart Transplant Recipients

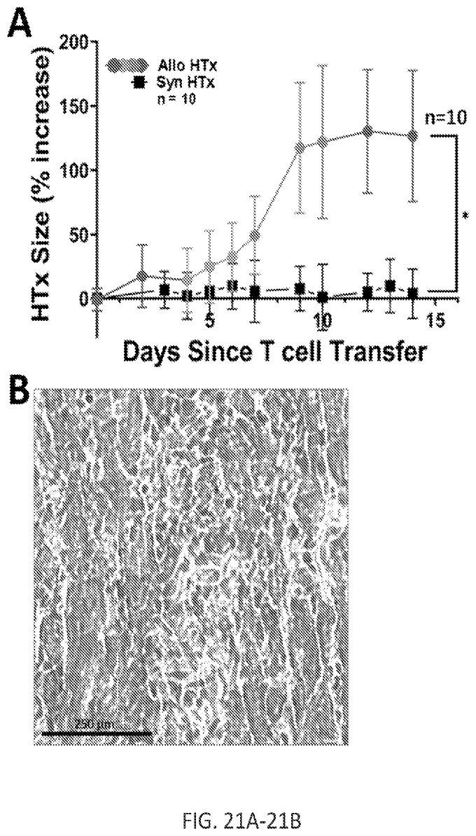

Predictive biomarkers of ACAR were also identified in murine heterotopic heart transplants (HTx) using cell-capture scaffolds (FIG. 13A). Allorecognition was initiated by adoptive transfer of 2×10⁷ syngeneic T cells from naïve B6 donors. Syngeneic heart graft recipients remained free of ACAR (FIGS. 13B,C). For allograft recipients, however, ACAR occurred over 14 days (FIGS. 13B,D; FIG. 21), with symptoms (i.e., edema and weakened pulse) beginning at day 8 or 9 and complete graft failure on day 13 or 14. Scaffolds were serially explanted from HTx recipients corresponding to ACAR state, with an earlier pre-symptomatic timepoint at day 5, a mid-rejection point on day 9, and a late, resolving rejection day 14 explant. 17 differentially expressed genes were identified as a sparse biomarker panel of HTx ACAR

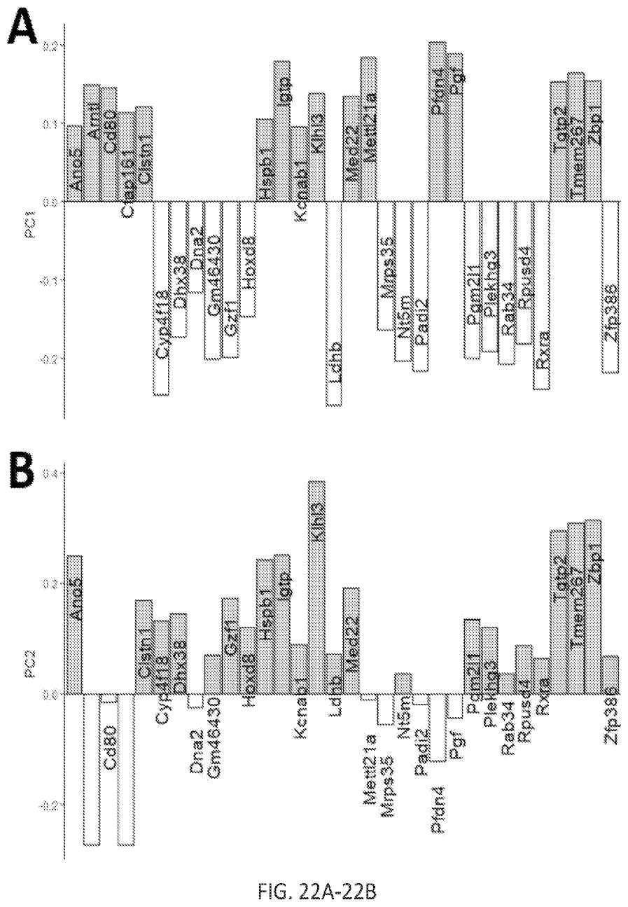

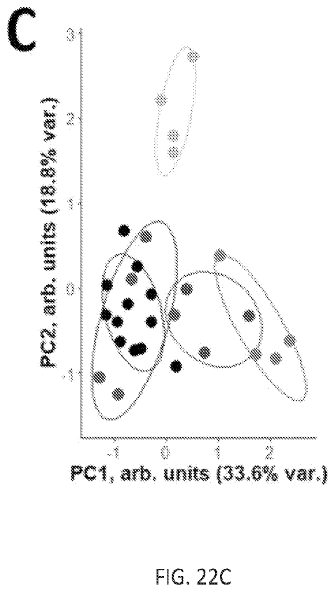

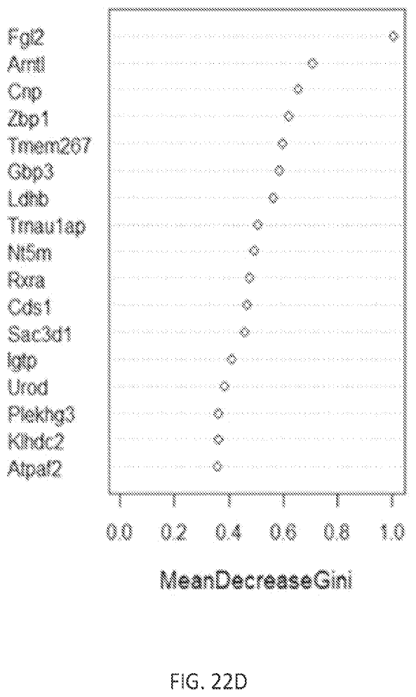

(FIG. 13E; FIGS. 22A-C; Table 2). Cds1, Ntrm, and Ldhb were moderately downregulated at the scaffold during all stages of ACAR. Atpaf2 and Arntl, by contrast, were strongly upregulated in pre-symptomatic ACAR compared to both healthy recipients and symptomatic ACAR, while Rxra, Urod, and Plekhg3 were downregulated in pre-symptomatic ACAR only. Lastly, the remaining 8 genes were strongly upregulated in mid-symptomatic ACAR only. This sparse gene panel distinguishes pre-symptomatic and mid-symptomatic ACAR from healthy recipients, and from one another.

TABLE 2
Pathways and genes differentially represented
at the scaffold during ACAR in HTx.

| HTx DEGs | HTx Panel | Canonical KTx Markers in HTx |
|---|---|---|
| Gstt1 | Tmem267 | Cd80 |
| Rab34 | Sac3d1 | Arg1 |
| Nr1h3 | Cnp | Hspa1a |
| Dbf4 | Klhdc2 | Pgf |
| Tbc1d8 | Igtp | Cxcl9 |
| Cyp4f18 | Atpaf2 | Gstt1 |
| Ruvbl2 | Arntl | Myo6 |
| Bid | Cds1 | Plvap |
| Pgf | Nt5m | Ptprd |
| Prg4 | Ldhb | Tcn2 |
| Zfp715 | Plekhg3 | Adora2b |
| Adora2b | Gbp3 | Cxcl5 |
| Qrsl1 | Zbp1 | Il1r2 |
| Arg1 | Trnau1ap | Tpbg |
| Ptprb | Rxra | Abcb1b |
| Jsrp1 | Urod | Ada |
| Tcn2 | Fgl2 | Commd9 |
| Pspc1 | | Dvl1 |
| Angpt1 | | Hoxd8 |
| Wdyhv1 | | Rpl32l |
| Arap3 | | |
| Sac3d1 | | |
| Rnaseh2c | | |
| Twnk | | |
| Gdf11 | | |
| Exosc7 | | |
| Mettl21a | | |
| Il1r2 | | |
| Traf1 | | |
| Egfl7 | | |
| Nmi | | |
| Slc43a3 | | |
| Hoxd8 | | |
| Map1a | | |
| Ndufaf1 | | |
| Gzf1 | | |
| E2f1 | | |
| Zbp1 | | |
| Ptprd | | |
| Trnau1ap | | |
| Padi2 | | |
| Abcb1b | | |
| Pank4 | | |
| Dvl1 | | |
| Trmt44 | | |
| Cds1 | | |
| Cxcl5 | | |
| Ldhb | | |
| Zfp626 | | |
| Gab1 | | |
| Snx19 | | |
| Rpusd4 | | |
| Nt5m | | |
| Mansc1 | | |
| Ypel4 | | |
| Uaca | | |
| Plvap | | |
| Dna2 | | |
| Trappc14 | | |
| Gpr160 | | |
| Ptger2 | | |
| Dhx38 | | |
| Tigar | | |
| Clstn1 | | |
| Mrps35 | | |
| Tex2 | | |
| Mapk1ip1 | | |
| Zbed3 | | |
| Zfp386 | | |
| Uggt2 | | |
| Mustn1 | | |
| Atpaf2 | | |
| Trp53rkb | | |
| AY074887 | | |
| Zfp553 | | |
| C87436 | | |
| Champ1 | | |
| Ankrd16 | | |
| Zbtb45 | | |
| Bag5 | | |
| Nlrc3 | | |
| Mical3 | | |
| Pfdn4 | | |
| Plekhg3 | | |
| Tnfaip6 | | |
| Exoc6 | | |
| Per2 | | |
| Hspa2 | | |
| Zfp954 | | |
| Ganc | | |
| Cdc42ep5 | | |
| Jchain | | |
| Sco1 | | |
| Tut1 | | |
| Plekhf1 | | |
| Cd80 | | |
| Igkc | | |
| Rpl23a.ps3 | | |
| Gm17251 | | |
| Igha | | |
| Rnf113a2 | | |
| Mir6236 | | |
| Gm43112 | | |
| Rpl321 | | |
| Gm46430 | | |
| Gm48887 | | |
| Gm7926 | | |

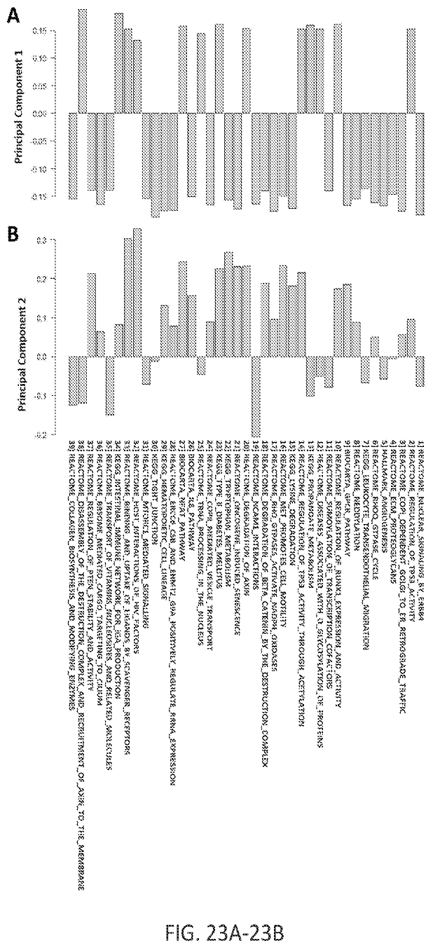

The biomarker panel was compared to the commercially available Allopmap™ test. The Allomap™ test is a blood-based assay of T cell proliferation, but lacks positive predictive power for allograft rejection (Parent et al., in The Pathology of Cardiac Transplantation: A clinical and pathological perspective, (Springer, 2017), pp. 265-277, Crespo-Leiro et al., CARGO II. Eur Heart J 37, 2591-2601 (2016), Kobashigawa, et al., Circulation: Heart Failure 8, 557-564 (2015). A robust, two-metric system was used to distinguish between healthy grafts or ACAR using singular value decomposition (SVD) for unsupervised scoring and a Random Forest bagged-tree approach for supervised scoring of samples (FIGS. 13F, G). The scaffold-derived panel distinguishes pre-symptomatic and mid-symptomatic ACAR from healthy graft recipients (FIG. 13F), while mouse orthologs of the Allomap™ test fail to distinguish scaffolds from mice with healthy HTx and ACAR (FIG. 13G). Fgl2, Arntl, and Cnp have the greatest variable importance in this two-metric system for identifying ACAR (FIG. 23D). Most notably, Fgl2 is downregulated in pre-symptomatic ACAR but upregulated in mid-symptomatic ACAR relative to both late ACAR and healthy graft recipients. Fgl2 plays a role in DC maturation, Treg-based regulation of T cell proliferation, and can be inhibited to ameliorate graft rejection. Early downregulation of Fgl2 may allow antigen presenting cell (APC) maturation, and its modulation to inhibit APC maturation could be valuable as a therapeutic target readily monitored by the remote scaffold. This early identification at the scaffold could create a therapeutic window for immune suppression interventions to abrogate ACAR before overt graft injury.

### Scaffolds Recapitulate Conserved Biomarkers of ACAR

Additionally, many genes identified through sequencing of clinical graft biopsies as canonical markers of graft rejection in kidney transplantation (Halloran et al., American Journal of Transplantation 18, 785-795 (2018), Halloran, et al,. American Journal of Transplantation 17, 1754-1769 (2017)) are also differentially expressed at the scaffold during HTx rejection (FIG. 13H). Cd80, Arg1, Hspala, Gstt1, Plvap, Ton2, and Il1r2 are expressed approximately uniformly during the stages of ACAR. The scaffold provides, however, a uniquely nuanced assessment of these canonical markers, as some, such as Adora2b, Ada, Commd9, and Hoxd8are most divergent in expression from the healthy recipients before ACAR symptoms present. Others still, such as Pgf, Cxcl9, Cxcl5, or Dvll differ from healthy expression most during mid-symptomatic rejection. Expression of these canonical markers was not significantly altered during late, resolving ACAR, when graft dysfunction is most readily observable. By sampling tissue-like immune responses at the scaffold, biomarkers of solid organ transplant rejection which are conserved across organ types—and their dynamics—can be identified.

### Scaffolds Identify Predictive Pathways and Biomarkers ACAR Independent of Tissue Type

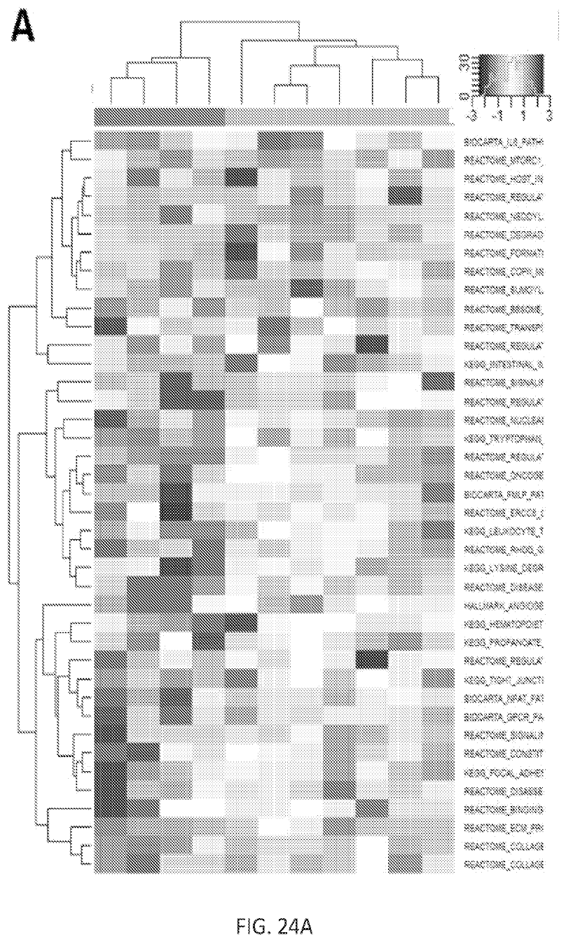

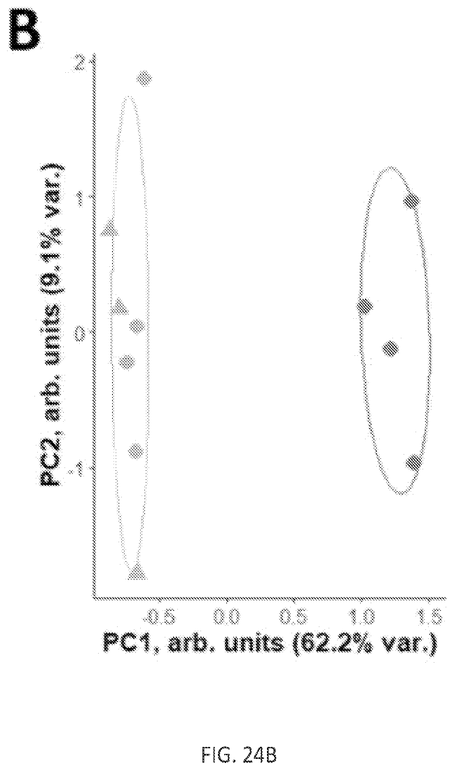

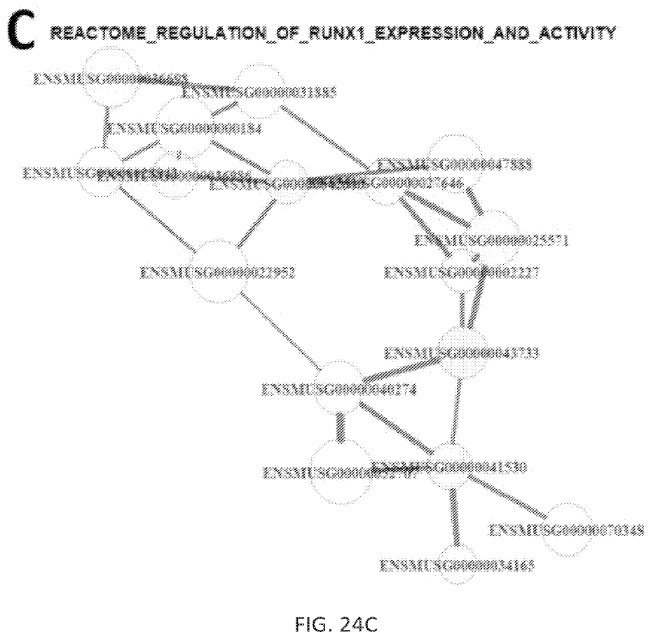

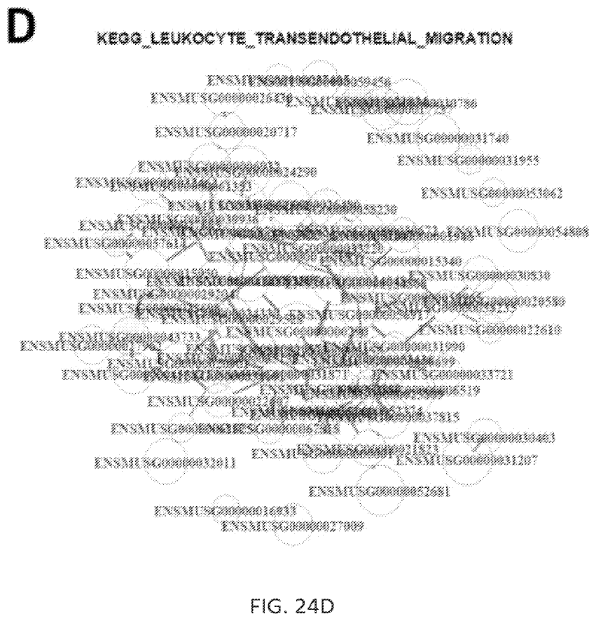

As many assays and biomarkers studied for use in surveilling allograft rejection generate false positives for other inflammatory insults, such as viral infection, a tissue-independent predictive panel of ACAR symptom onset was developed to engineer greater rejection specificity in the scaffold-derived biomarker panel. To do so, the two cohorts were batch normalized and gene expression was filtered. Gene expression was converted to gene set variation scores using the MSigDBR database, and an Elastic Net regression selected 25 pathways as a sparse, indicative panel (FIG. 14A). Interestingly, these 25 pathways at the scaffold distinguish both pre-symptomatic and mid-symptomatic ACAR from healthy HTx recipients, but with divergent clustering (FIG. 14B). This dissimilar pathway representation suggests a transition in ACAR state between HTx day 5 and day 9, potentially corresponding with the onset of symptoms. Among this sparse 25-pathway set, pathways associated with calcineurin and Vegf signaling are most differently represented by the two stages of ACAR (FIG. 23), with both being enriched in pre-symptomatic ACAR compared to healthy graft recipients or mid-symptomatic ACAR. 40 pathways with highly differential enrichment between only pre-symptomatic and mid-symptomatic ACAR were further identified (FIGS. 24A,B). The most dissimilarly enriched of these pathways are RunXI Expression and Activity and Leukocyte Transendothelial Migration (FIGS. 24C,D). Overall, these pathways differences between pre-symptomatic and symptomatic ACAR indicate that a different set of biological processes or processes being regulated very differently are acting as ACAR transitions to a symptomatic stage.

To develop a scaffold-based biomarker panel of ACAR that is independent of graft tissue and more non-specific to other inflammatory insults, genes with both a large fold change in expression between healthy recipients and ACAR and a low false discovery rate were identified in both STx and HTx (Table 3). 43 genes with high differential expression between ACAR recipients and healthy recipients in both transplant types were identified (FIG. 14C). Interestingly, this computational process results in a group of potential biomarkers that does not include the previously derived STx or HTx signatures, or any of the Allomap™ orthologs. Elastic Net regression reduced this group of strong differentiators to 13 genes (klf0) as a minimal panel for distinguishing ACAR in both STx and HTx recipients (FIG. 14E), in which late-stage ACAR was excluded from the regression of the gene panel to emphasize predicting symptom onset.

TABLE 3
Pathways and genes differentially represented at the
scaffold conserved during ACAR in both STx and HTx.

| | HTx and STx | HTx and STx |
|---|---|---|
| HTx and STx Differentially Enriched Pathways | DEGs | Panel |
| REACTOME_LAMININ_INTERACTIONS | Bcl6b | Klf9 |
| REACTOME_TRANSPORT_OF_VITAMINS_{—} | Mcm3ap | H2-Eb1 |
| NUCLEOSIDES_AND_RELATED_MOLECULES | | |
| REACTOME_DISEASES_ASSOCIATED_WITH_O_{—} | Col18a1 | Pura |
| GLYCOSYLATION_OF_PROTEINS | | |
| REACTOME_SIGNALING_BY_MET | Gria3 | Slc30a7 |
| KEGG_FOCAL_ADHESION | Rab34 | Prcp |
| REACTOME_HDR_THROUGH_SINGLE_STRAND_{—} | Napsa | Flywch1 |
| ANNEALING_SSA | | |
| REACTOME_HOMOLOGOUS_DNA_PAIRING_AND_{—} | Timmdc1 | Cbx7 |
| STRAND_EXCHANGE | | |
| REACTOME_PROCESSING_OF_INTRONLESS_PRE_{—} | Map2k7 | Prcc |
| MRNAS | | |
| REACTOME_BINDING_AND_UPTAKE_OF_{—} | Tbc1d8 | Nmi |
| LIGANDS_BY_SCAVENGER_RECEPTORS | | |
| REACTOME_SIGNALING_BY_NOTCH2 | Ralb | Arhgap9 |
| REACTOME_SIGNALING_BY_ERBB4 | Matk | Zfp182 |
| BIOCARTA_EIF4_PATHWAY | Ccl8 | Upf3a |
| BIOCARTA_NOS1_PATHWAY | Rnmt | Padi2 |
| BIOCARTA_CALCINEURIN_PATHWAY | Kdsr | |
| REACTOME_FOXO_MEDIATED_TRANSCRIPTION_{—} | Slc3a2 | |
| OF_CELL_CYCLE_GENES | | |
| HALLMARK_HYPOXIA | Zfp715 | |
| REACTOME_REGULATION_OF_RUNX1_{—} | Nelfb | |
| EXPRESSION_AND_ACTIVITY | | |
| REACTOME_REGULATION_OF_TP53_ACTIVITY_{—} | Cacfd1 | |
| THROUGH_ACETYLATION | | |
| REACTOME_DNA_DOUBLE_STRAND_BREAK_{—} | Bola1 | |
| RESPONSE | | |
| REACTOME_CELL_DEATH_SIGNALLING_VIA_{—} | Eef1a2 | |
| NRAGE_NRIF_AND_NADE | | |
| REACTOME_MITOTIC_PROPHASE | Med1 | |
| KEGG_VEGF_SIGNALING_PATHWAY | Pfn1 | |
| BIOCARTA_CARM_ER_PATHWAY | Mknk2 | |
| REACTOME_KSRP_KHSRP_BINDS_AND_{—} | Fkbp1b | |
| DESTABILIZES_MRNA | | |
| KEGG_SYSTEMIC_LUPUS_ERYTHEMATOSUS | Mink1 | |
| | Acot2 | |
| | Zfyve16 | |
| | Capn7 | |
| | Mrpl57 | |
| | Angpt1 | |
| | Parvb | |
| | Mroh1 | |
| | Vapa | |
| | Axin1 | |
| | Slc25a46 | |
| | Tapbp | |
| | Impact | |
| | Cd74 | |
| | Banf1 | |
| | Arl3 | |
| | Cbr2 | |
| | Mmp19 | |
| | Prim1 | |
| | Mettl21a | |
| | Slc39a10 | |
| | Pou2f1 | |
| | Abi2 | |
| | Fubp3 | |
| | Nmi | |
| | Hoxd8 | |
| | Gabpb1 | |
| | Bub1 | |
| | Bcl2l11 | |
| | E2f1 | |
| | Hps3 | |
| | Kcnab1 | |
| | Col15a1 | |
| | Tm2d1 | |
| | Hmgcl | |
| | Trnau1ap | |
| | Padi2 | |
| | Lrrc47 | |
| | Rchy1 | |
| | Naaa | |
| | Bhlhe41 | |
| | Ercc2 | |
| | Adm | |
| | Slc25a14 | |
| | Eif4ebp1 | |
| | Mt1 | |
| | Pdgfd | |
| | Trak1 | |
| | Kank1 | |
| | Klf9 | |
| | Fam151b | |
| | Polr3b | |
| | Uaca | |
| | Edil3 | |
| | Foxn2 | |
| | Neil2 | |
| | Lysmd3 | |
| | Colec12 | |
| | Fam13b | |
| | Ppfibp2 | |
| | Upf3b | |
| | H2-Aa | |
| | H2-DMa | |
| | Fermt2 | |
| | Vopp1 | |
| | Fbxw17 | |
| | Tgm2 | |
| | Cpe | |
| | Arl5c | |
| | Txnip | |
| | Upf3a | |
| | BC028528 | |
| | Isg20 | |
| | Znfx1 | |
| | Gsap | |
| | Flywch1 | |
| | Gbp7 | |
| | Zfp770 | |
| | Ankib1 | |
| | Ccnb1 | |
| | Mapk1ip1 | |
| | Ndufa7 | |
| | Tril | |
| | Pura | |
| | Rtn4rl1 | |
| | Camk2n1 | |
| | Zfp319 | |
| | Irgm1 | |
| | Mcmbp | |
| | Pigm | |
| | Cbx7 | |
| | Ifi205 | |
| | Arfgap3 | |
| | Rhob | |
| | Slc30a7 | |
| | Zfp182 | |
| | Cd47 | |
| | Mrps14 | |
| | C1qtnf3 | |
| | Psma3 | |
| | H2-Eb1 | |
| | Prcp | |
| | Ei24 | |
| | Phactr2 | |
| | Zfp780b | |
| | Zfp26 | |
| | Chchd1 | |
| | Hspbp1 | |
| | Cd59b | |
| | Treml2 | |
| | H2-Ab1 | |
| | Snrnp40 | |
| | Gas2l3 | |
| | Commd6 | |
| | Gm12359 | |
| | Smarcd2 | |
| | Zfp703 | |
| | Rasgef1b | |
| | Gm4890 | |
| | Gpr137b-ps | |
| | Rnf113a2 | |
| | Mir6398 | |

### Scaffolds Distinguish Pre-Symptomatic ACAR from Respiratory Infection

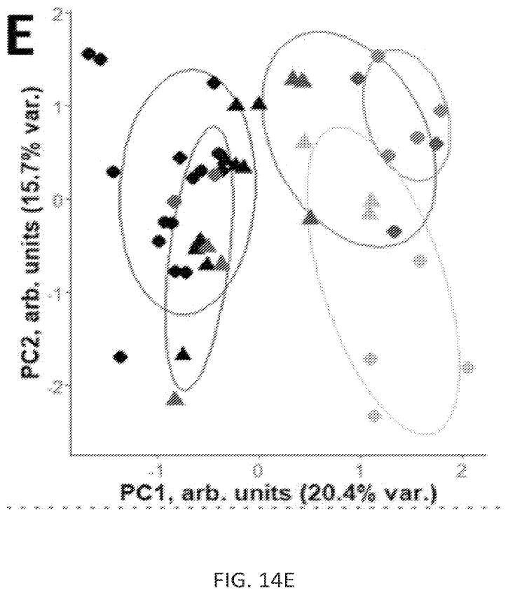

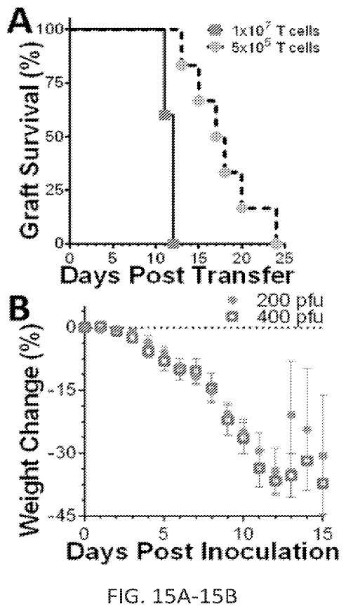

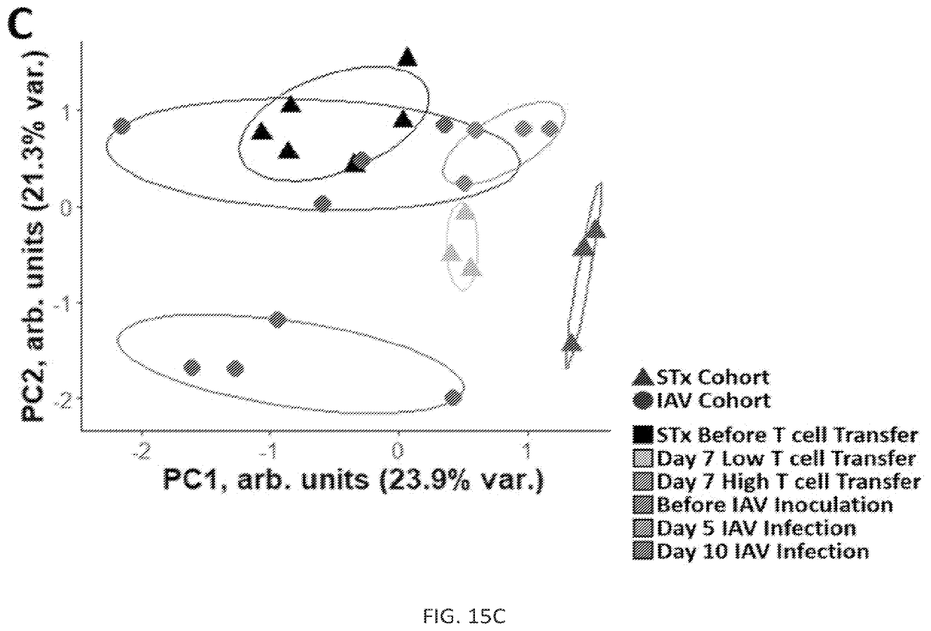

Allograft preservation occurs by suppressing the adaptive immune system, which can result in infections such as Influenza A Virus (IAV). Such viral infections limit the power of non-invasive assays at predicting ACAR and can even induce pro-rejection inflammation. The immunological pathways governing the early stages of ACAR may overlap with those associated with other inflammatory insults, and the tissue-independent gene panel developed in this study identified numerous genes of unknown immunological function. As such, the tissue-independent, scaffold panel of 13 genes developed herein was validated to identify presymptomatic STx ACAR in RAG2^{-/-} recipients following a high adoptive transfer of T cells (1×10⁷ cells) or a low transfer (5×10⁵ cells) (FIG. 15A). High T cell transfer STx recipients first exhibited graft wounding on day 10 or 11, as previously (FIGS. 11C,D), while low T cell transfer recipients had a more varied onset of wounding ranging from day 13 to day 24 after adoptive transfer. For assessing gene panel specificity towards ACAR, scaffolds were explanted from RAG2^{-/-} mice undergoing an H1N1 influenza infection in which high adoptive T cell transfer took place 7 days prior to viral inoculation (FIG. 15B). Weight loss was tracked following influenza inoculation as a relative measure of infection severity, with scaffolds explanted from the inoculated mice 3 days prior to inoculation and 5 days and 10 days post inoculation. 5 days post inoculation was associated with a moderate infection, while 10 days post inoculation was associated with a severe infection. Housekeeping genes were selected (FIG. 27). Expression of the trained model-derived 13 genes at these scaffold explants (FIG. 15C) identified high transfer STx recipients, 3-4 days prior to symptom onset, as a distinct group from scaffolds of those same STx recipients prior to T cell transfer. Day 7 scaffold explants from the low transfer, 9-13 days prior to symptom onset, were also group distinctly, though with weaker differentiation from the pre-transfer scaffolds. Scaffolds explanted both before and during moderate influenza infection were not distinguishable from the healthy STx scaffold explants. Conversely, scaffolds explanted during sever infection exhibited highly differential panel expression compared to all other groups. These gene expression results demonstrate the utility of a scaffold-derived biomarker panel in identifying ACAR prior to symptom onset, determining the tempo of ACAR onset, and distinguishing ACAR from a non-rejection inflammatory insult such as infection.

### Scaffolds Predict Onset of ACAR Symptoms in Immune Suppressed Wildtype Mice

The adoptive transfer model in RAG2^{-/-} mice employed to induce ACAR as a tunable event likely precludes or alters many endogenous T cell processes, such as central tolerance, proliferation, migration, and, possibly, activation. Similarly, by explanting scaffolds and performing the adoptive T cell transfer after the grafts have been given sufficient time to heal following transplant, and in the case of STx become neovascularized, the ischemia-reperfusion injury period immediately following transplantation was largely avoided, a period of great clinical need for surveilling for rejection. As such, studies were expanded to wildtype murine models of effective/inadequate immunosuppression in healthy recipients by explanting scaffolds from wildtype skin graft recipients which received T cell depleting monoclonal anti-Cd4 and anti-Cd8 antibodies. In these STx with varied doses of T cell depletion (FIG. 15D), rejection progressed over different kinetics to delay the onset of ACAR.

### DISCUSSION

Provided herein are immune cell-capturing scaffolds and methods of use thereof that predict symptomatic acute rejection in skin and heart allografts. These remotely implanted scaffolds capture predominantly innate immune cells (FIGS. 11G,H) and thus identify immune responses that precede graft destruction. Innate cell participation has been under-emphasized in contributing to allograft rejection, as immune suppressive drugs largely target T cell maturation or responses, which are highly localized. A potential advantage of the scaffold implant lies in capturing innate cells which are more systemic and precede T cell responses (FIG. 11H). These scaffolds likely intravasate predominantly innate immune cells as inflammation surrounding the implant is altered by systemic physiology and pathology. This results in an immunological niche with similar cell infiltrates to other inflammatory sites.

Enriched pathways at the scaffold during acute rejection, particularly pre-symptomatic rejection, involve these myeloid cells (FIG. 12B). Notably, these innate cell pathways are identified at the pathway level through gene expression, but differences between acute rejection and healthy graft recipients are not identified through cell surface markers alone or cell type ratios (FIG. 11H, FIG. 17) in circulation, at the scaffold, or at the graft itself. Resultingly, gene expression at the scaffold holds value as a therapeutic tool to identify graft rejection prior to injury. As the scaffold implant facilitates facile, remote biopsy, greater capacity for frequent longitudinal monitoring is possible than with graft biopsies. Accordingly, the scaffolds described herein provide for a unique therapeutic window when immune activation occurs prior to significant damage to the transplant organ, which enable precision immune suppression facilitating improved patient outcomes.

The scaffolds provided herein provide a tool to understand the biology of organ transplant rejection that facilitates identification of novel therapeutic strategies or targets. The scaffold analysis provided herein has yielded pathways and cellular transcripts of immune cells that are not typically impacted by current immune suppression, which could serve as targets for novel anti-rejection therapeutics at a window when personalized therapeutic intervention can preserve the graft prior to significant damage

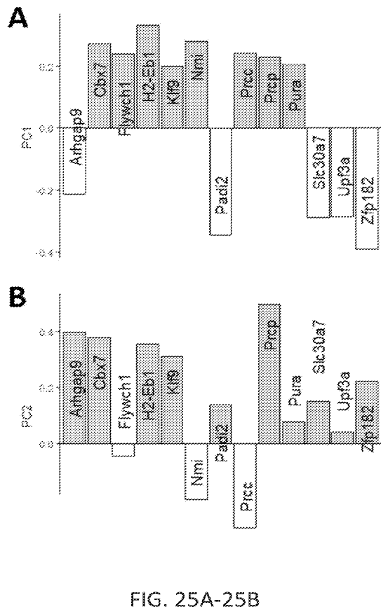

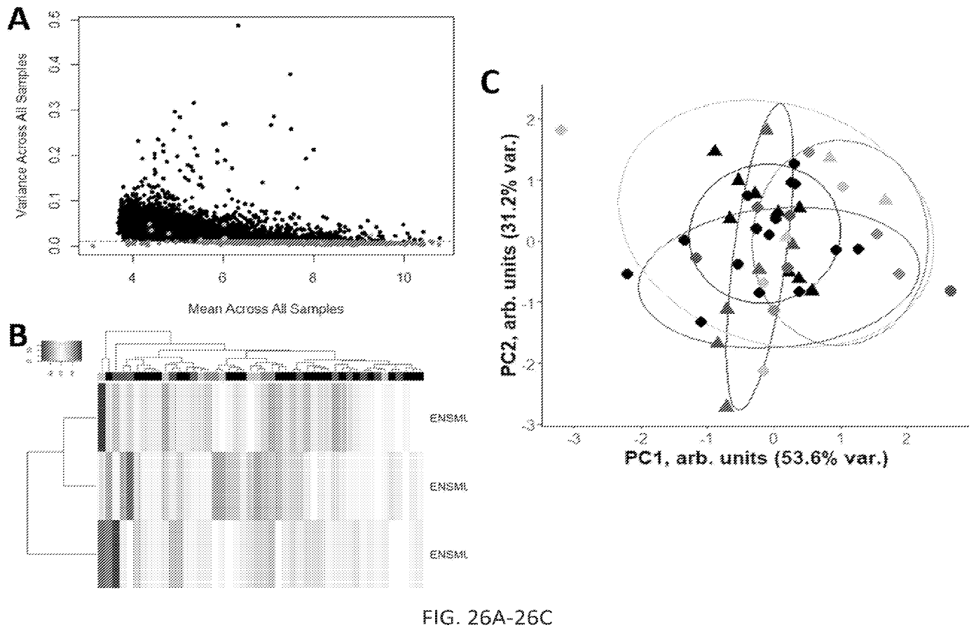

Pathway enrichment and gene expression in the scaffold recapitulate ACAR dynamics in tissue-specific and tissue-independent contexts that can predict symptom onset. These gene expression patterns have prognostic value. Pathways associated with rejection are enriched in the scaffold prior to symptomatic rejection (FIGS. 12B,C; FIG. 20, FIG. 25), and pathways are differentially enriched between pre-symptomatic rejection and mid-symptomatic rejection (FIGS. 13A,B; FIG. 25). Sparse gene panel signatures of acute rejection in skin grafts (FIGS. 12D,E) and heart grafts (FIG. 13E) were developed and conserved in both models of graft rejection for greater biological specificity (FIG. 14C-E). This 13-gene panel distinguished fast and slow rejection tempos each from healthy graft recipients in our adoptive transfer T cell model with specificity against inflammatory insult due to respiratory infection (FIG. 15C) and in wildtype mice undergoing immune suppression (FIG. 15E). Without wishing to be bound by theory, it is possible that this early identification of acute rejection is due to innate immune cell intravasation to the scaffold with tissue residency-like phenotype more specific to allogeneic rejection that can be identified in circulation. Solid organ transplant is performed at significant expense, and the improved graft surveillance tools described herein can substantially prolong function, reducing long-term expenses. There is a paucity of tools to predict rejection, and existing tools such as Allomap and Allosure are “rule-out” methods only. Moreover, the scaffold-derived gene expression signatures described herein outperform Allomap murine orthologs and is specific to transplant rejection compared to viral infection. As such, gene expression at the scaffold implant provides a positive predictive diagnostic tool to personalize therapeutic strategy.

### MATERIALS AND METHODS

Materials. Poly(ε-caprolactone), ester terminated (Evonik Corp, Birmingham, AL) was purchased for generating microporous scaffolds. Suture (Ethicon), carprofen, and punch biopsies (Henry Schein Inc.) were purchased to perform scaffold implants, skin or heart transplants, and scaffold biopsy in mice. An H&E staining kit (Abcam, Cambridge, UK) was purchased to stain histological samples, and a direct-zol RNA Miniprep Plus (Zymo Research) was purchased to isolate RNA from tissue and scaffold samples. Naive pan mouse T cell isolation kit and LS columns were purchased from Miltenyi Biotec (Bergisch Gladbach, North Rhine-Westphalia, Germany). TaqMan Gene Probes, Gene Expression Mastermix, and all other reagents were purchased from Thermo Fisher Scientific (Waltham, MA) unless otherwise stated. To maintain integrity, materials were maintained at room temperature, 4° C., or −20° C. according to manufacturer instructions. Water from a Millipore filtration system (Darmstadt, Germany) with 18.2 MQΩ cm resistivity was used unless otherwise stated.

Microporous Scaffold Generation. Microporous polycaprolactone scaffolds were generated as described previously (Oakes et al., Cancer Res 80, 602-612 (2020), Rao et al., Cancer Res 76, 5209-5218 (2016). Bushnell et al., Cancer research 79, 2042-2053 (2019)). Briefly, microporous poly-caprolactone (PCL) scaffolds were generated by first mixing 99 gm of sieved NaCl (250-425 μm) with 3 gm of PCL pellets in a Ross Mixer at 85° C. for one hour. 77.5 mg of the PCL-NaCl melt dispersion was measured and pressed into 5 or 6-mm diameter scaffolds. Scaffolds were heated at 65° C. for 5 min on each side to anneal the PCL. Upon cooling, the scaffolds were immersed in water three times for one hour to dissolve the NaCl porogen. The scaffolds were then sanitized by ethanol immersion and washed with sterile water in preparation for murine implantation.

Heterotopic Heart and Skin Transplant Models. All animal care and procedures were performed in accordance with standards from the Guide for the Care and Use of Laboratory Animals and were carried out in compliance with protocols approved by the Institutional Animal Care and Use Committee (IACUC Protocol 00009777) at the University of Michigan (UM). Male and female C57BL/6, Balb/C, and B6 Cg-Rag2^{tml.lCgn} (RAG2^{-/-}) mice were purchased from Jackson Laboratory (Bar Harbor, ME) at 8-12 weeks old. Mice were housed in a pathogen-free environment under a 12 hour light-dark cycle. RAG2^{-/-} mice were bred, and offspring used as transplant recipients at 8-12 weeks of age. Six animal cohorts were performed as follows: heterotopic heart transplant training cohort, heterotopic heart transplant validation cohort, skin transplant training cohort, skin transplant validation cohort, skin transplant variable response cohort, and skin transplant wildtype immunosuppression cohort.

In murine skin transplants (STx), donor adult mice were euthanized as approved, and the tails were cleansed with 70% ethanol. The tails were transected at the base and a longitudinal incision to deglove the tail skin was made. This tail skin was then trimmed into circles of 8 mm diameter with a punch biopsy tool. Recipient adult mice were anesthetized as approved and received subcutaneous carprofen (5 mg/kg) before surgery. The dorsal skin of the recipient animal was depilated with ointment, rinsed with water, and then scrubbed with ethanol and betadine. An 6-mm diameter skin portion was excised with a punch biopsy tool. The donor graft was placed on the recipient site and attached to the graft bed by 8-0 suture. The graft area was then impregnated with triple antibiotic ointment and wrapped with gauze. After 24 hours, another dose of carprofen was given. Dressings were removed on postoperative day 5-7 and sutures removed on day 10. Skin transplant recipients were monitored for graft loss and euthanized at the time of graft rejection for analysis of pathological changes within the grafts. STx was performed as follows: wild-type C57BL/6 graft to RAG2^{-/-} recipient (allogeneic), wild-type Balb/C graft to RAG2^{-/-} recipient (syngeneic), wild-type C57BL/6 graft to wild-type C57BL/6 recipient (allogeneic), or wild-type Balb/C graft to wild-type C57BL/6 recipient (syngeneic). syngeneic skin grafts served as controls for nonspecific inflammation related to surgery. grafts were considered to be rejected at the time of sloughing or upon complete conversion to a hard avascular eschar.

Murine heterotopic heart transplants (HTx) were performed as described previously (61). Briefly, grafts were collected from adult donor mice. The heterotopic transplant was maintained in the recipient neck for monitoring the viability of the transplanted heart by inspection and palpation. HTx was performed as wild-type C57BL/6 graft to RAG2^{-/-} recipient (allogeneic) or wild-type Balb/C graft to RAG2^{-/-} recipient (syngeneic). The donor heart ascending aorta and pulmonary artery were anastomosed to the recipient common carotid artery and external jugular vein, respectively. A prophylactic dose of antibiotics was administered to reduce the risk of infection. Analgesic carprofen was administered preemptively and every day for 48 hours, then as needed. During the initial 72 hours post-op, animals were monitored to confirm activity and health. Graft failure was determined by the lack of a palpable heartbeat. This HTx model is the standard for studying allograft responses.

Scaffold Implants in Transplant Recipients. Mice were anesthetized with isoflurane for subcutaneous implantation of scaffolds and serial scaffold explants. Mice received carprofen immediately before surgery and 24 hours after surgery. Four to six scaffolds were implanted subcutaneously 14 days prior to inducing graft rejection in RAG2^{-/-} mice or prior to transplant in immunocompetent mice. After vascularization and cell in-growth, scaffolds were then explanted before and during graft rejection. Blood (serial points) and primary grafts (terminal points) were also collected.

Inducing Graft Rejection. Transplant recipient RAG2^{-/-} mice were allowed 10 or 14 days for heart or skin grafts to heal, respectively, with another 14 days for implanted scaffolds to become vascularized and additional graft healing. For adoptive transfer induction of acute cellular allograft rejection (ACAR), C57BL/6 donor mice were euthanized, and splenic pan naive T cells were collected via magnetic-activated cell sorting. These syngeneic C57BL/6 T cells were counted with an automated cell counter (Countess 3 FL, Invitrogen, Waltham, MA) and injected i.p. into transplant recipient RAG2-/-mice. For skin transplants, 500,000 or 10 million cells were injected per mouse; while for heart transplants, 20 million cells were injected per mouse. For these adoptive transfer studies, the day of T cell transfer was considered day 0, and graft recipients were monitored for graft rejection and euthanized at the time of rejection to analyze the pathological changes at the cellular and molecular level. Heart graft rejection was scored on a 0-3 scale: 0=strong pulse, no swelling; 1=weak pulse or swelling, 2=weak pulse and swelling, 3=no pulse. Skin graft rejection was scored as percentage of the area of the graft that was visibly inflamed.

Histology. For histological analyses, skin, heart, and scaffold samples were each embedded into OCT-30% sucrose and sectioned onto slides using a Cryostat (Microm HM 525, Thermo Fisher Scientific, Waltham, MA) at 14 μm thickness. The slides were stained with hematoxylin and eosin following standard procedures. Images were taken at 20x within the thickness of the scaffold. A board-certified pathologist, initially blinded to sample source, read and analyzed the slides for evidence of transplant rejection and inflammation; the pathologist scored these samples and then subsequently participated in discussions on interpreting the results from the different samples.

Tissue and Nucleic Acid Isolation. To collect serial biopsies of PCL scaffold implants, implanted mice were anesthetized with isoflurane and shaved closely near the implant site. A small dorsal incision made aside the surface of the implanted scaffold was opened with sterile forceps, with the implanted scaffold and encapsulating tissue pulled through and excised. The incision was then sutured closed. Implants for RNA isolation or histology were flash frozen in isopentane, kept on dry ice, and stored in a labeled tube at −80° C. until further processing and analysis. Implants used for flow cytometry were immediately placed into PBS kept on ice. For serial blood collection, mice were restrained, and a 4-mm mouse phlebotomy lancet was used to pierce the submandibular region of the mouse. Blood was allowed to drip into the collection vial.

Gauze compression was applied after collection, when needed, to stop blood loss. To collect heart or skin graft biopsy and larger blood quantities, mice were anesthetized and blood terminally drawn via intracardiac draw with EDTA to prevent clotting. The mice were then euthanized as approved, and skin or heart grafts were carefully excised from surrounding tissue. All blood samples were resuspended in ACK lysing buffer to lyse red blood cells (RBCs), washed in PBS, and then resuspended in Trizol Reagent and stored at −80° C. until further processing and analysis. Frozen tissues were homogenized in TRIzol Reagent, and RNA was isolated with a direct-zol RNA Miniprep Plus kit following manufacturer instructions.

Flow Cytometry. Biopsied scaffolds were prepared for flow cytometry by mechanical dissection and enzymatic incubation. Briefly, samples were minced, incubated for 20 min in Liberase TL (Roche) at 37° C., then mashed through a 70 μm filter which was washed extensively with FACS buffer: PBS (Life Technologies) with 0.5% bovine serum albumin (Sigma Aldrich) and 2 mM EDTA (Gibco). Cells were equally split into two tubes to enable staining and analysis of innate and adaptive immune cells from the same scaffold and then blocked with anti-CD16/32 (1:50, clone 93, eBioscience). Each tube was stained with Live/Dead Fixable Red (Life Technologies) and Alexa Fluor® 700 anti-CD45 (1:125, clone 30-F11, Biolegend). The adaptive immune panel was also stained with: FITC anti-CD8 (1:25, clone 53-6.7, Biolegend), Pacific BlueTM anti-CD19 (1:100, clone 6D5, Biolegend), PE-Cy7 anti-CD49b (1:30, clone DX5, Biolegend), and V500 anti-CD4 (1:100, clone RM4-5, BD Biosciences). The innate immune panel was also stained with: APC anti-CD11c (1:80, clone N418, Biolegend), FITC anti-Ly6C (1:100, clone HK.14, Biolegend), Pacific Blue™ anti-Ly-6G/Ly-6C (Gr-1) (1:70, clone RB6-8C5, Biolegend), PE-Cy7 anti-F4/80 (1:80, clone BM8, Biolegend), and V500 anti-CD11b (1:100, clone M1/70, BD Biosciences). Samples were analyzed on a Cytoflex Cell Analyzer, and all single-color controls and FMOs were used to aid with gating and compensation (SI), with subsequent data analysis on FlowJo. Skin grafts, blood, and spleens from transplant recipient mice were similarly analyzed. Blood was collected via cardiac puncture, and a single cell splenocyte homogenate was obtained by mashing through a 70 μm filter which was washed extensively with FACS buffer. Red blood cells were then lysed with ACK lysis buffer (Gibco). The panel above was used for analysis, except no anti-CD49b antibody was used.

RNA Sequencing. For high-throughput gene expression analysis, bulk RNA sequencing (RNAseq) analysis and quality control were performed on a fee-for-service basis by the UM Advanced Genomics Core. The core uses a NextSeq HO 150 cycle and the QuantStudio 12k Flex system (ThermoFisher Scientific). For RNAseq a total of n=6 samples per condition (rejecting or healthy) were analyzed from each time point (day 0, 5, 9, or 14). Each sample was from an independent mouse and samples alternated between healthy and rejecting in their placement on the plate to minimize spatial bias. Samples were analyzed first to remove genes that did not have expression in more than half of the samples in each condition. Mple

Comparing Genes with Public Datasets and Pathway Analysis. Genes identified as significant via t-test were converted to their human orthologs using biomaRt (63). Pre-ranked gene lists were assembled for significant genes (adjusted p-value<0.05) using the LFC between healthy and rejecting transplant recipients as the ranking variable. The corresponding human ortholog genes underwent gene set enrichment analysis (GSEA) using a pre-ranked gene list to identify significantly enriched pathways (Subramanian et al., PNAS 102, 15545-15550 (2005)). Gene sets were sampled from the hallmark, BIOCARTA, KEGG, REACTOME, PID, and gene ontology (GO) databases obtained from the Molecular Signatures Database (MSigDB) collections. GSEA was performed using the GSEA v4.3.2 software (Broad Institute), computing gene-set overlaps between canonical pathways (CP) and GO biological process (BP). GSVA (Hänzelmann, R. Castelo, J. Guinney, GSVA: gene set variation analysis for microarray and RNA-Seq data. BMC Bioinformatics 14, 1-15 (2013)).

Differential Expression and Selecting Genes of Interest. Genes of interest were selected by log2 fold change (LFC)>1.5, and p_{adj}<0.1, attempting to include both genes that increased and decreased in expression during rejection to make the model more robust. Gene expression was then analyzed by elastic net regularization for high-dimensional differential expression across mice, allogeneic or syngeneic graft donor, and time points in the HTx and STx training cohorts to generate a biomarker panel, approximately 10-24 genes which categorize mice by graft health. Through singular value decomposition and supervised machine learning (Random Forest™) (L. Breiman, Random Forests. Machine Learning 45, 5-32 (2001)), to derive single metric scores and construct a predictive model based on biomarker panel expression for the probability that a mouse will reject the transplanted graft. Three housekeeping genes were also selected by analyzing each of the 16 OpenArray reference genes for lowest variance overall and across transplant type to select the three most stable reference genes (Gapdh, Ppia, Trib3) to be used as reference in RT-qPCR.

Panel Validation via RT-qPCR. Once these genes of interest were identified as described above, validation and prediction studies were conducted via RT-qPCR analysis in 384-well plates. Single-strand complementary DNA was synthesized from isolated RNA with the SuperScript™ VILO™ cDNA Synthesis Kit according to manufacturer instructions using a thermocycler (Mastercycler gradient, Eppendorf, Hamburg, Germany). RT was performed with RNA concentrations of 200 ng/uL. Because RNA isolates from blood typically did not achieve this concentration, an RNA clean-up kit was used to increase concentration and purity according to manufacturer instructions (RNA Clean & Concentrator-5, Zymo Research). TaqMan probes of the genes of interest were ordered, and cDNA samples placed in randomly assigned order on the 384-well plate. TaqMan Gene Expression Mastermix was used and a final volume of 9 μL was reached for each well of the 384-well plate. RT-qPCR was performed on the QuantStudio ViiA 7 system (Applied Biosystems) and Cq values determined by the accompanying software. Non-detects were left blank for any statistical analyses, but were filled with the median of all samples for SVD (because it requires complete matrices). Samples from the original RNAseq data were run on every 384-well plate to allow for a correction factor to be applied to account for variability. ΔCq values were calculated for each gene from the average of the reference genes for that sample.

Influenza Inoculation. Mice were lightly anesthetized with isoflurane. Mice were given 75, 200, or 400 plaque forming units of influenza A virus in 40 μL of PBS intranasally. Mice were monitored daily following infection and were euthanized after loss of 30% of their pre-infection body weight.

Wildtype T cell Depletion Study. mAbs were purchased from Bio X Cell. Anti-CD4 and anti-CD8 were given in combination on days-3 and-1 before skin graft at 200 μg per mAb per injection.

Statistical Analyses and Reproducibility. Statistical analyses for validation assays were performed using R and GraphPad Prism 9 software (GraphPad) when n>2 with unpaired Student's t-test or one- or two-way -with Tukey's multiple comparisons test. P values lower than 0.05 were considered statistically significant. For two-group comparisons, student's t tests were used to analyze flow cytometry data as comparisons were made between healthy and rejecting within each time point (no comparisons over time). ROC curves were plotted and analyzed in GraphPad. Box plots show the median, 25-75th percentiles and most extreme data points not considered outliers (outliers are indicated by red+). All data is presented as means±s.e.m. with the number of samples provided (distinct samples or mice (n) or images within a tissue (n); not repeated measures), unless otherwise indicated. The details of specific statistical tests used for each experiment and probability values are detailed in the figure legends and raw data and details of statistical analyses are provided in the Source Data. For histological images, at least three scaffold or graft samples were acquired and representative images displayed. For multiple group analysis and when comparing pooled control recipients vs rejecting recipient mice, two-way ANOVA was used with a post-hoc Bonferroni correction to determine significance. Log-rank test (Mantel-Cox) test was used for survival curve comparison. P values below 0.05 were considered statistically significant.

1. A method comprising:
a) obtaining at least one sample from a microenvironment of a synthetic scaffold implanted in a subject, wherein the subject has received a transplant; and
b) measuring an expression level or amount of at least one RNA, at least one gene, at least one cell type, and/or at least one protein in the at least one sample.

2. (canceled)

3. (canceled)

4. The method of claim 1, wherein the at least one sample is obtained no more than 7 days after the subject has received the transplant.

5. The method of claim 1, wherein the at least one sample is obtained about 14 days after the subject has received the transplant, about 21 days after the subject has received the plant, about 4 weeks after the subject has received the transplant, about 5 weeks after the subject has received the transplant, about 6 weeks after the subject has received the transplant, about 7 weeks after the subject has received the transplant, about 8 weeks after the subject has received the transplant, about 9 weeks after the subject has received the transplant, about 10 weeks after the subject has received the transplant, about 11 weeks after the subject has received the transplant, about 12 weeks after the subject has received the transplant, about 13 week after the subject has received the transplant, about 14 weeks after the subject has received the transplant, about 15 weeks after the subject has received the transplant, or about 16 weeks or more since the subject has received the transplant.

6. The method of any claim 1, wherein the at least one sample comprises a first sample obtained at a first time point after the subject has received the transplant and a second sample obtained at a second time point after the subject has received the transplant.

7. The method of claim 6, wherein the first sample is obtained within 7 days after the subject has received the transplant, and wherein the second sample is obtained at least 24 hours after the first sample.

8. (canceled)

9. The method of claim 1, wherein the second sample is obtained after the first sample.

10. (canceled)

11. (canceled)

12. (canceled)

13. The method of claim 1, comprising measuring an expression level of a panel of genes in the at least one sample, wherein the panel of genes comprises at least 3 genes.

14. The method of claim 13, wherein the panel of genes comprises 3-50 genes or wherein the panel of genes comprises 6-25 genes.

15. (canceled)

16. A method of treating transplant rejection in a subject, the method comprising:
a) obtaining at least one sample from a microenvironment of a synthetic scaffold implanted in a subject, wherein the subject has received a transplant;
b) measuring an expression level or amount of at least one RNA, at least one gene, at least one cell type, and/or at least one protein in the at least one sample to determine transplant rejection status in the subject; and
c) providing an anti-rejection therapy to the subject determined to be at risk of or currently experiencing transplant rejection, wherein:
i) an increase or a decrease in the expression level or amount of the at least one RNA, at least one gene, at least one cell type, and/or at least one protein compared to a reference level indicates that the subject is at risk of or currently experiencing transplant rejection, or
(ii) the at least one sample comprises a first sample obtained at a first time point after the subject has received the transplant and a second sample obtained at a second time point after the subject has received the transplant, and an increase or a decrease in the expression level or amount of the at least one RNA, at least one gene, at least one cell type, and/or at least one protein in the second sample compared to the first sample indicates that the subject is at risk of or currently experiencing transplant rejection.

17. (canceled)

18. (canceled)

19. The method of claim 16, wherein the at least one sample is obtained no more than 7 days after the subject has received the transplant.

20. The method of any claim 16, wherein the at least one sample is obtained about 14 days after the subject has received the transplant, about 21 days after the subject has received the transplant, about 4 weeks after the subject has received the transplant, about 5 weeks after the subject has received the transplant, about 6 weeks after the subject has received the transplant, about 7 weeks after the subject has received the transplant about 8 weeks after the subject has received the transplant, about 9 weeks after the subject has received the transplant, about 10 weeks after the subject has received the transplant, about 11 weeks after the subject has received the transplant, about 12 weeks after the subject has received the transplant, about 13 weeks after the subject has received the transplant, about 14 weeks after the subject has received the transplant, about 15 weeks after the subject has received the transplant, or about 16 weeks or more since the subject has received the transplant.

21. (canceled)

22. (canceled)

23. (canceled)

24. The method of claim 16, wherein the first sample is obtained within 7 days after the subject has received the transplant, and wherein the second sample is obtained at least 24 hours after the first sample.

25. The method of claim 16, wherein the second sample is obtained at least 2 weeks, at least 4 weeks, at least 6 weeks, at least 8weeks, at least 12 weeks, or at least 16 weeks after the first sample.

26-31. (canceled)

32. The method of claim 16, comprising measuring an expression level of a panel of genes in the at least one sample, wherein the panel of genes comprises at least 3 genes.

33. The method of claim 32, wherein the panel of genes comprises 3-50 genes or wherein the panel genes comprises 6-25 genes.

34. (canceled)

35. The method of claim 1, wherein the transplant is an allogeneic transplant or a xenogeneic transplant.

36. (canceled)

37. The method of claim 1, wherein the transplant is a heart transplant or a skin transplant.

38. (canceled)

39. The method of claim 1, wherein the subject has received at least one anti-rejection therapy.

40. The method of claim 39, wherein the at least one anti-rejection therapy is selected from an immunosuppressive agent and/or an antibody.
